(19) United States
(12) Patent Application Publication
(10) Pub. No.: US 2012/0270807 A1
(43) Pub. Date: Oct. 25, 2012

(54) MACROCYCLIC INHIBITORS OF SERINE PROTEASE ENZYMES

(76) Inventors: Éric Marsault, Sherbrooke (CA); Olivier Leogane, Brossard (CA); Axel Mathieu, Sherbrooke (CA); Sylvie Beaubien, Sherbrooke (CA)

(21) Appl. No.: 13/503,426

(22) PCT Filed: Oct. 22, 2010
(86) PCT No.: PCT/US10/53754

§ 371 Date: Jul. 9, 2012

Related U.S. Application Data
(60) Provisional application No. 61/254,434, filed on Oct. 23, 2009.

(51) Int. Cl. A61K 38/12; C07K 5/02

(52) U.S. Cl. 514/21.1; 530/317

(57) ABSTRACT

The present invention relates to novel macrocyclic compounds and salts thereof that bind to and/or are inhibitors of serine protease enzymes. The present invention also relates to intermediates of these compounds, pharmaceutical compositions containing these compounds and methods of using the compounds. These compounds are useful as therapeutics for treatment and prevention of a range of disease indications including hyperproliferative disorders, in particular those characterized by tumor metastasis, inflammatory disorders, skin and tissue disorders, cardiovascular disorders, respiratory disorders and viral infections.

### CROSS REFERENCE TO RELATED APPLICATION

This application claims priority to U.S. Provisional Application Ser. No. 61/254,434, filed Oct. 23, 2009, the disclosure of which is incorporated herein by reference in its entirety.

### FIELD OF THE INVENTION

The present invention relates to novel macrocyclic compounds and pharmaceutically acceptable salts thereof that bind to and/or are modulators, in particular inhibitors, of serine protease enzymes. The present invention also relates to intermediates of these compounds, pharmaceutical compositions containing these compounds and methods of using the compounds. The compounds are useful as therapeutics for treatment and prevention of a range of disease indications including hyperproliferative disorders, in particular those characterized by tumor metastasis, inflammatory disorders, skin and tissue disorders, cardiovascular disorders, respiratory disorders and viral infections.

### BACKGROUND OF THE INVENTION

Serine protease enzymes are involved in a number of key physiological processes in mammals, viruses, bacteria and other organisms, regulating such diverse functions as tissue homeostasis and repair, development, immunity and fertility, as well as others. On a biochemical level, these proteases are responsible for activation of hormones, growth factors, cytokines and other endogenous physiological messengers, regulation of ion channels, activation of receptors and control of cellular permeability.

Due to this array of actions, serine proteases have become targets for the development of pharmaceuticals. (Drews, J.; Ryser, S. Nat. Biotech. 1997, 15, 1318-1319; Imming, P.; Sinning, C.; Meyer, A. Nat. Rev. Drug Disc. 2006, 5, 821-834.) Indeed, it has been estimated that 3-4% of all druggable biological targets are members of this class. (Hopkins, A. L.; Groom, C. R. Nat. Rev. Drug Disc. 2002, 1, 727-730.) Specifically, inhibitors of these enzymes have proven to possess a wide range of pharmaceutically relevant activities as effective cardiovascular modulators, respiratory disease treatments, anti-inflammatories, antiviral agents and CNS drugs. Additionally, the intimate involvement of serine proteases in the maintenance processes for various tissues makes them emerging targets for cancer (Bialas, A.; Kafarski, P. Anti-cancer Agents Med. Chem. 2009, 9, 728-762), as well as skin diseases and disorders (Meyer-Hoffert, U. Arch. Immunol. Ther. Exp. 2009, 57, 345-354).

Among the more insidious characteristics of cancer cells is their ability to spread, or metastasize, to other sites in the body. In many cases, the ability of a tumor to metastasize is correlated with prognosis as tumors with high metastatic character lead to poor outcomes. Increased levels of proteolytic activity have been associated with cancer progression and metastasis. Serine proteases, among other proteolytic enzymes, contribute to degrading cellular structures and to tissue remodeling, thereby assisting with cancer invasion and spread. Further, proteases are involved in the activation of a host of growth factors that are required for stimulating the proliferation of cancer cells or angiogenesis. Some of the serine proteases involved in this process are urokinase, plasmin, elastase, thrombin and cathepsin G. Distinct substrate specificities have been found for proteases involved in cancer, suggesting that selected targeting of these proteases would be possible. (Beliveau, F.; Desilets, A.; Leduc, R. FEBS J. 2009, 276, 2213-2226.) In addition, an emerging class of serine proteases called the type II transmembrane serine proteases (TTSPs) has been found to be important in tissue homeostasis and in cancer, in particular with tumor metastasis. (Wu, Q. Curr. Top. Develop. Biol. 2003, 54, 167-206; Qui, D.; Owen, K.; Gray, K.; Bass, R.; Ellis, V. Biochem. Soc. Trans. 2007, 35, 583-587.) Members of the TTSP family also have roles in physiological processes as diverse as digestion, cardiac function, blood pressure regulation and hearing. (Bugge, T. H.; Antalis, T. M.; Wu, Q. J. Biol. Chem. 2009, 284, 23177-23181.) In these roles, TTSPs typically serve to maintain homeostasis and are often involved in hormone or growth factor activation or in the initiation of proteolytic cascades. In addition, more recent findings suggest that influenza and other respiratory viruses, such as human metapneumovirus, exploit TTSPs to promote their spread, making these proteases potential targets for intervention in viral infections. (Choi, S.-Y.; Bertram, S.; Glowacka, I.; Park, Y. W.; Pohlmann, S. Trends Mol. Med. 2009, 15, 303-312.)

TTSPs are characterized by short N-terminal tails that remain in the cytoplasm, a membrane-spanning region, the ligand binding domains and a serine protease domain at the C-terminus. Such features make them ideal for interaction with other cell surface proteins and components of adjacent cells.

One member of this enzyme class, matriptase (matriptase-1, MT-SP1, TADG-15, epithin, ST14), is a trypsin-like serine protease expressed by cells of epithelial origin and overexpressed in a wide variety of human cancers. (U.S. Pat. No. 5,482,848; U.S. Pat. No. 5,792,616; U.S. Pat. No. 5,972,616; U.S. Pat. No. 6,649,741; U.S. Pat. No. 7,030,231; U.S. Pat. No. 7,227,009; U.S. Pat. No. 7,276,364; U.S. Pat. No. 7,291,462; WO 99/42120; WO 00/53232; WO 01/23524; WO 01/29056; WO 01/57194; WO 01/36604; US 2003/0119168; US 2006/0099625; US 2008/0051559; Takeuchi, T.; Shuman, M. A.; Craik, C. S. Proc. Natl. Acad. Sci. 1999, 96, 11054-11061; Lin, C. Y.; Anders, J.; Johnson, M.; Sang, Q. A.; Dickson, R. B.; J. Biol. Chem. 2001, 274, 18231-18236; Oberst, M.; Johnson, M.; Dickson, R. B.; Lin, C.-Y. Recent Res. Develop. Biochem. 2002, 3, 169-190; Lin, C.-Y.; Oberst, M.; Johnson, M.; Dickson, R. B. Handbook of Proteolytic Enzymes, 2^{nd} ed., Barrett, A. J.; Rawlings, N. D.; Woessner, J. F., Elsevier: London, 2004, pp 1559-1561; List, K.; Bugge, T. H.; Szabo, R. Mol. Med. 2006, 12, 1-7; Lee, M.-S.; Johnson, M. D.; Lin, C.-Y. J. Cancer Mol. 2006, 2, 183-190; Uhland, K. Cell. Mol. Life. Sci. 2006, 63, 2968-2978; List, K. Future Oncol. 2009, 5, 97-104.) Unlike most proteases, which are either secreted from or retained in the cell, matriptase, as a TTSP, is readily accessible on the cell surface and hence a good target for a variety of therapies, including vaccines, monoclonal antibodies and small molecule compounds. Inhibition of the enzyme results in concomitant inhibition of two crucial mediators of tumorigenesis, hepatocyte growth factor (HGF) and the urokinase-type plasminogen activator (uPA). HGF and uPA have been implicated in cancer invasion and metastasis for their roles in cellular motility, extracellular matrix degradation and tumor vascularization.

Matriptase activity is regulated by an endogenous agent, hepatocyte growth factor activator inhibitor (HAI-1), an epithelial Kunitz-type transmembrane inhibitor that displays activity against a wide range of trypsin-like serine proteases. (Oberst, M. D.; Chen, L.-Y. L.; Kiyomiya, K.-I.; Williams, C. A.; Lee, M.-S.; Johnson, M. D.; Dickson, R. B.; Lin, C.-Y. Am. J. Physiol. 2005, 289, C462-C470; Kojima, K.; Tsuzuki, S.; Fushiki, T.; Inouye, K. J. Biol. Chem. 2008, 283, 2478-2487.)

Matriptase has been found to play a role in the degradation of the extracellular matrix and promote tumor metastasis. (WO 00/53232; WO 01/97794; WO 02/08392; Hooper, J. Biol. Chem. 2001, 276, 857-860.) This activity is similar to that seen with certain matrix metalloprotease enzymes (MMP), including stromtelysin and type IV collagenase. Reduction in matriptase-1 expression has been associated with a reduction in the aggressive nature and progression of prostate cancer in a mouse model. (Sanders, A. J.; Parr, C.; Davies, G.; et al. J. Exp. Ther. Oncol. 2006, 6, 39-48.)

Additionally, matriptase plays a role in a pericellular proteolytic pathway responsible for general epithelial homeostasis and in terminal epidermal differentiation. (List, K.; Kosa, P.; Szabo, R.; et al. Am. J. Pathol. 2009, 175, 1453-1463.) Matriptase also induces release of inflammatory cytokines in endothelial cells through activation of PAR-2. Inhibitors would, therefore, have utility as anti-inflammatory agents. Further, the protease is expressed in monocytes and its interaction with PAR-2 contributes to atherosclerosis. Hence, inhibitors of matriptase also have utility for the treatment and prophylaxis of atherosclerosis. (Seitz, I.; Hess, S.; Schulz, H.; Eckl, R.; Busch, G.; et al. Arterioscler. Throm. Vase. Biol. 2007, 27, 769-775.)

Matriptase gene expression has been found to be significantly enhanced in osteoarthritis and the enzyme is involved in initiating multiple mechanisms that lead to cartilage matrix degradation. (Milner, J. A.; Patel, A.; Davidson, R. K.; et al. Arthr. Rheum. 2010, 62, 1955-1966.) Inhibition of the enzyme therefore would be an approach to therapy for this indication.

Matriptase-2 (TMPRSS6) is a TTSP expressed by the liver. (WO 2008/009895; Ramsay, A. J.; Reid, J. C.; Velasco, G.; Quigley, J. P.; Hooper, J. D. Front. Biosci. 2008, 13, 569-579.) Matriptase-2 acts in normal situations to downregulate hepicidin, a hormone that inhibits iron absorption in the intestine and iron release from macrophages. Mutations in the gene for this enzyme lead to aberrant proteolytic activity in humans that has been associated with iron-refractory iron deficiency anemia (IRIDA) due to elevated hepcidin levels. (Folgueras, A. R.; Martin de Lara, F.; Pendas, A. M.; Garabaya, C.; et al. Blood 2008, 112, 2539-3545; Anderson, G. J.; Frazer, D. M.; McLaren, G. D. Curr. Opin. Gastroenterol. 2009, 25, 129-135; Ramsay, A. J.; Hooper, J. D.; Folgueras, A. R.; Velasco, G.; Lopez-Otin, C. Haematologica 2009, 94, 840-849; Finberg, K. E. Semin. Hematol. 2009, 46, 378-386; Cui, Y.; Wu, Q.; Zhou, Y. Kidney Intl. 2009, 76, 1137-1141; Lee, P. Acta Haematologica 2009, 122, 87-96; deFalco, L.; Totaro, F.; Nai, A.; et al. Human Mut. 2010, 31, e1390-e1405.) This enzyme has 35% sequence homology to matriptase-1.

In contrast to the actions of matriptase-1, matriptase-2 inhibits breast tumor growth and invasion with plasma levels correlating with favorable prognosis. (Parr, C.; Sanders, A. J.; Davies, G.; et al. Clin. Cancer Res. 2007, 13, 3568-3576.) The role of this enzyme in cancer development and progression and the potential for modulation as a therapeutic approach remains active areas of study. (Sanders, A. J.; Webb, S. L.; Parr, C.; Mason, M. D.; Jiang, W. G. Anti-cancer Agents Med. Chem. 2010, 10, 64-69.). Matriptase-2 and derived agents also have been reported as a treatment for prostate cancer (WO 2009/009895).

Matriptase-3 is conserved in many species and displays broad serpin activity, but with an expression pattern and regulatory network unique from other TTSP. (Szabo, R.; Netzel-Amett, S.; Hobson, J. P.; Antalis, T. M. Bugge, T. H. Biochem. J. 2005, 390, 231-242.)

In addition to the matriptase enzymes, other TTSP include, but are not limited to, pepsin (TMPRSS1), TMPRSS2, TMPRSS3/TADG-12, TMPRSS4, mosaic serine protease large form (MSPL), TMPRSS11A, human airway trypsin-like protease (HAT), HAT-like 2, HAT-like 3, HAT-like 4, HAT-like 5, polyserase-1, spinesin, enteropeptidase, corin and differentially expressed in squamous cell carcinoma 1 (DESC1). Mutations in TTSP genes have been established as the underlying cause of several genetic disorders in humans and altered expression of TTSP genes are relevant to human carcinogenesis.

Proteases are also involved in causing a variety of deleterious skin conditions. They play a role in both epidermal differentiation (Zeeuwen, P. L. J. M.; Eur. J. Cell Biol. 2004, 83, 761-773) and epithelial development (Bugge, T. H.; List, K.; Szabo, R. Front. Biosci. 2007, 12, 5060-5070). Signaling cascades involving serine proteases play a critical role in epidermal homeostasis. (Ovaere, P.; Lippens; S.; Vandenabeele, P.; Declercq, W. Trends Biochem. Sci. 2009, 34, 453-463.) In addition to matriptase-1, these include furin, prostasin, kallikrein-related peptidase 4 (KLK4, prostate), stratum corneum tryptic enzyme (SCTE, kallikrein-related peptidase 5, KLK5), kallikrein-related peptidase 6 (KLK6, protease M), stratum corneum chymotryptic enzyme (SCCE, kallikrein-related peptidase 7, KLK7), kallikrein-related peptidase 8 (KLK8, neuropsin), kallikrein-related peptidase 10 (KLK10), kallikrein-related peptidase 11 (KLK11), kallikrein-related peptidase 13 (KLK13), kallikrein-related peptidase 14 (KLK14). For example, the involvement of a pro-kallikrein pathway activated by matriptase in disease onset has been identified in a mouse model of Netherton syndrome. (Sales, K. U.; Masedunskas, A.; Bey, A. L.; et al. Nat. Genetics 2010, 42, 676-683.) These protease enzymes elicit an inflammatory response when they begin to break down the protective tissues comprising skin layers. In addition, changes in the proteolytic balance in the skin can result in inflammation leading to redness, scaling and itching. Indeed, proteases, their inhibitors and their target proteins, including flaggrin, protease-activated receptors (PAR) and corneodesmosin, comprise a regulatory network for skin tissues and contribute to the integrity and barrier functions of the skin. (Meyer-Hoffert, U. Arch. Immunol. Ther. Exp. 2009, 57, 345-354.) Inhibitors would be useful in reducing these inflammatory events and treating a variety of skin and tissue disorders.

In addition to the skin, matriptase plays a key role in regulating epithelial bather formation and permeability in the intestine. (Buzza, M. S.; Netzel-Arnett, S.; Shea-Donohue, T.; et al. Proc. Nat. Acad. Sci. 2010, 107, 4200-4205.)

Proteases also are responsible for the regulation of epithelial sodium channels (ENaC). (Planes, C.; Caughey, G. H.; Curr. Top. Development. Biol. 2007, 78, 23-46; Frateschi, S.; Charles, R.-P.; Hummler, E. Open Derm. 2010, 4, 2T35.) Channel activating proteases (CAP) involved in modulating ENaC include prostasin (CAPI, PRSS8), PRSS22, TMPRSS11B, TMPRSS11E, TMPRSS2, TMPRSS3, TMPRSS4 (MT-SP2), MT-SP1, CAP2, CAP3, trypsin, cathepsin A and neutrophil elastase. Inhibitors of CAP have been disclosed, with chemical structures based around a pyrrolidine basic scaffold as shown (WO 2007/137080; WO 2007/140117; WO 2008/085608; WO 2008/097673; WO 2008/097676).

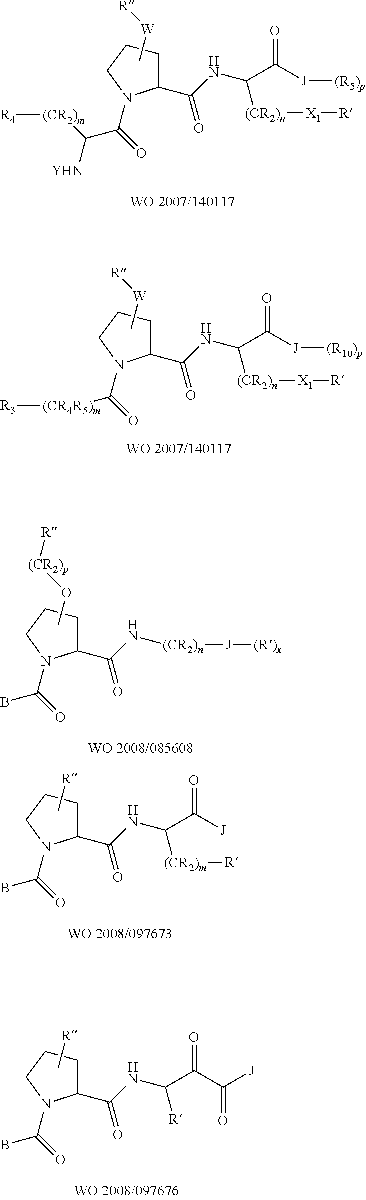

To date, only a limited number of inhibitors of matriptase have been described. These include small molecules such as meta-substituted sulfonyl amides of secondary amino acid amides (WO 2008/107176; Steinmetzer, T.; Doennecke, D.; Korsonewski, M.; Neuwirth, C.; Steinmetzer, P.; Schulze, A.; Saupe, S. M.; Schweinitz, A. Bioorg. Med. Chem. Lett. 2009, 19, 67-73; Schweinitz, A.; Doennecke, D.; Ludwig, A.; Steinmetzer, P.; Schulze, A.; Kotthaus, J.; Wein, S.; Clement, B.; Steinmetzer, T. Bioorg. Med. Chem. Lett. 2009, 19, 1960-1965.)

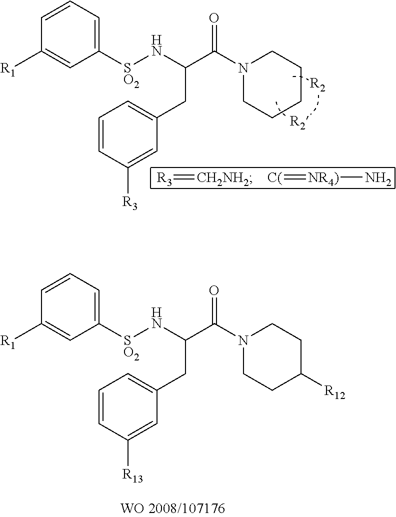

Another structural class of matriptase inhibitors is based upon N-sulfonylated amino acid derivatives (WO 2004/101507; US 2007/0055065; Steinmetzer, T.; Schweinitz, A.; Stuerzbecher, A.; et al. J. Med. Chem. 2006, 49, 4116-4126).

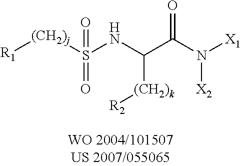

Linear peptide (U.S. Pat. No. 6,797,504; U.S. Pat. No. 7,157,596; WO 02/020475) and peptidomimetic (U.S. Pat. No. 7,019,019; WO 2004/058688) inhibitors have been disclosed.

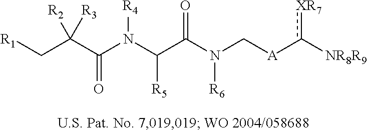

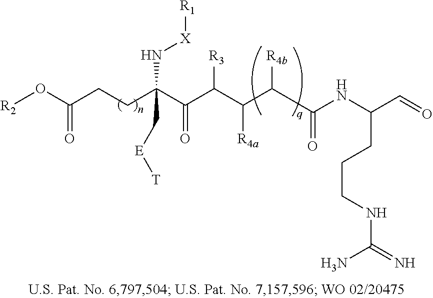

One of these peptidomimetic matriptase inhibitors, CVS-3983, has shown activity in an in vivo model of tumor metastasis. (Gallein, A. V.; Mullen, L.; Fox, W. D.; Brown, J.; et al. Prostate 2004, 61, 228-235.)

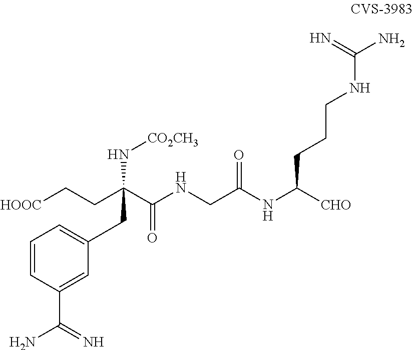

Studies on the metabolism and distribution of two other peptidomimetic inhibitors, CJ-1737 and CJ-672, have revealed important differences in metabolism between animals and humans for these types of molecules. (Kotthaus, J.; Steinmetzer, T.; Kotthaus, J.; Schade, D.; van de Locht, A.; Clement, B. Xenobiotica 2010, 40, 93-101.)

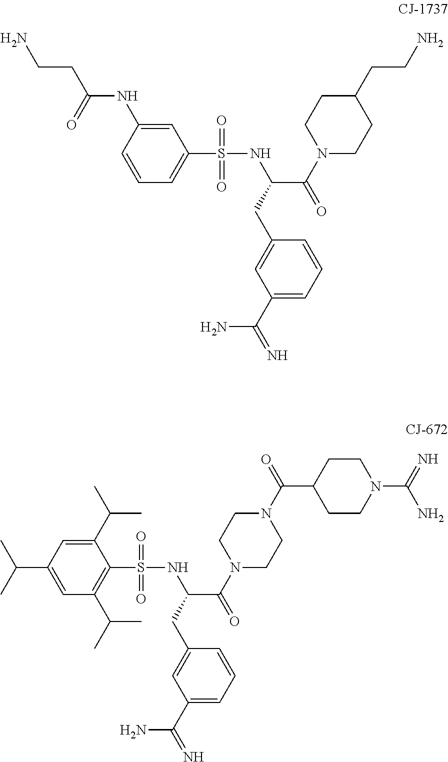

More recently, N-protected dipeptides containing a 4-amidinobenzylamide have been reported as matriptase-1 and matriptase-2 inhibitors. (Sisay, M. T.; Steinmetzer, T.; Stirnberg, M.; Maurer, E.; Hammami, M.; Bajorath, J.; Guetschow, M. J. Med. Chem. 2010, 53, 5523-5535.) Compound 1 displayed 50-fold selectivity for inhibition of matriptase-1 over matriptase-2. These first small molecule inhibitors of matriptase-2 are suggested as possible therapeutics for treatment of iron disorders such as hemochromatosis and iron loading anemias where the level of hepcidin is too low.

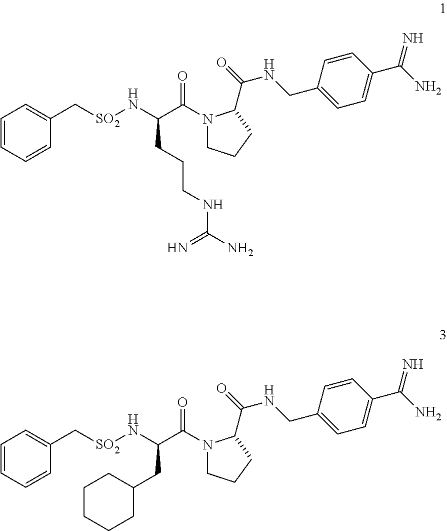

Longer linear peptides, which are eglin c variants, also are known as matriptase inhibitors. (Desilets, A.; Longpre, J.-M.; Beaulieu, M.-E.; Leduc, R. FEBS Lett. 2006, 580, 222T2232.)

Sunflower trypsin inhibitor (SFTI-1), a bicyclic peptide with 14 amino acid residues, has been identified as an inhibitor of matriptase, as well as cathepsin G. This inhibitor has selectivity versus other protease enzymes, including elastase, thrombin and Factor Xa. (Luckett, J. Mol. Biol. 1999, 290, 525.) Unfortunately, SFTI-1 is relatively rapidly degraded in vivo and does not exhibit selectivity over the important physiological serine proteases, trypsin and chymotrypsin, thereby rendering it unsuitable for use as a pharmaceutical agent.

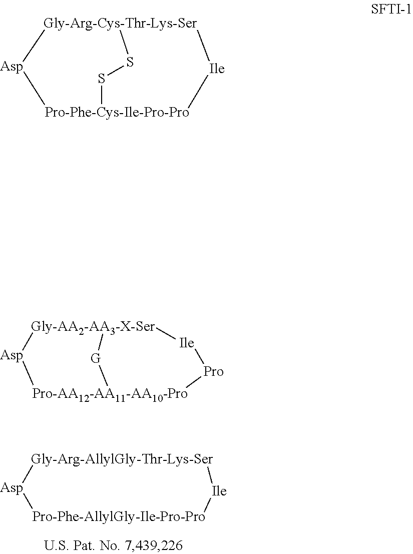

SFTI-1 analogues and mimetics, also bicyclic in nature, have been reported. (U.S. Pat. No. 7,439,226; WO 2006/043933; Long, Y.-Q.; Lee, S.-L.; Lin, C.-Y.; Enyedy, I. J.; Wang, S.; Li, P.; Dickson, R. B.; Roller, P. P. Bioorg. Med. Chem. Lett. 2001, 11, 2515-2519; Jiang, S.; Li, P.; Lee, S.-L. L.; Lin, C.-Y.; Long, Y.-Q.; Johnson, M. D.; Dickson, R. B. Roller, P. B. Org. Lett. 2007, 9, 9-12; Li, P.; Jiang, S.; Lee, S.-L. L.; Lin, C.-Y.; Johnson, M. D.; Dickson, R. B.; Michejda, C. J.; Roller, P. J. J. Med. Chem. 2007, 50, 5976-5983.)

Cyclic peptides containing either 11 or 14 amino acids and methods of use for the prevention or treatment of skin irritation, which act by inhibition of serine proteases, including matriptase, were disclosed in U.S. Pat. No. 7,217,690.

Natural and synthetic protease inhibitors (Yamasaki, Y.; Satomi, S.; Murai, N.; Tsuzuki, A.; Fushiki, T. J. Nutr. Sci. Vitamin. 2003, 49, 27-32), as well as synthetic Kunitz-type inhibitors (WO 2007/079096), have displayed activity against multiple protease enzymes including matriptase.

Indeed, within a particular class of proteases, the enzymes interact with their substrates using common chemical and structural features and, hence, inhibitors can often inhibit other enzymes within the class as well. Of course, when selectivity between enzymes is important, such as to limit specific side effects, this also creates a challenge that must be overcome.

A series of matriptase inhibitors with linear structures separating two or more key basic interacting moieties, such as amidines or the alternatives shown resulting from a structure-based design have been reported (U.S. Pat. No. 6,677,377; WO 01/097784; Enyedy, I. J.; Lee, S.-L.; Kuo, A. H.; Dickson, R. B.; Lin, C.-Y.; Wang, S. J. Med. Chem. 2001, 44, 1349-1355). In these compounds, Z represents either a linear chain of carbon atoms, optionally substituted with one or more oxygen or sulfur atoms, or an aromatic or heteroaromatic spacer component.

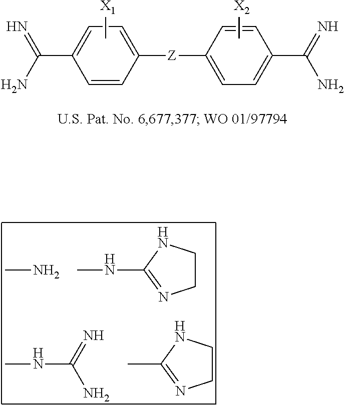

Human monoclonal antibodies directed against matriptase have been disclosed for the diagnosis, prophylaxis or treatment of cancer. (U.S. Pat. No. 7,572,444; WO 2006/068975; Farady, C. J.; Sun, J.; Derragh, M. R.; Miller, S. M.; Craik, C. S. J. Mol. Biol. 2007, 369, 1041-1051; Farady, C. J.; Egea, P. F.; Schneider, E. L.; Darragh, M. R.; Craik, C. S. J. Mol. Biol. 2008, 380, 351-360.) Other antibodies, derived from the matriptase protein, for use in treatment, screening, diagnosis, prognosis and therapy of various types of cancer have also been described (WO 2009/020645; US 2003/270245; US 2009/0155248), as have matriptase murine antibodies (U.S. Pat. No. 7,355,015). Antibody kits for the detection of matriptase are the subject of U.S. Pat. No. 7,022,821.

Antigenic peptides comprising partial sequences of matriptase and other cancer-associated proteases that could be used to generate antibodies for diagnostic or therapeutic purposes are provided in WO 2008/066749.

Agents that stimulate matriptase expression have been disclosed as useful for cosmetic purposes (WO 2008/034821).

To date no matriptase inhibitors have reached clinical development, so there remains a need for new matriptase inhibitors with different structures than those already investigated to be pursued as pharmacological agents.

### SUMMARY OF THE INVENTION

The present invention provides novel conformationally-defined macrocyclic compounds. These compounds can function as modulators, in particular inhibitors, of serine protease enzymes.

According to aspects of the present invention, the present invention relates to a compound according to formula (I):

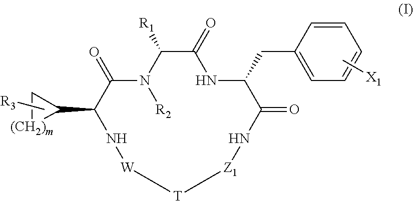

and pharmaceutically acceptable salts thereof
   wherein:

R₁ is selected from the group consisting of —H, —CH₃, —CH₂CH₃, —(CH₂)₂CH₃ and —CH(CH₃)₂;

R₂ is selected from the group consisting of —H, —CH₃ and —CH₂CH₃;

R₃ is optionally present and is selected from the group consisting of C₁-C₄ alkyl, hydroxyl and alkoxy;

m is 1, 2, 3, 4 or 5;

X₁ is selected from the group consisting of amidino, ureido and guanidino;

W is selected from the group consisting of CR₄ₐR_{4b}, wherein R₄ₐ and R_{4b} are independently selected from the group consisting of hydrogen, C₁-C₄ alkyl and trifluoromethyl;

Z₁ is selected from the group consisting of CR₅ₐR_{5b}, wherein R₅ₐ and R_{5b} are independently selected from the group consisting of hydrogen, C₁-C₄ alkyl and trifluoromethyl; and

T is selected from the group consisting of:

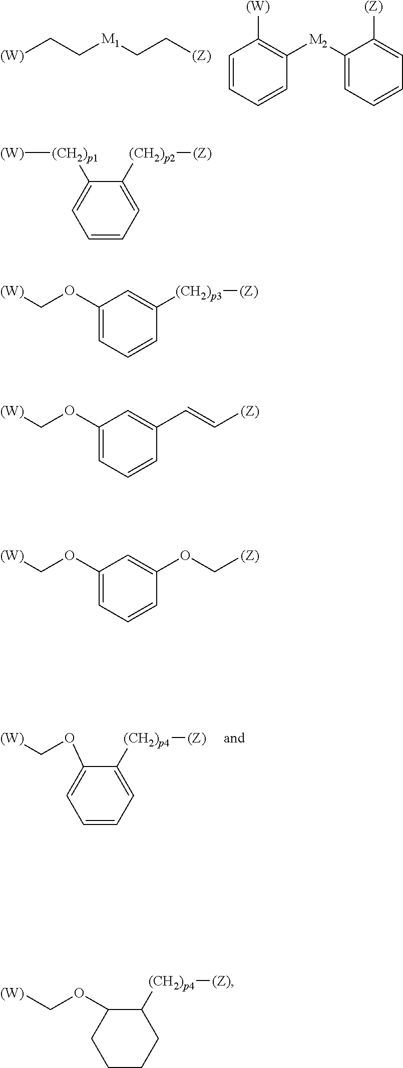

wherein M₁ is selected from the group consisting of O and (CH₂)_{q}, wherein q is 1, 2, 3, 4 or 5; M₂ is selected from the group consisting of O, S, NR₆ and CR₇ₐR_{7b}, wherein R₆ is selected from the group consisting of hydrogen, alkyl, formyl, acyl, carboxyalkyl, carboxyaryl, amido, sulfonyl and sulfonamido; R₇ₐ and R_{7b} are independently selected from the group consisting of hydrogen, hydroxyl, alkoxy, C₁-C₄ alkyl and trifluoromethyl; p1 and p2 are independently 0, 1, 2 or 3; and p3, p4 and p5 are independently 0, 1 or 2.

(W) indicates the site of bonding to the attached carbon atom of W.

(Z) indicates the site of bonding to the attached carbon atom of Z₁.

Additional aspects of the present invention relate to a compound according to formula (II):

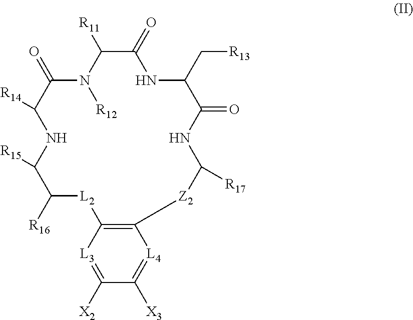

or a pharmaceutically acceptable salt thereof, wherein:
R₁₁ is selected from the group consisting of —H, —CH₂CH₃, —(CH₂)₂CH₃ and —CH(CH₃)₂;
R₁₂ is selected from the group consisting of —H, —CH₃ and —CH₂CH₃;
R₁₃ is selected from the group consisting of —(CH₂)ᵣ₁NR₁₈ₐR_{18b}, —(CH₂)ᵣ₂CONR₁₉ₐR_{19b},

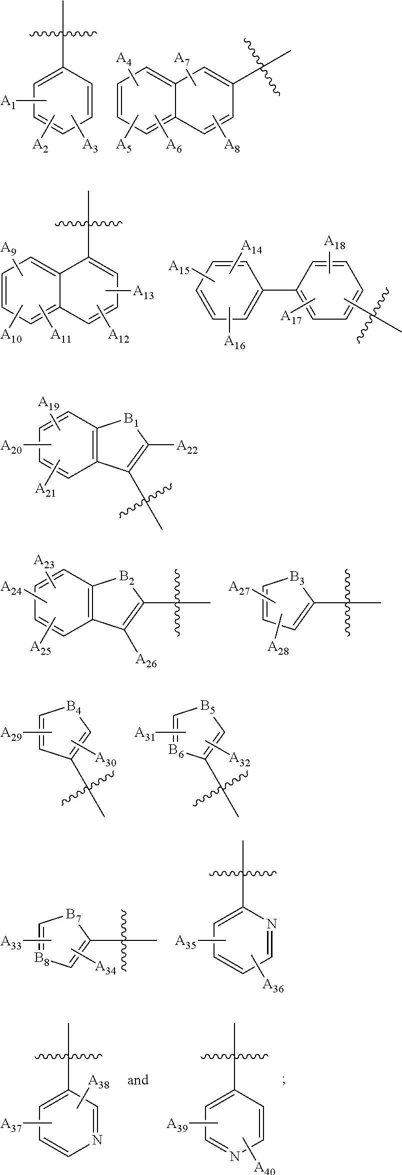

wherein r1 is 1, 2, 3, 4 or 5; r2 is 1, 2 or 3; R₁₈ₐ, R₁₉ₐ and R_{19b} are independently selected from the group consisting of hydrogen and C₁-C₄ alkyl; R_{18b} is selected from the group consisting of hydrogen, C₁-C₄ alkyl, formyl, acyl, amido, amidino and sulfonamido; A₁, A₄, A₇, A₉, A₁₂, A₁₄, A₁₇, A₁₉, A₂₃, A₃₅, A₃₇ and A₃₉ are each optionally present and are independently selected from the group consisting of halogen, trifluoromethyl, amidino, ureido, guanidino, hydroxyl, alkoxy and C₁-C₄ alkyl; A₂, A₃, A₅, A₆, A₈, A₁₀, A₁₁, A₁₃, A₁₅, A₁₆, A₁₈, A₂₀, A₂₁, A₂₄, A₂₅, A₃₆, A₃₈ and A₄₀ are each optionally present and are independently selected from the group consisting of halogen, trifluoromethyl, hydroxyl, alkoxy and C₁-C₄ alkyl; A₂₂, A₂₆, A₂₇, A₂₉, A₃₁ and A₃₃ are each optionally present and are independently selected from the group consisting of trifluoromethyl, amidino, ureido, guanidino and C₁-C₄ alkyl; A₂₈, A₃₀, A₃₂ and A₃₄ are each optionally present and are independently selected from the group consisting of trifluoromethyl and C₁-C₄ alkyl; and B₁, B₂, B₃, B₄, B₅ and B₇ are independently NR₂₀, S or O, wherein R₂₀ is selected from the group consisting of hydrogen, alkyl, formyl, acyl, carboxyalkyl, carboxyaryl, amido, sulfonyl and sulfonamido; and B₆ and B₈ are independently N or CH;

R₁₄ is selected from the group consisting of C₁-C₄ alkyl, optionally substituted with amino, hydroxyl, alkoxy, carboxy, ureido, amidino, or guanidine, and C₃-C₇ cycloalkyl, optionally substituted with alkyl, hydroxyl or alkoxy;

R₁₅ and R₁₆ are independently selected from the group consisting of hydrogen, C₁-C₄ alkyl, hydroxyl and alkoxy;

R₁₇ is selected from the group consisting of hydrogen and C₁-C₄ alkyl;

n is 1, 2, 3, 4 or 5;

Z₂ is selected from the group consisting of CHR₂₁ₐCHR₂₂ₐ, CR_{21b}═CR_{22b}, and C≡C, wherein R₂₁ₐ and R₂₂ₐ are independently selected from the group consisting of hydrogen, C₁-C₄ alkyl, hydroxyl and alkoxy; or R₂₁ₐ and R₂₂ₐ together with the carbons to which they are bonded form a three-membered ring; and R_{21b} and R_{22b} are independently selected from the group consisting of hydrogen and C₁-C₄ alkyl;

X₂ is selected from the group consisting of hydrogen, halogen, amidino, ureido and guanidino;

X₃ is selected from the group consisting of hydrogen, hydroxyl, alkoxy, amino, halogen, trifluoromethyl and C₁-C₄ alkyl;

L₂ is selected from the group consisting of O and CR₂₃ₐR_{23b}, wherein R₂₃ₐ is selected from the group consisting of hydrogen, C₁-C₄ alkyl, hydroxyl and alkoxy; and R_{23b} is selected from the group consisting of hydrogen and C₁-C₄ alkyl;

L₃ is selected from the group consisting of CX₄ and N, wherein X₄ is selected from the group consisting of hydrogen, halogen, hydroxyl, alkoxy, amino, halogen, trifluoromethyl, amidino, ureido and guanidino; and

L₄ is selected from the group consisting of CX₅ and N, wherein X₅ is selected from the group consisting of hydrogen, halogen, trifluoromethyl, hydroxyl, alkoxy, amino, amidino, ureido and guanidino.

The novel macrocyclic compounds of the present invention are useful as modulators, in particular inhibitors, of serine protease enzymes. A number of different cancers can be addressed by these inhibitors, in particular those characterized by tumor metastasis. In addition, inhibitors of serine proteases such as compounds of the present invention can be utilized for the treatment or prevention of skin disorders, such as atopic dermatitis, rosacea, psoriasis, ichthyosis, follicular atrophoderma, hyperkeratosis, hypotrichosis, Netherton syndrome and others.

In particular embodiments of the invention, the serine protease enzyme is matriptase-1 (MTSP-1, ST14, TADG-15, epithin), matriptase-2 (TMPRSS6), matriptase-3, MTSP-4, MTSP-6, MTSP-7, MTSP-9, MTSP-10, PRSS22, TMPRSS11A, TMPRSS11C, TMPRSS2, TMPRSS3, TMPRSS4, TMPRSS5 (spinesin), mosaic serine protease large form (MSPL), enteropeptidase, polyserase-1, corin, human airway trypsin-like protease (HAT), HAT-like 2, HAT-like 3, HAT-like 4, HAT-like 5, prostasin (CAP1, PRSS8), CAP2, CAP3, trypsin, cathepsin A, neutrophil elastase, hepsin, stratum corneum tryptic enzyme (SCTE, kallikrein-related peptidase 5, KLK5), stratum corneum chymotryptic enzyme (SCCE, kallikrein-related peptidase 7, KLK7), kallikrein-related peptidase 4 (KLK4, prostase), kallikrein-related peptidase 8 (KLK8, neuropsin), kallikrein-related peptidase 11 (KLK11), kallikrein-related peptidase 13 (KLK13), kallikrein-related peptidase 14 (KLK14), kallikrein-related peptidase 6 (KLK6, protease M), kallikrein-related peptidase 10 (KLK10), granzyme B, calcium signal transducer 1, calcium signal transducer 2, claudin 3, claudin 4, Turin, ladinin, larninin, plasmin, stratifin, SI00A2, CD24, lipocalin 2, osteopontin, tissue-type plasminogen activator, urokinase-type plasminogen activator or differentially expressed in squamous cell carcinoma 1 (DESC1).

Compounds of the present invention are also useful for pathological conditions characterized by abnormal neovascularization or angiogenesis. Examples of such conditions include, but are not limited to, ocular neovascular disease, hemangioma and disorders characterized by chronic inflammation, including rheumatoid arthritis and Crohn's disease.

In other aspects of the present invention, compounds of the invention can be used to treat pathological conditions characterized by deregulated iron homeostasis including in particular embodiments, iron-refractory iron deficiency anemia (IRIDA), systemic iron overload (hemochromatosis) or iron loading anemia.

Further aspects of the present invention further provide pharmaceutical compositions comprising a compound of formula (I) or a compound of formula (II) and a pharmaceutically acceptable carrier, excipient or diluent.

Other aspects of the present invention provide methods of treating a hyperproliferative disorder, inflammatory disorder, tissue disorder, cardiovascular disorder, respiratory disorder or viral infection, including administering to a subject in need thereof an effective amount of a compound of formula (I) or formula (I).

Additional aspects of the present invention provide kits comprising one or more containers containing pharmaceutical dosage units comprising an effective amount of one or more compounds of the present invention packaged with optional instructions for the use thereof.

Further aspects of the present invention relate to methods of making the compounds of formula (I) and formula (II).

Aspects of the present invention further relate to methods of preventing and/or treating disorders described herein, in particular, pathological conditions, hyperproliferative disorders, tissue disorders, inflammatory disorders, respiratory disorders and viral infections.

In particular embodiments, the hyperproliferative disorder is leukemia, including CML, lymphoma, breast cancer, gastrointestinal cancer, esophageal cancer, stomach cancer, gastric cancer, colon cancer, bowel cancer, colorectal cancer, prostate cancer, bladder cancer, testicular cancer, ovarian cancer, uterine cancer, cervical cancer, endometrial cancer, epithelial cancer, head and neck cancer, brain cancer, lung cancer, liver cancer, renal cancer, bronchial cancer, pancreatic cancer, thyroid cancer, bone cancer and skin cancer.

In other particular embodiments, the hyperproliferative disorder is characterized by tumor metastasis, wherein the tumor is found in the breast, brain, ovary, colon, rectum, stomach, liver, kidney, intestine, mouth, throat, esophagus, prostate, testes, bladder, uterus, cervix, lung, pancreas, bone, thyroid or skin.

In other specific embodiments, the hyperproliferative disorder is prostate adenocarcinoma, ovarian carcinoma, cervical neoplasia, small cell lung cancer, non-small cell lung cancer, renal cell carcinoma, pancreatic ductal adenocarcinoma, uterine leiomyosarcoma, transitional cell carcinoma, nonmelanoma skin cancer, squamocellular carcinoma, malignant mesothelioma or glioblastoma.

In additional embodiments, compounds of the present invention can be used for the treatment or prevention of tissue or skin disorders, including in particular embodiments, atopic dermatitis, rosacea, psoriasis, ichthyosis, follicular atrophoderma, hyperkeratosis, hypotrichosis, Netherton syndrome and others.

In still other particular embodiments, the inflammatory disorder is rheumatoid arthritis, osteoarthritis, Crohn's disease, ulcerative colitis or atherosclerosis.

In further particular embodiments, the pathological condition is characterized by epithelial cell proliferation or abnormal neovascularization.

In additional particular embodiments, the respiratory disorder is cystic fibrosis, bronchitis, chronic obstructive pulmonary disease (COPD), asthma, allergic rhinitis, ciliary dyskinesia, lung carcinoma, pneumonia or a respiratory infection.

In still other particular embodiments, the viral infection is caused by influenza viruses or metapneumovirus.

The present invention also relates to compounds of formula (I) or (II) used for the preparation of a medicament for prevention and/or treatment of the disorders described herein.

The foregoing and other aspects of the present invention are explained in greater detail in the specification set forth below.

### BRIEF DESCRIPTION OF THE DRAWINGS

FIG. 1 shows a reaction scheme for the synthesis of a representative compound of the present invention.

FIG. 2 shows a reaction scheme for the simultaneous synthesis of multiple representative compounds of the present invention.

FIG. 3 shows another reaction scheme for the simultaneous synthesis of multiple representative compounds of the present invention.

FIG. 4 shows a reaction scheme for the synthesis of tether T32.

FIG. 5 shows a reaction scheme for the synthesis of tether T201.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this invention belongS.

All publications, U.S. patent applications, U.S. patents and other references cited herein are incorporated by reference in their entireties.

The term “alkyl” refers to straight or branched chain saturated or partially unsaturated hydrocarbon groups having from 1 to 20 carbon atoms, in some instances 1 to 8 carbon atoms. The term “lower alkyl” refers to alkyl groups containing 1 to 6 carbon atoms. Examples of alkyl groups include, but are not limited to, methyl, ethyl, isopropyl, tert-butyl, 3-hexenyl, and 2-butynyl. By “unsaturated” is meant the presence of 1, 2 or 3 double or triple bonds, or a combination of the two. Such alkyl groups may also be optionally substituted as described below.

When a subscript is used with reference to an alkyl or other hydrocarbon group defined herein, the subscript refers to the number of carbon atoms that the group may contain. For example, C₂-C₄ alkyl indicates an alkyl group with 2, 3 or 4 carbon atoms.

The term “cycloalkyl” refers to saturated or partially unsaturated cyclic hydrocarbon groups having from 3 to 15 carbon atoms in the ring, in some instances 3 to 7, and to alkyl groups containing said cyclic hydrocarbon groups. Examples of cycloalkyl groups include, but are not limited to, cyclopropyl, cyclopropylmethyl, cyclopentyl, 2-(cyclohexyl)ethyl, cycloheptyl, and cyclohexenyl. Cycloalkyl as defined herein also includes groups with multiple carbon rings, each of which may be saturated or partially unsaturated, for example decalinyl, [2.2.1]-bicycloheptanyi or adamantanyl. All such cycloalkyl groups may also be optionally substituted as described below.

The term “aromatic” refers to an unsaturated cyclic hydrocarbon group having a conjugated pi electron system that contains 4n+2 electrons where n is an integer greater than or equal to 1. Aromatic molecules are typically stable and are depicted as a planar ring of atoms with resonance structures that consist of alternating double and single bonds, for example benzene or naphthalene.

The term “aryl” refers to an aromatic group in a single or fused carbocyclic ring system having from 6 to 15 ring atoms, in some instances 6 to 10, and to alkyl groups containing said aromatic groups. Examples of aryl groups include, but are not limited to, phenyl, 1-naphthyl, 2-naphthyl and benzyl. Aryl as defined herein also includes groups with multiple aryl rings which may be fused, as in naphthyl and anthracenyl, or unfused, as in biphenyl and terphenyl. Aryl also refers to bicyclic or tricycle carbon rings, where one of the rings is aromatic and the others of which may be saturated, partially unsaturated or aromatic, for example, indanyl or tetrahydronaphthyl (tetralinyl). All such aryl groups may also be optionally substituted as described below.

The term “heterocycle” or “heterocyclic” refers to saturated or partially unsaturated monocycle, bicyclic or tricyclic groups having from 3 to 15 atoms, in some instances 3 to 7, with at least one heteroatom in at least one of the rings, said heteroatom being selected from O, S or N. Each ring of the heterocyclic group can contain one or two O atoms, one or two S atoms, one to four N atoms, provided that the total number of heteroatoms in each ring is four or less and each ring contains at least one carbon atom. The fused rings completing the bicyclic or tricyclic heterocyclic groups may contain only carbon atoms and may be saturated or partially unsaturated. The N and S atoms may optionally be oxidized and the N atoms may optionally be quaternized. Heterocyclic also refers to alkyl groups containing said monocyclic, bicyclic or tricyclic heterocyclic groups. Examples of heterocyclic rings include, but are not limited to, 2- or 3-piperidinyl, 2- or 3-piperazinyl, 2- or 3-morpholinyl. All such heterocyclic groups may also be optionally substituted as described below

The term “heteroaryl” refers to an aromatic group in a single or fused ring system having from 5 to 15 ring atoms, in some instances 5 to 10, which have at least one heteroatom in at least one of the rings, said heteroatom being selected from O, S or N. Each ring of the heteroaryl group can contain one or two O atoms, one or two S atoms, one to four N atoms, provided that the total number of heteroatoms in each ring is four or less and each ring contains at least one carbon atom. The fused rings completing the bicyclic or tricyclic groups may contain only carbon atoms and may be saturated, partially unsaturated or aromatic. In structures where the lone pair of electrons of a nitrogen atom is not involved in completing the aromatic pi electron system, the N atoms may optionally be quaternized or oxidized to the N-oxide. Heteroaryl also refers to alkyl groups containing said cyclic groups. Examples of monocyclic heteroaryl groups include, but are not limited to pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, benzopyranyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to carbazolyl, benzindolyl, phenanthrollinyl, acridinyl, phenanthridinyl, and xanthenyl. All such heteroaryl groups may also be optionally substituted as described below.

The term “hydroxy” refers to the group —OH.

The term “alkoxy” refers to the group —ORₐ, wherein Rₐ is alkyl, cycloalkyl or heterocyclic. Examples include, but are not limited to methoxy, ethoxy, tert-butoxy, cyclohexyloxy and tetrahydropyranyloxy.

The term “aryloxy” refers to the group —OR_{b} wherein R_{b} is aryl or heteroaryl. Examples include, but are not limited to phenoxy, benzyloxy and 2-naphthyloxy.

The term “acyl” refers to the group —C(═O)—R_{c} wherein R_{c} is alkyl, cycloalkyl, heterocyclic, aryl or heteroaryl. Examples include, but are not limited to, acetyl, benzoyl and furoyl.

The term “amino acyl” indicates an acyl group that is derived from an amino acid.

The term “amino” refers to an —NR_{d}Rₑ group wherein R_{d} and Rₑ are independently selected from the group consisting of hydrogen, alkyl, cycloalkyl, heterocyclic, aryl and heteroaryl. Alternatively, R_{d} and Rₑ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amido” refers to the group —C(═O)—NR_{f}R_{g} wherein R_{f} and R_{g} are independently selected from the group consisting of hydrogen, alkyl, cycloalkyl, heterocyclic, aryl and heteroaryl. Alternatively, R_{f} and R_{g} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amidino” refers to the group —C(═NRₕ)NRᵢRⱼ wherein Rₕ is selected from the group consisting of hydrogen, alkyl, cycloalkyl, heterocyclic, aryl and heteroaryl; and Rᵢ and Rⱼ are independently selected from the group consisting of hydrogen, alkyl, cycloalkyl, heterocyclic, aryl and heteroaryl. Alternatively, Rᵢ and Rⱼ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carboxy” refers to the group —CO₂H.

The term “carboxyalkyl” refers to the group —CO₂Rₖ, wherein Rₖ is alkyl, cycloalkyl or heterocyclic.

The term “carboxyaryl” refers to the group —CO₂Rₘ, wherein Rₘ is aryl or heteroaryl.

The term “cyano” refers to the group —CN.

The term “formyl” refers to the group —C(═O)H, also denoted —CHO.

The term “halo,” “halogen” or “halide” refers to fluoro, fluorine or fluoride, chloro, chlorine or chloride, bromo, bromine or bromide, and iodo, iodine or iodide, respectively.

The term “oxo” refers to the bivalent group ═O, which is substituted in place of two hydrogen atoms on the same carbon to form a carbonyl group.

The term “mercapto” refers to the group —SRₙ wherein Rₙ is hydrogen, alkyl, cycloalkyl, heterocyclic, aryl or heteroaryl.

The term “nitro” refers to the group —NO₂

The term “trifluoromethyl” refers to the group —CF₃.

The term “sulfinyl” refers to the group —S(═O)Rₚ wherein Rₚ is alkyl, cycloalkyl, heterocyclic, aryl or heteroaryl.

The term “sulfonyl” refers to the group —S(═O)₂—R_{q1} wherein R_{q1} is alkyl, cycloalkyl, heterocyclic, aryl or heteroaryl.

The term “aminosulfonyl” refers to the group —NR_{q2}—S(═O)₂—R_{q3} wherein R_{q2} is hydrogen, alkyl, cycloalkyl, heterocyclic, aryl or heteroaryl; and R_{q3} is alkyl, cycloalkyl, heterocyclic, aryl or heteroaryl.

The term “sulfonamido” refers to the group —S(═O)₂—NRᵣRₛ wherein Rᵣ and Rₛ are independently selected from the group consisting of hydrogen, alkyl, cycloalkyl, heterocyclic, aryl or heteroaryl. Alternatively, Rᵣ and Rₛ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amino, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carbamoyl” refers to a group of the formula —N(Rₜ)—C(═O)—ORᵤ wherein Rₜ is selected from hydrogen, alkyl, cycloalkyl, heterocyclic, aryl or heteroaryl; and Rᵤ is selected from alkyl, cycloalkyl, heterocylic, aryl or heteroaryl.

The term “guanidino” refers to a group of the formula —N(Rᵥ)—C(═NR_{w})—NRₓR_{y} wherein Rᵥ, R_{w}, Rₓ and R_{y} are independently selected from hydrogen, alkyl, cycloalkyl, heterocyclic, aryl or heteroaryl. Alternatively, Rₓ and R_{y} together form a heterocyclic ring or 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “ureido” refers to a group of the formula —N(R_{z})—C(═O)—NRₐₐR_{bb} wherein R_{z}, Rₐₐ and R_{bb} are independently selected from hydrogen, alkyl, cycloalkyl, heterocyclic, aryl or heteroaryl. Alternatively, Rₐₐ and R_{bb} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

The term “substituted” when used with the terms alkyl, cycloalkyl, heterocyclic, aryl and heteroaryl refers to an alkyl, cycloalkyl, heterocyclic, aryl or heteroaryl group having one or more of the hydrogen atoms of the group replaced by substituents independently selected from unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, halo, oxo, mercapto, sulfinyl, sulfonyl, sulfonamido, amidino, carbamoyl, guanidino, ureido and groups of the formulas —NR_{cc}C(═O)R_{dd}, —NRₑₑC(═NR_{ff})R_{gg}, —OC(═O)NRₕₕRᵢᵢ, —OC(═O)Rⱼⱼ, —OC(═O)ORₖₖ, —NRₘₘSO₂Rₙₙ, or —NRₚₚSO₂NR_{qq}Rᵣᵣ wherein R_{cc}, R_{dd}, Rₑₑ, R_{ff}, R_{gg}, Rₕₕ, Rᵢᵢ, Rⱼⱼ, Rₘₘ, Rₚₚ, R_{qq} and Rᵣᵣ are independently selected from hydrogen, unsubstituted alkyl, unsubstituted cycloalkyl, unsubstituted heterocyclic, unsubstituted aryl or unsubstituted heteroaryl; and wherein Rₖₖ and Rₙₙ are independently selected from unsubstituted alkyl, unsubstituted cycloalkyl, unsubstituted heterocyclic, unsubstituted aryl or unsubstituted heteroaryl. Alternatively, R_{gg} and Rₕₕ, Rⱼⱼ and Rₖₖ or Rₚₚ and R_{qq} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N. In addition, the term “substituted” for aryl and heteroaryl groups includes as an option having one of the hydrogen atoms of the group replaced by cyano, nitro or tritluoromethyl.

A substitution is made provided that any atom's normal valency is not exceeded and that the substitution results in a stable compound. Generally, when a substituted form of a group is present, such substituted group is preferably not further substituted or, if substituted, the substituent comprises only a limited number of substituted groups, in some instances 1, 2, 3 or 4 such substituents.

When any variable occurs more than one time in any constituent or in any formula herein, its definition on each occurrence is independent of its definition at every other occurrence. Also, combinations of substituents and/or variables are permissible only if such combinations result in stable compounds.

A “stable compound” or “stable structure” refers to a compound that is sufficiently robust to survive isolation to a useful degree of purity and formulation into an efficacious therapeutic agent.

The term “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “unnatural” or “unusual” refers to the wide selection of non-natural, rare or synthetic amino acids such as those described by Hunt, S. in Chemistry and Biochemistry of the Amino Acids, Barrett, G. C., Ed., Chapman and Hall: New York, 1985.

The term “residue” with reference to an amino acid or amino acid derivative refers to a group of the formula:

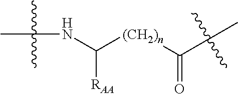

wherein R_{AA} is an amino acid side chain, and n=0, 1 or 2 in this instance.

The term “fragment” with respect to a dipeptide, tripeptide or higher order peptide derivative indicates a group that contains two, three or more, respectively, amino acid residues.

The term “amino acid side chain” refers to any side chain from a standard or unnatural amino acid, and is denoted R_{AA}. For example, the side chain of alanine is methyl, the side chain of valine is isopropyl and the side chain of tryptophan is 3-indolylmethyl.

The term “agonist” refers to a compound that duplicates at least some of the effect of the endogenous ligand of a protein, receptor, enzyme or the like.

The term “antagonist” refers to a compound that inhibits at least some of the effect of the endogenous ligand of a protein, receptor, enzyme or the like.

The term “inhibitor” refers to a compound that reduces the activity of a protein or enzyme.

The term “cancerous condition” is one in which a subject has a progressive cancer such as leukemia, lymphoma, melanoma, breast, gastrointestinal, esophageal, stomach, colon, bowel, colorectal, rectal, prostate, bladder, testicular, ovarian, uterine, cervical, brain, lung, bronchial, larynx, pharynx, pancreatic, thyroid, bone and skin.

The term “channel activating protease” or CAP refers to a membrane anchored protease that is typically secreted on the extracellular membrane of cell, but that can also be secreted into the body and stimulate the activity of the amiloride-sensitive epithelial sodium channel (ENaC). Non-limiting examples of such CAP are prostasin (PRSS**), matriptase, CAP2, CAP3, trypsin, PRSS22, TMPRSS2, TMPRSS 3, TMPRSS4 (matriptase-2), TMPRSS11, cathepsin A, neutrophil elastase and isoforms thereof.

The term “tumor” refers to an abnormal growth of tissue resulting from uncontrolled cell replication. Such abnormal growth is often associated with cancer. A tumor is also referred to as a neoplasm.

The term “metastasis” refers to the spread of cancer or a tumor from an original site to one or more other locations in the body of a subject.

The term “modulates or modulating” refers to imparting an effect on a biological or chemical process or mechanism using a compound. For example, modulating may increase, facilitate, upregulate, activate, inhibit, decrease, block, prevent, delay, desensitize, deactivate, down regulate, or the like, a biological or chemical process or mechanism. Accordingly, a compound that modulates can be an “agonist” or an “antagonist.” Exemplary biological processes or mechanisms affected by modulating include, but are not limited to, receptor activation, binding and/or hormone release or secretion, ion channel regulation, cellular permeability, phosphorylation or dephosphorylation, tissue homeostasis, second messenger signaling and gene regulation. Exemplary chemical processes or mechanisms affected by modulating include, but are not limited to, catalysis and hydrolysis. As used herein, a compound that modulates is termed a “modulator.”

The term “variant” when applied to a receptor is meant to include dimers, trimers, tetramers, pentamers and other biological complexes containing multiple components. These components can be the same or different.

The term “peptide” refers to a chemical compound comprised of two or more amino acids covalently bonded together.

The term “peptidomimetic” refers to a chemical compound designed to mimic a peptide, but which contains structural differences through the addition or replacement of one of more functional groups of the peptide in order to modulate its activity or other properties, such as solubility, metabolic stability, oral bioavailability, lipophilicity, permeability, etc. This can include replacement of the peptide bond, side chain modifications, truncations, additions of functional groups, etc. When the chemical structure is not derived from the peptide, but mimics its activity, it is often referred to as a “non-peptide peptidomimetic.”

The term “peptide bond” refers to the amide [—C(═O)—NH—] functionality with which individual amino acids are typically covalently bonded to each other in a peptide.

The term “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in “Protective Groups in Organic Synthesis,” Theodora W. Greene and Peter G. Wuts, editors, John Wiley & Sons, New York, 3^{rd} edition, 1999 [ISBN 0471160199]. Examples of amino protecting groups include, but are not limited to, phthalimido, trichloroacetyl, benzyloxycarbonyl, tert-butoxycarbonyl, and adamantyloxycarbonyl. In some embodiments, amino protecting groups are carbamate amino protecting groups, which are defined as an amino protecting group that when bound to an amino group forms a carbamate. In other embodiments, amino carbamate protecting groups are allyloxycarbonyl (Alloc or Aloe), benzyloxycarbonyl (Cbz), 9-fluorenylmethoxycarbonyl (Frnoc), tert-butoxycarbonyl (Boc) and α,α-dimethyl-3,5-dimethoxybenzyloxycarbonyl (Ddz). For a recent discussion of newer nitrogen protecting groups: Theodoridis, G. Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to methyl ester, tert-butyl ester, benzyl ester, trimethylsilylethyl ester, and 2,2,2-trichloroethyl ester.

The term “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry.

The term “solid support,” “solid phase” or “resin” refers to a mechanically and chemically stable polymeric matrix utilized to conduct solid phase chemistry. This is denoted by “Resin,” “P—” or the following symbol:

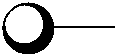

Examples of appropriate polymer materials include, but are not limited to, polystyrene, polyethylene, polyethylene glycol, polyethylene glycol grafted or covalently bonded to polystyrene (also termed PEG-polystyrene, TentaGel™, Rapp, W.; Zhang, L.; Bayer, E. In Innovations and Perspectives in Solid Phase Synthesis. Peptides, Polypeptides and Oligonucleotides; Epton, R., Ed.; SPCC Ltd.: Birmingham, UK; p 205), polyacrylate (CLEAR™), polyacrylamide, polyurethane, PEGA (polyethyleneglycol poly(N,N-dimethylacrylamide) co-polymer, Meldal, M. Tetrahedron Lett. 1992, 33, 3077-3080], cellulose, etc. These materials can optionally contain additional chemical agents to form cross-linked bonds to mechanically stabilize the structure, for example polystyrene cross-linked with divinylbenezene (DVB, usually 0.1-5%, preferably 0.5-2%). This solid support can include as non-limiting examples aminomethyl polystyrene, hydroxymethyl polystyrene, benzhydrylamine polystyrene (BHA), methylbenzhydrylamine (MBNA) polystyrene, and other polymeric backbones containing free chemical functional groups, most typically, —NH₂ or —OH, for further derivatization or reaction. The term is also meant to include “Ultraresins” with a high proportion (“loading”) of these functional groups such as those prepared from polyethyleneimines and cross-linking molecules (Barth, M.; Rademann, J. J. Comb. Chem. 2004, 6, 340-349). At the conclusion of the synthesis, resins are typically discarded, although they have been shown to be able to be reused such as in Frechet, J. M. J.; Haque, K. E. Tetrahedron Lett. 1975, 16, 3055.

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The term “linker” when used in reference to solid phase chemistry refers to a chemical group that is bonded covalently to a solid support and is attached between the support and the substrate typically in order to permit the release (cleavage) of the substrate from the solid support. However, it can also be used to impart stability to the bond to the solid support or merely as a spacer element. Many solid supports are available commercially with linkers already attached.

Abbreviations used for amino acids and designation of peptides follow the rules of the IUPAC-IUB Commission of Biochemical Nomenclature in J. Biol. Chem. 1972, 247, 977-983. This document has been updated: Biochem. 1984, 219, 345-373; Eur. J. Biochem., 1984, 138, 9-37; 1985, 152, 1; Internat. J. Pept. Prot. Res., 1984, 24, following p 84; J. Biol. Chem., 1985, 260, 14-42; Pure Appl. Chem., 1984, 56, 595-624; Amino Acids and Peptides, 1985, 16, 387-410; and in Biochemical Nomenclature and Related Documents, 2nd edition, Portland Press, 1992, pp 39-67. Extensions to the rules were published in the JCBN/NC-IUB Newsletter 1985, 1986, 1989; see Biochemical Nomenclature and Related Documents, 2nd edition, Portland Press, 1992, pp 68-69.

The term “effective amount” or “effective” is intended to designate a dose that causes a relief of symptoms of a disease or disorder as noted through clinical testing and evaluation, patient observation, and/or the like, and/or a dose that causes a detectable change in biological or chemical activity. The detectable changes may be detected and/or further quantified by one skilled in the art for the relevant mechanism or process. As is generally understood in the art, the dosage will vary depending on the administration routes, symptoms and body weight of the patient but also depending upon the compound being administered.

Administration of two or more compounds “in combination” means that the two compounds are administered closely enough in time that the presence of one alters the biological effects of the other. The two compounds can be administered simultaneously (concurrently) or sequentially. Simultaneous administration can be carried out by mixing the compounds prior to administration, or by administering the compounds at the same point in time but at different anatomic sites or using different routes of administration. The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

The term “pharmaceutically active metabolite” is intended to mean a pharmacologically active product produced through metabolism in the body of a specified compound.

The term “solvate” is intended to mean a pharmaceutically acceptable solvate form of a specified compound that retains the biological effectiveness of such compound. Examples of solvates, without limitation, include compounds of the invention in combination with water, isopropanol, ethanol, methanol, DMSO, ethyl acetate, acetic acid, or ethanolamine.

### 1. Compounds

Novel macrocyclic compounds of the present invention include macrocyclic compounds comprising a building block structure including a tether component that undergoes cyclization to form the macrocyclic compound. The building block structure can comprise amino acids (standard and unnatural), hydroxy acids, hydrazino acids, aza-amino acids, specialized moieties such as those that play a role in the introduction of peptide surrogates and isosteres, and a tether component as described herein.

The present invention includes isolated compounds. An isolated compound refers to a compound that, in some embodiments, comprises at least 10%, at least 25%, at least 50% or at least 70% of the compounds of a mixture. In some embodiments, the compound, pharmaceutically acceptable salt thereof or pharmaceutical composition containing the compound exhibits a statistically significant binding and/or antagonist activity when tested in biological assays at the human ghrelin receptor.

In the case of compounds, salts, or solvates that are solids, it is understood by those skilled in the art that the inventive compounds, salts, and solvates may exist in different crystal or polymorphic forms, all of which are intended to be within the scope of the present invention and specified formulas.

The compounds disclosed herein may have asymmetric centers. The inventive compounds may exist as single stereoisomers, racemates, and/or mixtures of enantiomers and/or diastereomers. All such single stereoisomers, racemates, and mixtures thereof are intended to be within the scope of the present invention. In particular embodiments, however, the inventive compounds are used in optically pure form. The terms “S” and “R” configuration as used herein are as defined by the IUPAC 1974 Recommendations for Section E, Fundamentals of Stereochemistry (Pure Appl. Chem. 1976, 45, 13-30).

Unless otherwise depicted to be a specific orientation, the present invention accounts for all stereoisomeric forms. The compounds may be prepared as a single stereoisomer or a mixture of stereoisomers. The non-racemic forms may be obtained by either synthesis or resolution. The compounds may, for example, be resolved into the component enantiomers by standard techniques, for example formation of diastereomeric pairs via salt formation. The compounds also may be resolved by covalently bonding to a chiral moiety. The diastereomers can then be resolved by chromatographic separation and/or crystallographic separation. In the case of a chiral auxiliary moiety, it can then be removed. As an alternative, the compounds can be resolved through the use of chiral chromatography. Enzymatic methods of resolution could also be used in certain cases.

As generally understood by those skilled in the art, an “optically pure” compound is one that contains only a single enantiomer. As used herein, the term “optically active” is intended to mean a compound comprising at least a sufficient excess of one enantiomer over the other such that the mixture rotates plane polarized light. Optically active compounds have the ability to rotate the plane of polarized light. The excess of one enantiomer over another is typically expressed as enantiomeric excess (e.e.). In describing an optically active compound, the prefixes D and L or R and S are used to denote the absolute configuration of the molecule about its chiral center(s). The prefixes “d” and “1” or (+) and (−) are used to denote the optical rotation of the compound (i.e., the direction in which a plane of polarized light is rotated by the optically active compound). The “1” or (−) prefix indicates that the compound is levorotatory (i.e., rotates the plane of polarized light to the left or counterclockwise) while the “d” or (+) prefix means that the compound is dextrarotatory (i.e., rotates the plane of polarized light to the right or clockwise). The sign of optical rotation, (−) and (+), is not related to the absolute configuration of the molecule, R and S.

A compound of the invention having the desired pharmacological properties will be optically active and, can be comprised of at least 90% (80% e.e.), at least 95% (90% e.e.), at least 97.5% (95% e.e.) or at least 99% (98% e.e.) of a single isomer.

Likewise, many geometric isomers of double bonds and the like can also be present in the compounds disclosed herein, and all such stable isomers are included within the present invention unless otherwise specified. Also included in the invention are tautomers and rotamers of the compounds.

The use of the following symbols at the right refers to substitution of one or more hydrogen atoms of the indicated ring with the defined substituent R.

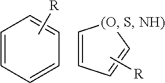

The use of the following symbol indicates a single bond or an optional double bond: [symbol] .

Embodiments of the present invention further provide intermediate compounds formed through the synthetic methods described herein to provide the compounds of formula I and/or II. The intermediate compounds may possess utility as a therapeutic agent for the range of indications described herein and/or a reagent for further synthesis methods and reactions.

### 2. Synthetic Methods

The compounds of the present invention can be synthesized using traditional solution synthesis techniques or solid phase chemistry methods. In either, the construction involves four phases: first, synthesis of the building blocks comprising recognition elements for the biological target receptor, plus one tether moiety, primarily for control and definition of conformation. These building blocks are assembled together, typically in a sequential fashion, in a second phase employing standard chemical transformations. The precursors from the assembly are then cyclized in the third stage to provide the macrocyclic structures. Finally, the post-cyclization processing fourth stage involving removal of protecting groups and optional purification provides the desired final compounds. Synthetic methods for this general type of macrocyclic structure are described in Intl. Pat. Appls. WO 01/25257, WO 2004/111077, WO 2005/012331, WO 2005/012332, WO 2008/033328 and WO 2008/130464, including purification procedures described in WO 2004/111077 and WO 2005/012331. See also U.S. Pat. Nos. 7,476,653 and 7,491,695.

In some embodiments of the present invention, the macrocyclic compounds may be synthesized using solid phase chemistry on a soluble or insoluble polymer matrix as previously defined. For solid phase chemistry, a preliminary stage involving the attachment of the first building block, also termed “loading,” to the resin must be performed. The resin utilized for the present invention preferentially has attached to it a linker moiety, L. These linkers are attached to an appropriate free chemical functionality, usually an alcohol or amine, although others are also possible, on the base resin through standard reaction methods known in the art, such as any of the large number of reaction conditions developed for the formation of ester or amide bonds. Some linker moieties for the present invention are designed to allow for simultaneous cleavage from the resin with formation of the macrocycle in a process generally termed “cyclization-release.” (van Maarseveen, J. H. Solid phase synthesis of heterocycles by cyclization/cleavage methodologies. Comb. Chem. High Throughput Screen. 1998, 1, 185-214; Ian W. James, Linkers for solid phase organic synthesis. Tetrahedron 1999, 55, 4855-4946; Eggenweiler, H.-M. Linkers for solid-phase synthesis of small molecules: coupling and cleavage techniques. Drug Discovery Today 1998, 3, 552-560; Backes, B. J.; Ellman, J. A. Solid support linker strategies. Curr. Opin. Chem. Biol. 1997, 1, 86-93. Of particular utility in this regard for compounds of the invention is the 3-thiopropionic acid linker. (Rojo, H.; Aimoto, S. Bull. Chem. Soc. Jpn. 1991, 64, 111-117; Zhang, L.; Tam, J. J. Am. Chem. Soc. 1999, 121, 3311-3320.)

Such a process provides material of higher purity as only cyclic products are released from the solid support and no contamination with the linear precursor occurs as would happen in solution phase. After sequential assembly of all the building blocks and tether into the linear precursor using known or standard reaction chemistry, base-mediated intramolecular attack on the carbonyl attached to this linker by an appropriate nucleophilic functionality that is part of the tether building block results in formation of the amide or ester bond that completes the cyclic structure as shown (Scheme 1). An analogous methodology adapted to solution phase can also be applied as would likely be preferable for larger scale applications.

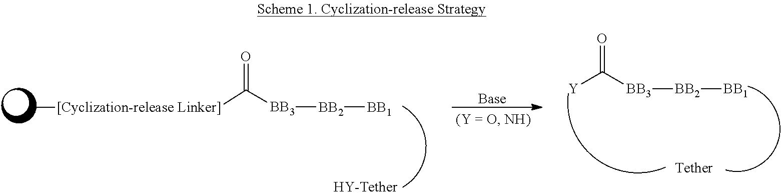

Although this description accurately represents the pathway for one of the methods of the present invention, the thioester strategy, another method of the present invention, that of ring-closing metathesis (RCM), proceeds through a modified route where the tether component is actually assembled during the cyclization step. However, in the RCM methodology as well, assembly of the building blocks proceeds sequentially, followed by cyclization (and release from the resin if solid phase). An additional post-cyclization processing step is required to remove particular byproducts of the RCM reaction, but the remaining subsequent processing is done in the same manner as for the thioester or analogous base-mediated cyclization strategy.

Moreover, it will be understood that steps including the methods provided herein may be performed independently or at least two steps may be combined. Additionally, steps including the methods provided herein, when performed independently or combined, may be performed at the same temperature or at different temperatures without departing from the teachings of the present invention.

Novel macrocyclic compounds of the present invention include those formed by a novel process including cyclization of a building block structure to form a macrocyclic compound comprising a tether component described herein. Accordingly, the present invention provides methods of manufacturing the compounds of the present invention comprising (a) assembling building block structures, (b) chemically transforming the building block structures, (c) cyclizing the building block structures including a tether component, (d) removing protecting groups from the building block structures, and (e) optionally purifying the product obtained from step (d). In some embodiments, assembly of the building block structures may be sequential. In further embodiments, the synthesis methods are carried out using traditional solution synthesis techniques or solid phase chemistry techniques.

A. General

Reagents and solvents were of reagent quality or better and were used as obtained from various commercial suppliers unless otherwise noted. DMF, DCM (CH₂Cl₂), DME, CH₃CN and THF used are of DriSolv® (EMD Chemicals, Inc., part of Merck KGaA, Darmstadt, Germany) or synthesis grade quality except for (i) deprotection, (ii) resin capping reactions and (iii) washing. NMP used for the amino acid (AA) coupling reactions is of analytical grade. DMF was adequately degassed by placing under vacuum for a minimum of 30 min prior to use. Homogeneous catalysts were obtained from Strem Chemicals, Inc. (Newbury Port, Mass., USA). Cbz-, Boc- and Fmoc-protected amino acids and side chain protected derivatives, including those of N-methyl and unnatural amino acids, were obtained from commercial suppliers or synthesized through standard methodologies known to those in the art. Ddz-amino acids were either synthesized by standard methods, or obtained commercially from Orpegen (Heidelberg, Germany) or Advanced ChemTech (Louisville, Ky., USA). Bts-amino acids were synthesized by established procedures. Hydroxy acids were obtained from commercial suppliers or synthesized from the corresponding amino acids as described in the literature (Tetrahedron 1989, 45, 1639-1646; Tetrahedron 1990, 46, 6623-6632; J. Org. Chem. 1992, 57, 6239-6256.; J. Am. Chem. Soc. 1999, 121, 6197-6205). Analytical TLC was performed on pre-coated plates of silica gel 60F254 (0.25 mm thickness) containing a fluorescent indicator and were visualized using the method(s) and reagent(s) indicated, for example using ultraviolet light (UV) and/or ceric-molybdic acid (CMA) solution (prepared by mixing 100 mL of sulfuric acid, 10 g ceric ammonium sulfate and 25 g ammonium molybdate).

The term “concentrated/evaporated/removed under reduced pressure” indicates removal of solvent and volatile components utilizing a rotary evaporator under either water aspirator pressure (typically 10-30 torr) or the stronger vacuum provided by a mechanical oil vacuum pump (“high vacuum,” typically ≦1 torr) as appropriate for the solvent being removed. Drying of a compound “in vacuo” or under “high vacuum” refers to drying using an oil vacuum pump at low pressure (≦1 torr). “Flash chromatography” was performed using silica gel 60 (230-400 mesh, EMD Chemicals, Still, W. C.; Kahn, M.; Mitra, A. J. Org. Chem. 1978, 43, 2923-2925) and is a procedure well-known to those in the art. “Dry pack” indicates chromatography on silica gel that has not been pre-treated with solvent, generally applied on larger scales for purifications where a large difference in R_{f} exists between the desired product and any impurities. For solid phase chemistry processes, “dried in the standard manner” is that the resin is dried first in air (1 h), and subsequently under vacuum (oil pump usually) until full dryness is attained (˜30 min to O/N). Glassware used in air and water sensitive reactions were dried in an oven at least O/N and cooled in a desiccator prior to use.

B. Amino acids

Amino acids, Boc- and Fmoc-protected amino acids and side chain protected derivatives, including those of N-methyl and unnatural amino acids, were obtained from commercial suppliers [for example Advanced ChemTech (Louisville, Ky., USA), Astatech (Bristol, Pa., USA), Bachem (Bubendorf, Switzerland), Chemlmpex (Wood Dale, Ill., USA), Novabiochem (subsidiary of Merck KGaA, Darmstadt, Germany), PepTech (Burlington, Mass., USA), Synthetech (Albany, Oreg., USA)] or synthesized through standard methodologies known to those in the art. Ddz-amino acids were either obtained commercially from Orpegen (Heidelberg, Germany) or Advanced ChemTech (Louisville, Ky., USA) or synthesized using standard methods utilizing Ddz-OPh or Ddz-N₃. (Birr, C.; Lochinger, W.; Stahnke, G.; Lang, P. Justus Liebigs Ann. Chem. 1972, 763, 162-172.) Bts-amino acids were synthesized by known methods. (Vedejs, E.; Lin, S.; Klapara, A.; Wang, J. J. Am. Chem. Soc. 1996, 118, 9796-9797. Also WO 01/25257, WO 2004/111077) In addition, N-alkyl amino acid derivatives were accessed via literature methods. (Hansen, D. W., Jr.; Pilipauskas, D. J. Org. Chem. 1985, 50, 945-950.)

C. Tethers

Tethers were obtained from the methods previously described in Intl. Pat. Appl. WO 01/25257, WO 2004/111077, WO 2005/012331, WO 2008/033328 and WO 2008/130464. See also U.S. Pat. Nos. 7,476,653 and 7,491,695. More tethers are described in U.S. Prov. Pat. Appl. 61/256,727. The preparation of additional tethers is provided in the Examples.

The following are specific tether intermediates utilized in the synthesis of compounds of the present invention, wherein PG indicates a nitrogen protecting group, such as, but not limited to, Boc, Fmoc, Ddz, Cbz or Alloc:

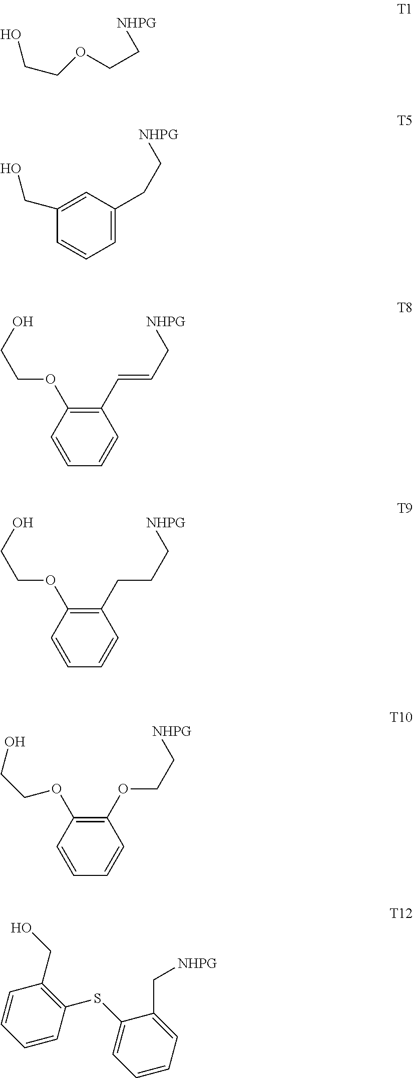

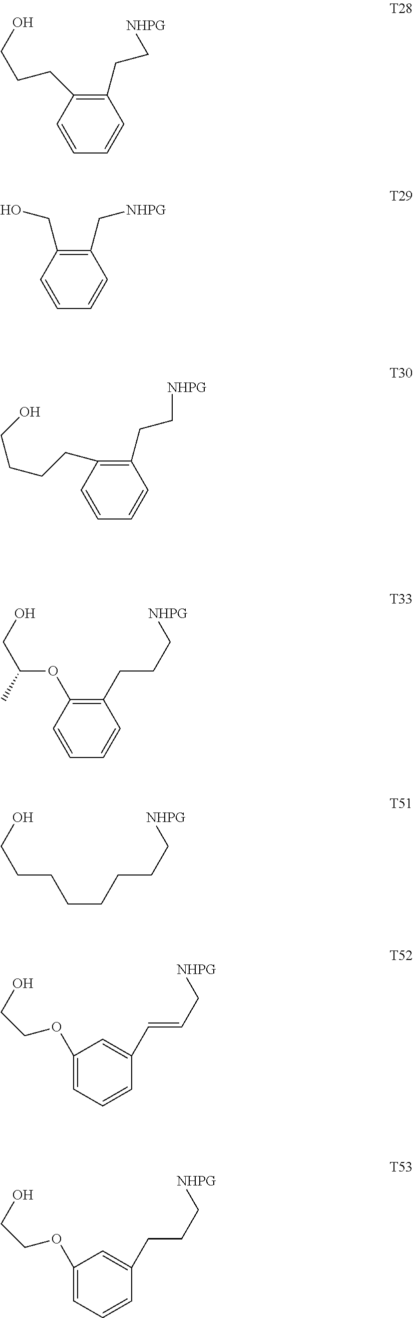

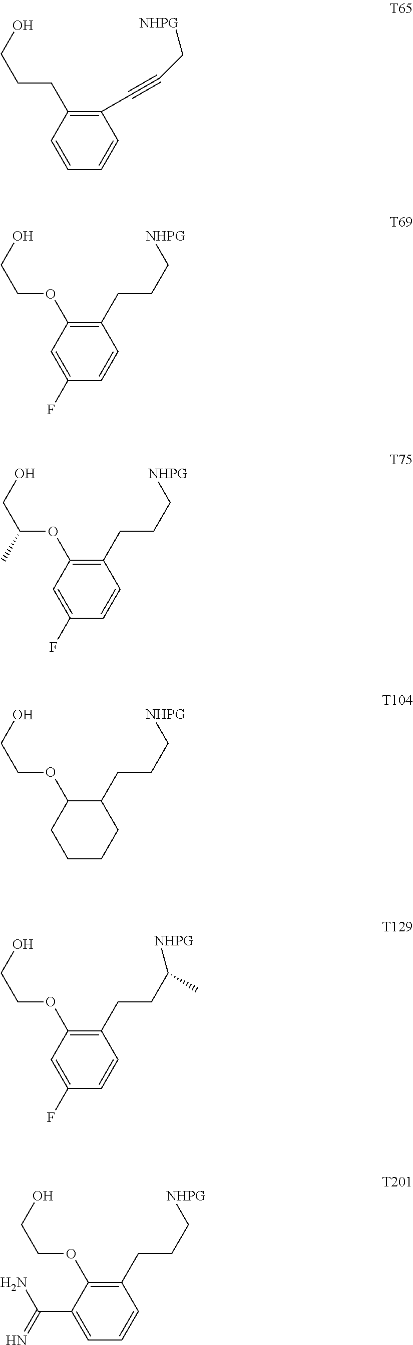

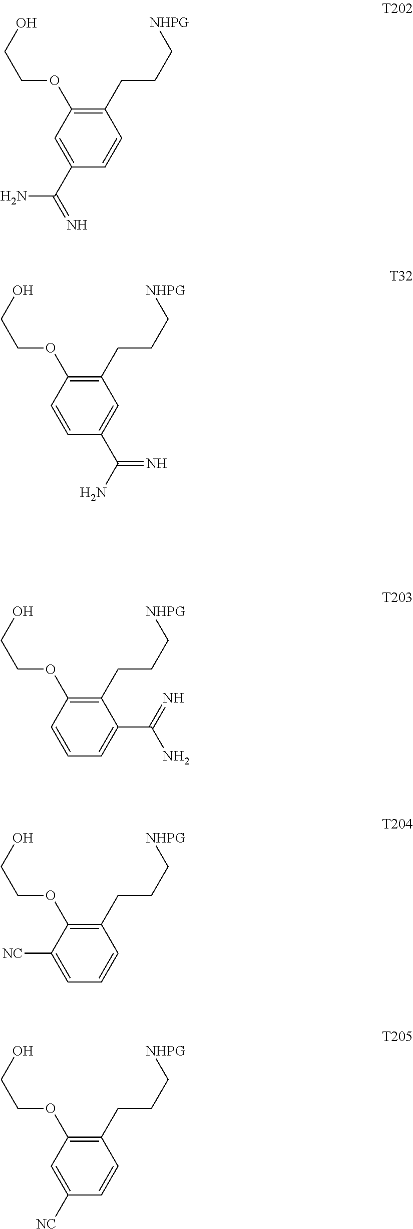

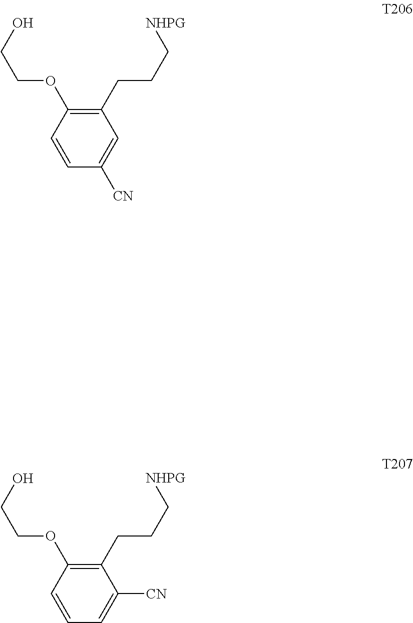

D. Solid and Solution Phase Techniques

Specific solid phase techniques for the synthesis of the macrocyclic compounds of the invention have been described in WO 01/25257, WO 2004/111077, WO 2005/012331, WO 2005/012332, WO 2008/033328, WO 2008/130464 and U.S. Prov. Pat. Appl. 61/256,727. Solution phase synthesis routes, including methods amenable to larger scale manufacture, were described in U.S. Patent Appl. Publ. Nos. 20061025566 and US 2007/0021331.

The table following provides information on the building blocks used for the synthesis of representative compounds of the present invention using the standard methods. These are directly applicable to solid phase synthesis. For solution phase syntheses, modified protection strategies from that illustrated are typically employed to permit the use of a convergent approach. Additional synthetic details for the solution phase construction of representative macrocyclic compounds of the invention are presented in the Examples.

For the syntheses in the table, the methodology outlined in Example 9B was employed. In the compounds with an amidine moiety on the tether, alternative strategies to that illustrated as described in Example 8H can also be used.

### Synthesis of Representative Compounds of the Invention

| Compound | AA₁ | AA₂ | AA₃ | TETHER |
|---|---|---|---|---|
| 451 | Fmoc-Cpg-OH | Fmoc-D-NMeAla-OH | Fmoc-D-Phe(4-CN)—OH | Boc-T69 |
| 452 | Fmoc-D-(3-Cl)Phe-OH | Fmoc-D-Val-OH | Fmoc-Cpa-OH | Ddz-T32(Boc) |
| 453 | Fmoc-D-Tyr(OMe)—OH | Fmoc-D-Val-OH | Fmoc-Nva-OH | Ddz-T32(Boc) |
| 454 | Fmoc-Cpg-OH | Fmoc-D-NMeAla-OH | Fmoc-Phe(4-CN)—OH | Boc-T69 |
| 455 | Fmoc-Cpg-OH | Fmoc-NMeAla-OH | Fmoc-D-Phe(4-CN)—OH | Boc-T69 |
| 456 | Fmoc-D-Cpg-OH | Fmoc-D-NMeAla-OH | Fmoc-D-Phe(4-CN)—OH | Boc-T69 |
| 457 | Fmoc-Cpg-OH | Fmoc-D-NMeAla-OH | Fmoc-D-Phe(4-CN)—OH | Boc-T129a |
| 458 | Fmoc-Cpg-OH | Fmoc-D-NMeAla-OH | Fmoc-D-Phe(4-CN)—OH | Boc-T75a |
| 459 | Fmoc-Cpg-OH | Fmoc-D-NMeAla-OH | Fmoc-D-Phe(4-CN)—OH | Boc-T33a |
| 460 | Fmoc-Cpg-OH | Fmoc-D-NMeAla-OH | Fmoc-D-Phe(4-CN)—OH | Ddz-T201(Boc) |
| 461 | Fmoc-Cpg-OH | Fmoc-D-NMeAla-OH | Fmoc-D-Phe(4-CN)—OH | Ddz-T202(Boc) |
| 462 | Fmoc-Cpg-OH | Fmoc-D-NMeAla-OH | Fmoc-D-Phe(4-CN)—OH | Ddz-T32(Boc) |
| 463 | Fmoc-Cpg-OH | Fmoc-D-NMeAla-OH | Fmoc-D-Phe(4-CN)—OH | Ddz-T203(Boc) |
| 464 | Fmoc-Cpg-OH | Fmoc-D-NMeAla-OH | Fmoc-D-Phe(4-CN)—OH | Boc-T9 |
| 465 | Fmoc-Cpg-OH | Fmoc-D-NMeAla-OH | Fmoc-D-Phe(4-CN)—OH | Boc-T8 |
| 466 | Fmoc-Cpg-OH | Fmoc-D-NMeAla-OH | Fmoc-D-Phe(4-CN)—OH | Boc-T65 |
| 467 | Fmoc-Ala-OH | Fmoc-D-NMeAla-OH | Fmoc-D-Phe(4-CN)—OH | Boc-T69 |
| 468 | Fmoc-Asp(OBut)—OH | Fmoc-D-NMeAla-OH | Fmoc-D-Phe(4-CN)—OH | Boc-T69 |
| 469 | Fmoc-Orn(Boc)—OH | Fmoc-D-NMeAla-OH | Fmoc-D-Phe(4-CN)—OH | Boc-T69 |
| 470 | Fmoc-Ser(But)-OH | Fmoc-D-NMeAla-OH | Fmoc-D-Phe(4-CN)—OH | Boc-T69 |
| 471 | Fmoc-Cpg-OH | Fmoc-D-NMeAla-OH | Fmoc-D-Phe(4-CN)—OH | Boc-T69 |
| 472 | Fmoc-Cpg-OH | Fmoc-D-NMeAla-OH | Fmoc-D-Phe(4-CN)—OH | Boc-T69 |
| 473 | Fmoc-Cpg-OH | Fmoc-D-NMeAla-OH | Fmoc-D-Phe(4-CN)—OH | Boc-T5 |
| 474 | Fmoc-Cpg-OH | Fmoc-D-NMeAla-OH | Fmoc-D-Phe(4-CN)—OH | Boc-T51 |
| 475 | Fmoc-Cpg-OH | Fmoc-D-NMeAla-OH | Fmoc-D-Phe(4-CN)—OH | Boc-T12 |
| 476 | Fmoc-Cpg-OH | Fmoc-D-NMeAla-OH | Fmoc-D-Phe(4-CN)—OH | Boc-T29 |
| 477 | Fmoc-Cpg-OH | Fmoc-D-NMeAla-OH | Fmoc-D-Phe(4-CN)—OH | Boc-T1 |
| 478 | Fmoc-Cpg-OH | Fmoc-D-NMeAla-OH | Fmoc-D-Phe(4-CN)—OH | Boc-T28 |
| 479 | Fmoc-Cpg-OH | Fmoc-D-NMeAla-OH | Fmoc-D-Phe(4-CN)—OH | Boc-T10 |
| 480 | Fmoc-Cpg-OH | Fmoc-D-NMeAla-OH | Fmoc-D-Phe(4-CN)—OH | Boc-T104 |
| 481 | Fmoc-Cpg-OH | Fmoc-D-NMeAla-OH | Fmoc-D-Phe(4-CN)—OH | Boc-T30 |
| 482 | Fmoc-Cpg-OH | Fmoc-D-NMeAla-OH | Fmoc-D-Phe(4-CN)—OH | Boc-T52 |
| 483 | Fmoc-Cpg-OH | Fmoc-D-NMeAla-OH | Fmoc-D-Phe(4-CN)—OH | Boc-T53 |
| 484 | Fmoc-Cpg-OH | Fmoc-D-NMeAla-OH | Fmoc-D-Phe(4-CN)—OH | Boc-T69 |
| 485 | Fmoc-Cpg-OH | Fmoc-D-NMeAla-OH | Fmoc-D-Lys(Boc)—OH | Boc-T69 |
| 486 | Fmoc-Cpg-OH | Fmoc-D-NMeAla-OH | Fmoc-D-hLys(Boc)—OH | Boc-T69 |
| 487 | Fmoc-Cpg-OH | Fmoc-D-NMeAla-OH | Fmoc-D-Orn(Boc)—OH | Boc-T69 |
| 488 | Fmoc-Cpg-OH | Fmoc-D-NMeAla-OH | Fmoc-D-Arg(Boc2)—OH | Boc-T69 |
| 489 | Fmoc-Cpg-OH | Fmoc-D-NMeAla-OH | Fmoc-D-hArg(Boc2)—OH | Boc-T69 |
| 490 | Fmoc-Cpg-OH | Fmoc-D-NMeAla-OH | Fmoc-D-Gln-OH | Boc-T69 |
| 491 | Fmoc-Cpg-OH | Fmoc-D-NMeAla-OH | Fmoc-D-Cit-OH | Boc-T69 |
| 492 | Fmoc-Cpg-OH | Fmoc-D-NMeAla-OH | Fmoc-D-hCit-OH | Boc-T69 |
| 493 | Fmoc-Cpg-OH | Fmoc-D-NMeAla-OH | Fmoc-D-His-OH | Boc-T69 |
| 494 | Fmoc-Cpg-OH | Fmoc-D-NMeAla-OH | Fmoc-D-3-Pal-OH | Boc-T69 |
| 495 | Fmoc-Cpg-OH | Fmoc-D-NMeAla-OH | Fmoc-D-4-Pal-OH | Boc-T69 |
| 496 | Fmoc-Cpg-OH | Fmoc-D-NMeAla-OH | Fmoc-D-4-ThzAla-OH | Boc-T69 |
| 497 | Fmoc-Cpg-OH | Fmoc-D-NMeAla-OH | Fmoc-D-Phe(4-CN)—OH | Boc-T9 |
| 498 | Fmoc-Cpg-OH | Fmoc-D-NMeAla-OH | Fmoc-D-Phe(4-CONH2)—OH | Boc-T33a |
| 499 | Fmoc-Cpg-OH | Fmoc-D-NMeAla-OH | Fmoc-D-Phe(4-CONH2)—OH | Ddz-T202(Boc) |

3. Analytical Methods

¹H and ¹³C NMR spectra were recorded on a Varian Mercury 300 MHz spectrometer (Varian, Inc., Palo Alto, Calif.) and are referenced internally with respect to the residual proton signals of the solvent unless otherwise noted. ¹H NMR data are presented, using the standard abbreviations, as follows: chemical shift (δ) in ppm (multiplicity, integration, coupling constant(s)). The following abbreviations are used for denoting signal multiplicity: s=singlet, d=doublet, t=triplet, q=quartet, quint=quintet, b or br=broad, and m=multiplet. Information about the conformation of the molecules in solution can be determined utilizing appropriate two-dimensional NMR techniques known to those skilled in the art. (Martin, G. E.; Zektzer, A. S. Two-Dimensional NMR Methods for Establishing Molecular Connectivity: A Chemist's Guide to Experiment Selection, Performance, and Interpretation, John Wiley & Sons: New York, 1988, ISBN 0471187070.)

HPLC analyses were performed on a Waters Alliance® system 2695 running at 1 mL/min using an Xterra® MS C18 column (or comparable) 4.6×50 mm (3.5 μm) and the indicated gradient method. A Waters 996 PDA provided UV data for purity assessment (Waters Corporation, Milford, Mass.). For certain analyses, an LCPackings (Dionex Corporation, Sunnyvale, Calif.) splitter (50:40:10) allowed the flow to be separated in three parts. The first part (50%) was diverted to a mass spectrometer (Micromass® Platform 11 MS equipped with an APCI probe) for identity confirmation. The second part (40%) went to an evaporative light scattering detector (ELSD, Polymer Laboratories, now part of Varian, Inc., Palo Alto, Calif., PLELS1000™) for purity assessment and the last portion (10%) went to a chemiluminescence nitrogen detector (CLND, Antek® Model 8060, Antek Instruments, Houston, Tex., part of Roper Industries, Inc., Duluth, Ga.) for quantitation and purity assessment. Each detector could also be used separately depending on the nature of the analysis required. Data was captured and processed utilizing the most recent version of the Waters Millennium® software package.

Representative standard HPLC conditions used for the analysis of compounds of the invention are presented below:

Typical Chromatographic Conditions

Column: XTerra RP18, 3.5 μm, 4.6×100 mm (or equivalent)

Detection (PDA): 220-320 nm

Column Temperature: 35±10° C.

Injection Volume: 10 μL

Flow Rate: 1 mL/min

Run Time: 20.0 min

Data Acquisition Time: 17.0 min

Mobile Phase A: Methanol (or Acetonitrile)

Mobile Phase B: Water

Mobile Phase C: 10% TFA in Water

Gradient A4

| Time (min) | % A | % B | % C |
|---|---|---|---|
| 0.00 | 5.0 | 85.0 | 10.0 |
| 5.00 | 65.0 | 25.0 | 10.0 |
| 9.00 | 65.0 | 25.0 | 10.0 |
| 14.00 | 90.0 | 0.0 | 10.0 |
| 17.00 | 90.0 | 0.0 | 10.0 |
| 17.50 | 5.0 | 85.0 | 10.0 |
| 20.00 | 5.0 | 85.0 | 10.0 |

Gradient B4

| Time (min) | % A | % B | % C |
|---|---|---|---|
| 0.00 | 5.0 | 85.0 | 10.0 |
| 6.00 | 50.0 | 40.0 | 10.0 |
| 9.00 | 50.0 | 40.0 | 10.0 |
| 14.00 | 90.0 | 0.0 | 10.0 |
| 17.00 | 90.0 | 0.0 | 10.0 |
| 17.50 | 5.0 | 85.0 | 10.0 |
| 20.00 | 5.0 | 85.0 | 10.0 |

The following table summarizes HPLC retention times for representative compounds of the invention.

TABLE
HPLC Retention Times for Representative Compounds of the Invention

| Compound | t_{R} (min) | Gradient |
|---|---|---|
| 454 | 6.15 | B4 |
| 455 | 6.32 | B4 |
| 456 | 6.27 | B4 |
| 457 | 7.05 | B4 |
| 458 | 6.87 | B4 |
| 459 | 6.36 | B4 |
| 461 | 4.69 | B4 |
| 464 | 6.00 | B4 |
| 465 | 5.99 | B4 |
| 466 | 6.13 | B4 |
| 467 | 5.99 | B4 |
| 471 | 6.15 | B4 |
| 473 | 4.61 | B4 |
| 475 | 6.91 | B4 |
| 476 | 6.20 | B4 |
| 477 | 6.17 | B4 |
| 478 | 6.36 | B4 |
| 479 | 5.20 | B4 |
| 480 | 6.86 | B4 |
| 481 | 5.39 | B4 |
| 482 | 5.64 | B4 |
| 484 | 7.17 | B4 |
| 485 | 5.45 | B4 |
| 487 | 4.91 | B4 |
| 488 | 5.66 | B4 |
| 490 | 5.93 | B4 |
| 491 | 5.93 | B4 |
| 492 | 6.27 | B4 |
| 493 | 5.46 | B4 |
| 494 | 5.48 | B4 |
| 495 | 5.48 | B4 |
| 496 | 6.68 | B4 |
| 498 | 7.01 | B4 |

Enantiomeric and diastereomeric purity were assessed using appropriate chiral HPLC columns using a Waters Breeze system (or comparable). Although other packing materials can be utilized, particularly useful columns for these analyses are: Chiralpalc AS-RH and Chiralcel OD-RH (Chiral Technologies, West Chester, Pa., USA).

Preparative HPLC purifications were performed on final deprotected macrocycles using the Waters FractionLynx® system, on an XTerra® MS C18 column (or comparable) 19×100 mm (5 μm). The injections were done using an At-Column-Dilution configuration with a Waters 2767 injector/collector and a Waters 515 pump running at 2 mL/min. The mass spectrometer, HPLC, and mass-directed fraction collection are controlled via MassLynx® software version 3.5 with FractionLynx®. Fractions (13×125 mm tubes) shown by MS analysis to contain the product were evaporated under reduced pressure, most typically on a centrifugal evaporator system (Genevac® HT-4 (Genevac Inc, Valley Cottage, N.Y.), ThermoSavant Discovery®, SpeedVac® or comparable (Thermo Electron Corporation, Waltham, Mass.) or, alternatively, lyophilized. Compounds were then thoroughly analyzed by LC-MS-UV-ELSD-CLND analysis for identity confirmation, purity and quantity assessment.

Automated medium pressure chromatographic purifications were performed on an Isco CombiFlash® 16x system with disposable silica or C18 cartridges that permitted up to sixteen (16) samples to be run simultaneously (Teledyne Isco, Inc., Lincoln, Nebr.). MS spectra were recorded on a Waters Micromass® Platform II or ZQ™ system. HRMS spectra were recorded with a VG Micromass ZAB-ZF spectrometer. Chemical and biological information were stored and analyzed utilizing the ActivityBase® database software (ID Business Solutions Ltd., Guildford, Surrey, UK).

The table below presents analytical data for representative compounds of the present invention.

TABLE
Analytical Data for Representative Compounds of the Invention

| Compound | Molecular | MW Calc | MS [(M + H)+] | |
|---|---|---|---|---|
| No. | Formula | (g/mol) | Found | Other MS Peaks |
| 451 | C30H39N6O4F | 566.7 | 567 | — |
| 452 | C32H43N6O4Cl | 611.2 | 611 | — |
| 453 | C32H46N6O5 | 594.7 | 595 | — |
| 454 | C30H39N6O4F | 566.7 | 567 | 550 (M − NH3) |
| 455 | C30H39N6O4F | 566.7 | 567 | 550 (M − NH3) |
| 456 | C30H39N6O4F | 566.7 | 567 | 550 (M − NH3) |
| 457 | C31H41N6O4F | 580.7 | 581 | 564 (M − NH3) |
| 458 | C31H41N6O4F | 580.7 | 581 | 564 (M − NH3) |
| 459 | C31H42N6O4 | 562.7 | 563 | 546 (M − NH3) |
| 460 | C31H42N8O4 | 590.7 | 591 | — |
| 461 | C31H42N8O4 | 590.7 | 591 | — |
| 462 | C31H42N8O4 | 590.7 | 591 | — |
| 463 | C31H42N8O4 | 590.7 | 591 | — |
| 464 | C30H40N6O4 | 548.7 | 549 | 532 (M − NH3) |
| 465 | C30H38N6O4 | 546.7 | 547 | 530 (M − NH3) |
| 466 | C30H36N6O4 | 544.6 | 545 | 528 (M − NH3) |
| 467 | C28H37N6O4F | 540.6 | 541 | 524 (M − NH3) |
| 468 | C29H37N6O6F | 584.6 | 585 | — |
| 469 | C30H42N7O4F | 583.7 | 584 | — |
| 470 | C28H37N6O5F | 556.6 | 557 | — |
| 471 | C30H39N6O4F | 566.7 | 567 | 550 (M − NH3) |
| 472 | C30H39N6O4F | 566.7 | 567 | — |
| 473 | C28H36N6O3 | 504.6 | 505 | 488 (M − NH3) |
| 474 | C27H42N6O3 | 498.7 | 499 | — |
| 475 | C33H38N6O3S | 598.8 | 599 | 582 (M − NH3) |
| 476 | C27H34N6O3 | 490.6 | 491 | 482 (M − NH3) |
| 477 | C23H34N6O4 | 458.6 | 459 | 482 (M − NH3) |
| 478 | C31H42N6O3 | 546.7 | 547 | 530 (M − NH3) |
| 479 | C29H38N6O5 | 550.6 | 551 | 534 (M − NH3) |
| 480 | C30H46N6O4 | 554.7 | 555 | 538 (M − NH3) |
| 481 | C29H38N6O4 | 534.7 | 535 | — |
| 482 | C30H38N6O4 | 546.7 | 547 | 530 (M − NH3) |
| 483 | C30H40N6O4 | 548.7 | 549 | — |
| 484 | C32H41N6O6F | 624.7 | 625 | — |
| 485 | C26H40N5O4F | 505.6 | 506 | — |
| 486 | C27H42N5O4F | 519.7 | 520 | — |
| 487 | C25H38N5O4F | 491.6 | 492 | — |
| 488 | C26H40N7O4F | 533.6 | 534 | — |
| 489 | C27H42N7O4F | 547.7 | 548 | — |
| 490 | C25H36N5O5F | 505.6 | 506 | — |
| 491 | C26H39N6O5F | 534.6 | 535 | 492 (M + H − CONH), |
| | | | | 450 |
| 492 | C27H41N6O5F | 548.7 | 549 | 506 (M + H − CONH), |
| | | | | 449 |
| 493 | C26H35N6O4F | 514.6 | 515 | — |
| 494 | C28H36N5O4F | 525.6 | 526 | — |
| 495 | C28H36N5O4F | 525.6 | 526 | — |
| 496 | C26H34N5O4FS | 531.6 | 532 | — |
| 497 | C30H37N5O4 | 531.6 | 532 | — |
| 498 | C31H41N5O5 | 563.7 | 564 | — |
| 499 | C31H41N7O5 | 591.7 | 592 | — |

Notes
1. Molecular formulas and molecular weights are calculated automatically from the structure via ActivityBase software (ID Business Solutions, Ltd., Guildford, Surrey, UK).
2. M + H obtained from LC-MS analysis using standard methods with gradient B4.
3. All analyses conducted on material after preparative purification.

### 3. Biological Methods

The compounds of the present invention can be evaluated for their ability to interact with serine protease enzymes. Such methods are well-established and known to those in the art. In addition, the activity of matriptase specifically can be investigated using time-domain near IR fluorescence (NIRF) imaging permitting in vitro and in vivo evaluation of inhibitory activity. (Napp, J.; Dullin, C.; Mueller, F.; et al. Int. J. Cancer 2010, 127, 1958-1974.) A similar method for imaging the activity of matriptase-1 in tumors involves using fluorescence microscopy and labeled antibodies. (Darragh, M. R.; Schneider, E. L.; Lou, J.; et al. Canc. Res. 2010, 70, 1505-1512.) Genetically altered mice lacking the St14 gene that encodes matriptase-1 provide an animal model for exploration of the effects of modulation of the enzyme. List, K.; Kosa, P.; Szabo, R.; Bey, A. L.; Wang, C. B.; Molinolo, A.; Bugge, T. H. Am. J. Pathol. 2009, 175, 1453-1463.)

### A. Inhibition Assay

Multiple literature methods for studying the level of inhibition of serine protease enzymes are available. As one example (Sisay, M. T.; Steinmetzer, T.; Stirnberg, M.; et al. J. Med. Chem. 2010, 53, 5523-5535), the activity of matriptase-1 or matriptase-2 in the conditioned medium of HEK-MT2 cells, of the purified catalytic domain of matriptase-2 and of recombinant matriptase (catalytic domain; Enzo Life Sciences, Lörrach, Germany) are assayed in Tris saline buffer (50 mM Tris, 150 mM NaCl, pH 8.0) at 37° C. by monitoring the release of para-nitroaniline from the chromogenic substrate Boc-Gln-Ala-Arg-para-nitroanilide (Bachem, Bubendorf, Switzerland) at 405 nm using a Cary 100 UV-vis spectrophotometer (Varian, Darmstadt, Germany). Kₘ values are determined with eight different substrate concentrations in duplicate experiments. Inhibition assays are performed in duplicate or triplicate measurements with three (for matriptase-2) or at least five (other experiments) different inhibitor concentrations. IC₅₀ values were obtained by nonlinear regression according to equation v=v₀/(1+[I]/IC₅₀). Then 10 mM inhibitor stock solutions of 1-4 and leupeptin (Calbiochem. Darmstadt, Germany) and a 100 mM stock solution of Boc-Gln-Ala-Arg-para-nitroanilide are prepared in DMSO, and a 1 mM stock solution of aprotinin (Carl Roth, Karlsruhe, Germany) in H₂O. The final concentration of the substrate is 400 μM and of DMSO was 1.5%. Into a cuvette containing 979 μL prewarmed assay buffer, 11 μL of a test sample solution and 4 μL of a substrate solution are added and thoroughly mixed. The reaction is initiated by adding 6 μL of an enzyme solution (5 μg/6 μL total protein of the conditioned medium of HEK-MT2 cells; 28 ng/6 μL purified catalytic domain of matriptase-2; 3 ng/6 μL of matriptase) and followed over 20 min.

Use of another method for determining inhibition of a representative serine protease, matriptase-1, by representative compounds of the present invention is shown in the Examples below.

### B. Pharmacokinetic Analysis of Representative Compounds of the Invention

The pharmacokinetic behavior of compounds of the invention can be ascertained by methods well known to those skilled in the art. (Wilkinson, G. R. “Pharmacokinetics: The Dynamics of Drug Absorption, Distribution, and Elimination” in Goodman & Giiman's The Pharmacological Basis of Therapeutics, Tenth Edition, Hardman, J. G.; Limbird, L. E., Eds., McGraw Hill, Columbus, Ohio, 2001, Chapter 1.) The following method was used to investigate the pharmacokinetic parameters (elimination half-life, total plasma clearance, etc.) for intravenous, subcutaneous and oral administration of compounds of the present invention. See also Intl. Pat. Publ. WO 2008/033328 and WO 2008/130464 and U.S. Pat. Nos. 7,476,653 and 7,491,695.

### C. Cancer and Metastasis Models

A vast array of different animal models are available to determine the in vivo efficacy of compounds of the invention for treatment of cancers of all types. These include, but are not limited to, mouse models (Cespedes, M. V.; Casanova, I.; Parreño, M.; Mangues, R. Clin. Transl. Oncol. 2006, 8, 318-329), human xenograft models (Kerbel, R. S. Cancer Biol. Ther. 2003, 2, 5134-S139), genetically engineered mouse models (Walrath, J. C.; Hawes, Van Dyke, T.; Reilly, K. M. Adv. Cancer Res. 2010, 106, 113-164) and metastatic rodent models (Eccles, S. A.; Box, G.; Court, W.; Sandie, J.; Dean, C. J. Cell. Biophys. 1994, 279-291; Hoffman, R. M. Invest. New Drugs 1999, 17, 343-359. Man, S.; Munoz, R.; Kerbel, R. S. Cancer Metastasis Rev. 2007, 26, 737-747). Some specific methods applicable to the compounds of the invention are presented in the Examples.

### D. Skin Disease Models

Animal models, in particular in rodent species, are available to study the effects of compounds of the present invention for the treatment of skin and tissue disorders. (Magin, T. M. Exp. Dermatol. 2004, 13, 659-660.) Genetically-modified mouse models of inflammatory skin diseases have been developed and provide other systems in which the efficacy of the compounds can be examined. (Haase, I.; Pasparakis, M.; Krieg, T. J. Dermatol. 2004, 31, 704-719.)

### E. Inflammatory Disease Models

To determine the utility of compounds of the invention in the treatment of inflammatory disorders, they can be studied in appropriate animal disease models. (Brodmerkel, C. M.; Vaddi, K. Curr. Opin. Biotechnol, 2003, 14, 652-658.) A host of such models are known, including for rheumatoid arthritis (Hegen, M.; Keith, J. C. Jr.; Collins, M.; Nickerson-Nutter, C. L. Ann. Rheum. Dis. 2008, 67, 1505-1515), osteoarthritis (Bendele, A. M. J. Musculoskelet. Neuronal. Interact. 2001, 1, 363-376; van den Berg, W. B. Curr. Rheumatol. Rep. 2008, 10, 26-29), inflammatory bowel diseases, such as Crohn's and ulcerative colitis (Wirtz, S.; Neurath, M. F. Int. J. Colorectal. Dis. 2000, 15, 144-160; Wirtz, S.; Neurath, M. F. Adv. Drug Deliv. Rev. 2007, 59, 1073-1083) and atherosclerosis (Russell, J. C.; Proctor, S. D. Cardiovasc. Pathol. 2006, 15, 318-330; Zadelaar, S.; Kleemann, R.; Verschuren, L.; et al. Arterioscler. Thromb. Vase. Biol. 2007, 27, 1706-1721).

### F. Respiratory Disease Models

A number of animal model systems are known that can be utilized to evaluate the efficacy of compounds of the invention in the treatment of COPD (Fox, J. C.; Fitzgerald, M. F. Curr. Opin. Pharmacol. 2009, 9, 231-242.), asthma (Nials, A. T.; Uddin, S. Dis. Model Mech. 2008, 1, 213-220), cystic fibrosis (Carvalho-Oliveira, I.; Scholte, B. J.; Penque, D. Expert Rev. Mol. Diagn. 2007, 7, 407-417), bronchitis (Nikula, K. J.; Green, F. H. Inhal. Toxicol. 2000, 12, 123-153), chronic respiratory infections (Kukavica-Ibrulj, I.; Levesque, R. C. Lab. Anim. 2008, 42, 389-412) and respiratory allergies (Pauluhn, J.; Mohr, U. Exp. Toxicol. Pathol. 2005, 56, 203-234).

Sheep models have proven to be effective for a number of respiratory disorders including asthma, COPD, allergic rhinitis and cystic fibrosis. (Abraham, W. M. Pulm. Pharmacol. Ther. 2008, 21, 743-754.)

### G. Iron Homeostasis Models

Animal models have been developed for iron transport disorders (Andrews, N. C. Adv. Exp. Med. Biol. 2002, 509, 1-17), as well as for the study of diseases involving iron metabolism (Latunde-Dada, G. O.; McKie, A. T.; Simpson, R. J. Biochim. Biophys. Acta 2006, 1762, 414-423).

### 4. Pharmaceutical Compositions

The macrocyclic compounds of the present invention or pharmacologically acceptable salts thereof according to the invention may be formulated into pharmaceutical compositions of various dosage forms. To prepare the pharmaceutical compositions of the invention, one or more compounds, including optical isomers, enantiomers, diastereomers, racemates or stereochemical mixtures thereof, or pharmaceutically acceptable salts thereof as the active ingredient is intimately mixed with appropriate carriers and additives according to techniques known to those skilled in the art of pharmaceutical formulations.

A pharmaceutically acceptable salt refers to a salt form of the compounds of the present invention in order to permit their use or formulation as pharmaceuticals and which retains the biological effectiveness of the free acids and bases of the specified compound and that is not biologically or otherwise undesirable. Examples of such salts are described in Handbook of Pharmaceutical Salts: Properties, Selection, and Use, Wermuth, C. G. and Stahl, P. H. (eds.), Wiley-Verlag Helvetica Acta, Zürich, 2002 [ISBN 3-906390-26-8]. Examples of such salts include alkali metal salts and addition salts of free acids and bases. Examples of pharmaceutically acceptable salts, without limitation, include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, xylenesulfonates, phenylacetates, phenylpropionates, phenyl butyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates.

If an inventive compound is a base, a desired salt may be prepared by any suitable method known to those skilled in the art, including treatment of the free base with an inorganic acid, such as, without limitation, hydrochloric acid, hydrobromic acid, hydroiodic, carbonic acid, sulfuric acid, nitric acid, phosphoric acid, and the like, or with an organic acid, including, without limitation, formic acid, acetic acid, propionic acid, maleic acid, succinic acid, mandelic acid, fumaric acid, malonic acid, pyruvic acid, oxalic acid, stearic acid, ascorbic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, 2-hydroxyethanesulfonic acid, benzenesulfonic acid, cyclohexyl-aminosulfonic acid or the like.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine, lysine and arginine; ammonia; primary, secondary, and tertiary amines such as ethylenediamine, N,N′-dibenzylethylenediamine, diethanolamine, choline, and procaine, and cyclic amines, such as piperidine, morpholine, and piperazine; as well as inorganic salts derived from sodium, calcium, potassium, magnesium, manganese, iron, copper, zinc, aluminum, and lithium.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation may also be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used.

Compositions for injection will include the active ingredient together with suitable carriers including propylene glycol-alcohol-water, isotonic water, sterile water for injection (USP), emulPhor™-alcohol-water, cremophor-EL™ or other suitable carriers known to those skilled in the art. These carriers may be used alone or in combination with other conventional solubilizing agents such as ethanol, propylene glycol, or other agents known to those skilled in the art.

Where the macrocyclic compounds of the present invention are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges and pastilles, wherein the active ingredient is formulated with a carrier such as sugar and acacia, tragacanth or gelatin and glycerin.

Compositions for rectal administration include suppositories containing a conventional suppository base such as cocoa butter.

Compositions suitable for transdermal administration include ointments, gels and patches.

Other compositions known to those skilled in the art can also be applied for percutaneous or subcutaneous administration, such as plasters.

Further, in preparing such pharmaceutical compositions comprising the active ingredient or ingredients in admixture with components necessary for the formulation of the compositions, other conventional pharmacologically acceptable additives may be incorporated, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, dextrose, lactose, glucose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

In some embodiments, the composition is provided in a unit dosage form such as a tablet or capsule.

In further embodiments, the present invention provides kits including one or more containers comprising pharmaceutical dosage units comprising an effective amount of one or more compounds of the present invention.

The present invention further provides prodrugs comprising the compounds described herein. The term “prodrug” is intended to mean a compound that is converted under physiological conditions or by solvolysis or metabolically to a specified compound that is pharmaceutically active. The “prodrug” can be a compound of the present invention that has been chemically derivatized such that, (i) it retains some, all or none of the bioactivity of its parent drug compound, and (ii) it is metabolized in a subject to yield the parent drug compound. The prodrug of the present invention may also be a “partial prodrug” in that the compound has been chemically derivatized such that, (i) it retains some, all or none of the bioactivity of its parent drug compound, and (ii) it is metabolized in a subject to yield a biologically active derivative of the compound. Known techniques for derivatizing compounds to provide prodrugs can be employed. Such methods may utilize formation of a hydrolyzable coupling to the compound.

The present invention further provides that the compounds of the present invention may be administered in combination with a therapeutic agent used to prevent and/or treat metabolic and/or endocrine disorders, gastrointestinal disorders, cardiovascular disorders, obesity and obesity-associated disorders, central nervous system disorders, bone disorders, genetic disorders, hyperproliferative disorders and inflammatory disorders. Exemplary agents include analgesics (including opioid analgesics), anesthetics, antifungals, antibiotics, antiinflamrnatories (including nonsteroidal anti-inflammatory agents), anthelmintics, antiemetics, antihistamines, antihypertensives, antipsychotics, antiarthritics, antitussives, antivirals, cardioactive drugs, cathartics, chemotherapeutic agents (such as DNA-interactive agents, antimetabolites, tubulin-interactive agents, hormonal agents, and agents such as asparaginase or hydroxyurea), corticoids (steroids), antidepressants, depressants, diuretics, hypnotics, minerals, nutritional supplements, parasympathomimetics, hormones (such as corticotrophin releasing hormone, adrenocorticotropin, growth hormone releasing hormone, growth hormone, thyrptropin-releasing hormone and thyroid stimulating hormone), sedatives, sulfonamides, stimulants, sympathomimetics, tranquilizers, vasoconstrictors, vasodilators, vitamins and xanthine derivatives.

Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses for veterinary purposes, and for drug screening and drug development purposes.

In therapeutic use for treatment of conditions in mammals (i.e. humans or animals) for which a modulator, such as an agonist, of the ghrelin receptor is effective, the compounds of the present invention or an appropriate pharmaceutical composition thereof may be administered in an effective amount. Since the activity of the compounds and the degree of the therapeutic effect vary, the actual dosage administered will be determined based upon generally recognized factors such as age, condition of the subject, route of delivery and body weight of the subject. The dosage can be from about 0.1 to about 100 mg/kg, administered orally 1-4 times per day. In addition, compounds can be administered by injection at approximately 0.01-20 mg/kg per dose, with administration 1-4 times per day. Treatment could continue for weeks, months or longer. Determination of optimal dosages for a particular situation is within the capabilities of those skilled in the art.

### 5. Methods of Use

The compounds of the present invention can be used for the prevention and treatment of a range of medical conditions including those described herein and further including, but not limited to, hyperproliferative disorders, inflammatory disorders, tissue disorders, cardiovascular disorders, respiratory disorders, viral infections and combinations thereof where the disorder may be the result of multiple underlying maladies. In particular embodiments, the disease or disorder is cancer.

According to a further aspect of the invention, there is provided a method for the treatment of hyperproliferative disorders such as tumors, cancers, and neoplastic disorders, as well as premalignant and non-neoplastic or non-malignant hyperproliferative disorders. In particular, tumors, cancers, and neoplastic tissue that can be treated by the present invention include, but are not limited to, malignant disorders such as breast cancers, osteosarcomas, angiosarcomas, fibrosarcomas and other sarcomas, leukemias, lymphomas, sinus tumors, ovarian, uretal, bladder, prostate and other genitourinary cancers, colon, esophageal and stomach cancers and other gastrointestinal cancers, lung cancers, myelomas, pancreatic cancers, liver cancers, kidney cancers, endocrine cancers, skin cancers and brain or central and peripheral nervous (CNS) system tumors, malignant or benign, including gliomas and neuroblastomas.

As used herein, “treatment” is not necessarily meant to imply cure or complete abolition of the disorder or symptoms associated therewith.

The compounds of the present invention can further be utilized for the preparation of a medicament for the treatment of a range of medical conditions including, but not limited to, hyperproliferative disorders, inflammatory disorders, respiratory disorders and viral infections.

Further embodiments of the present invention will now be described with reference to the following Examples. It should be appreciated that these Examples are for the purposes of illustrating embodiments of the present invention, and do not limit the scope of the invention.

### EXAMPLES

### Example 1

### Assay for Inhibition of a Representative Serine Protease

The following describes an assay for matriptase as a representative serine protease and is based upon reported methods. (Désilets, A.; Longpré, J.-M.; Beaulieu, M.-E.; Leduc, R. FEBS Lett. 2006, 580, 2227-2232.) Similar assays are applicable and available for other serine proteases.

Enzyme activities were monitored by measuring the release of fluorescence from AMC-coupled peptides (excitation, 360 nm; emission, 441 nm) in a FLX-800 TBE microplate reader (Bio-Tek Instruments, Winooski, Vt., USA). The purified human matriptase was active site titrated with the burst titrant 4-methylumbelliferyl-p-guanidino benzoate (MUGB). Enzymatic assays with matriptase were performed in Tris-HCl 100 mM containing 500 lg/mL BSA at pH 9. Human soluble furin was expressed, purified, titrated and assayed as described in the literature (Denault, J. B.; Lazure, C.; Day, R; Leduc, R. Protein Expr. Purif. 2000, 19, 113-124.) The purified HAT protein was active-site titrated with MUGB. Assays with HAT were performed in 50 mM Tris-HCl at pH 8.6.

Enzymes were diluted to concentrations ranging from 4 to 12.5 nM for furin, from 2 to 7 nM for matriptase and 20 pM for HAT and incubated with either 10 μM (for initial screening) at 37° C. or appropriate dilutions (for kinetic analysis), for example 0, 500, 1000, 2000 nM or 0, 250, 500, 1000, 2500, 5000 nM, of the test compound for 15 min at RT. Residual enzyme activity was measured by following the hydrolysis of a fluorogenic substrate (4 μM Boc-Arg-Val-Arg-Arg-AMC for furin, Boc-Gln-Ala-Arg-AMC for matriptase and 4 μM Boc-Val-Pro-Arg-AMC for HAT) (Bachem Bioscience, King of Prussia, Pa., USA). Saturation curves were performed in the presence of increasing concentrations of test compounds. Data from three independent experiments or more were typically averaged and residual velocities were plotted as a function of test compound concentration. Data were fitted by non-linear regression analysis to Equation (1) (Bieth, J. G. Methods Enzymol. 1995, 248, 59-84.) using the Enzfitter software (Biosoft, Ferguson, Mo., USA).

vᵢ/v₀=1−{([E]₀+[I]₀+Kᵢ₍ₐₚₚ₎)−(([E]₀+[I]₀+Kᵢ₍ₐₚₚ₎)²−4[E]₀[I]₀)^{1/2}}/2[E]₀ Equation (1):

where v₀ and vᵢ are the steady-state rates of substrate hydrolysis in the absence and presence of inhibitor, respectively, [E]₀, the initial concentration of enzyme, [I]₀, the initial concentration of inhibitor and Kᵢ₍ₐₚₚ₎ the substrate-dependent equilibrium dissociation constant. The substrate-independent constant Kᵢ was calculated using Equation (2) (Bieth, J. G. Methods Enzymol. 1995, 248, 59-84.),

Kᵢ=Kᵢ₍ₐₚₚ₎(1+[S]₀/Kₘ) Equation (2):

where [S]₀ is the initial concentration of substrate and Kₘ is the Michaelis-Menten constant for the enzyme-substrate interaction. To investigate the stability of the test compounds, 10 μM of the test compound was incubated at RT with a specific concentration of matriptase or HAT for a specific time. Proteins were then resolved by SDS-PAGE and revealed using the Gel Code blue stain reagent (Pierce Biotechnology, Rockford, Ill., USA).

The table presents results for matriptase inhibition for representative compounds of the invention.

TABLE
Inhibition of Matriptase by Representative Compounds of the Invention

| Compound | Velocity (FU/min) |
|---|---|
| 451 | 1590 |
| 454 | 2190 |
| 455 | 2440 |
| 456 | 2250 |
| 457 | 1750 |
| 458 | 812 |
| 459 | 140 |
| 461 | 812 |
| 464 | 1875 |
| 465 | 1125 |
| 467 | 2300 |
| 473 | 2375 |
| 475 | 2125 |
| 478 | 2440 |
| 479 | 2190 |
| 480 | 1875 |
| 481 | 1875 |
| 482 | 2500 |
| 485 | 2625 |
| 487 | 2500 |
| 488 | 2250 |
| 490 | 2440 |
| 491 | 2440 |
| 492 | 2440 |
| 493 | 2700 |
| 494 | 2125 |
| 495 | 2700 |
| 496 | 2700 |
| 498 | 580 |
| 499 | 937 |

Kᵢ's can be calculated from the velocity using nonlinear regression analysis. The model used is a competitive enzyme inhibition equation where K_{mObs}=Kₘ*(1+[I]/Kᵢ) and Y=Vₘₐₓ*X/(K_{mObs}+X). X is the substrate concentration. Y the velocity. (Equation 8.11, in Copeland, R. A. Enzymes, 2nd edition, Wiley, 2000. Kᵢ's were calculated using the GraphPad Prism 5 software (GraphPad Software, San Diego, Calif., USA). For example, compound 451 has a Kᵢ=1.46 μM and compound 459 has a Kᵢ=245 nM.

To determine selectivity of the inhibition, TTSPs and other serine proteases were incubated with test compound in the presence of the fluorogenic peptide Boc-Gln-Ala-Arg-AMC. Activity was measured for 20 min at 37° C.

### Example 2

### Protease Inhibition Assay

(Li, P.; Jiang, S.; Lee, S.-L.; et al. J. Med. Chem. 2007, 50, 5976-5983.) Bovine thrombin, Bowman-Birk inhibitor (BBI), and the fluorescent substrates were obtained commercially (Sigma Chemical Co., St. Louis, Mo.). Inhibitory activity of compounds of the invention to proteases was measured at room temperature in two different systems. In the first assay system, a reaction buffer of 100 mM Tris-HCl (pH 8.5) containing 100 mg/mL of bovine serum albumin was used. To a cuvette containing 170 μL of reaction buffer were added 10 μL of enzyme solution and 10 μL of inhibitor solution. After preincubation, a solution of the fluorescent peptide substrate (10 μL) was added and the cuvette content was mixed thoroughly. The residual enzyme activity was determined by following the change of fluorescence released by the hydrolysis of the substrates, using a fluorescent spectrophotometer (Hitachi F4500) with excitation wavelength of 360 nm and emission at 480 nm. For example, fluorescent peptide Boc-Gln-Ala-Arg-AMC was used as substrate for matriptase. Peptide Boc-Leu-Arg-Arg-AMC was used as substrate for thrombin. Hydrolysis rates were recorded in presence of six to seven different concentrations of the test compounds. The Kᵢ values were determined by Dixon plots from two sets of data with different concentrations of substrate.

The 70-kDa activated matriptase was isolated as described. (Lin, C.-Y.; Anders, J.; Johnson, M. D.; Dickson, R. B. J. Biol. Chem. 1997, 272, 27558-27564; Lin, C.-Y.; Anders, J.; Johnson, M.; Sang, Q. A.; Dickson, R. B. J. Biol. Chem. 1999, 274, 18231-18236.) The second assay system produced essentially identical results and made use of a Boc-Gln-Ala-Arg-AFC peptide as the substrate for matriptase in a buffer of 100 mM Tris (pH 8.3) containing 100 mg/mL of BSA. Assays were conducted with purified matriptase in a total volume of 200 μL in black wall 96-well plates using a Tecan Ultra fluorometer (Tecan, Durham, N.C.).

### Example 3

### Cell Culture Assay for Inhibition of a Representative Serine Protease

Test compounds were examined for their ability to inhibit matriptase activity in HEK293 cells transfected with matriptase cDNA. Test compounds were incubated for 18 h on mock and matriptase-transfected cells. Proteolytic activity in the media was measured using the fluorogenic peptide Boc-Gln-Ala-Arg-AMC.

### Example 4

### In Vitro Assay for Tumor Metastasis

(Galkin, A. V.; Mullen, L.; Fox, W. D.; Brown, J.; et al. Prostate 2004, 61, 228-235) CWR22RV1 cells are obtained from ATCC (Rockville, Md.) and cultured in RPMI-1640 medium supplemented with 7% fetal bovine serum (Omega Scientific, Tarzana, Calif.), 1% Penicillin-Streptomycin and 1% L-glutamine (Gibco, Grand Island, N.Y.). To study the effects of compounds of the invention on CWR22RV 1 cell proliferation rate, plated cells are divided into four groups and treated with test compound at 1, 10, or 25 mM concentrations or the vehicle solution on days 1, 3, and 5 after initial plating. Triplicate plates per group per day are used for the experiment. Cells are counted with a hemocytometer on days 3, 5, and 7. The Cell Invasion Assay (Chemicon, Temecula, Calif.) is used to evaluate the effect of compounds of the invention on CWR22RV1 cell invasion through a reconstituted basement membrane matrix of proteins (ECMatix; Chemicon). After rehydration of the ECMatix, CWR22RV1 cells (2×10⁵) in 0.4 mL of serum-free media with or without 25 mM test compound is added to the upper chambers and placed into lower chambers pre-filled with 0.75 mL of media containing 10% fetal bovine serum, also with or without 25 mM test compound and incubated at 378° C. for 48 h. At the end of the incubation, medium and any non-invading cells are removed and membranes stained with the supplied crystal violet solution. Membranes are then mounted onto glass slides and cells examined under a light microscope. Six membranes per group (±test compound treatment) are analyzed under 100× magnification. Five fields per membrane are randomly selected and the mean number of invading cells out of the total number of pores available counted. Percent of invading cells per observed field is calculated. The experiment is performed in duplicate.

### Example 5

### In vivo Assay for Tumor Metastasis

(Gallein, A. V.; Mullen, L.; Fox, W. D.; Brown, J.; et al. Prostate 2004, 61, 228-235.) Four- to six-week-old nude athymic BALB/c female mice (Charles Rivers Laboratories) are maintained in pathogen-free conditions. Mice are inoculated subcutaneously with minced tumor tissue together with reconstituted basement membrane (Matrigel; Collaborative Research, Bedford, Mass.) from the established androgen independent (AI) three CWR22R and CWRSA6 xenograft cell lines. After 4-10 days, mice with established tumors of approximately 5×5 mm³ receive either a test compound (50 or 25 mg/kg 2×/day 7×/wk i.p.) in saline or the vehicle alone at the same dosing schedule. Tumors are measured twice weekly with vernier calipers; and tumor volumes calculated by the formula (π/6)×(larger diameter)×(smaller diameter)² (Press, M. F.; Bernstein, L.; Thomas, P. A.; Meisner, L. F.; Zhou, J. Y.; Ma, Y.; Flung, G.; Robinson, R. A.; Harris, C.; El-Naggar, A.; Slamon, D. J.; Phillips, R. N.; Ross, J. S.; Wolman, S. R.; Flom, K. J. J. Clin. Oncol. 1997, 15, 2894-2904.) Animals are sacrificed 18-25 d post tumor inoculation and tumor tissue is snap frozen for analysis.

### Example 6

### In vivo Assay for Tumor Metastasis

Six week old Keratin-5-matriptase transgenic and littermate control mice (List, K.; Szabo, R.; Molinolo, A.; Sriuranpong, V.; Redeye, V.; Murdock, T.; Burke, B.; Nielsen, B. S.; Gutkind, J. S.; Bugge, T. H. Genes Dev. 2005, 19, 1934-1950.) are treated with one or more concentrations of the test compounds. The effect of the test compounds on the rate of proliferation of epidermal keratinocytes in the mid-lumbar region is then determined by comparison with the results from treatment with vehicle control.

### Example 7

### Chick Embryo Chorioallantoic Membrane Model

A literature method can be used to measure the ability of compounds of the invention to inhibit angiogenesis. (Ghiso, J. A. A.; et al. J. Cell. Biol. 1999, 147, 89-104.)

### Example 8

### Synthesis of Tethers

### A. Standard Procedure for the Synthesis of Tether T5

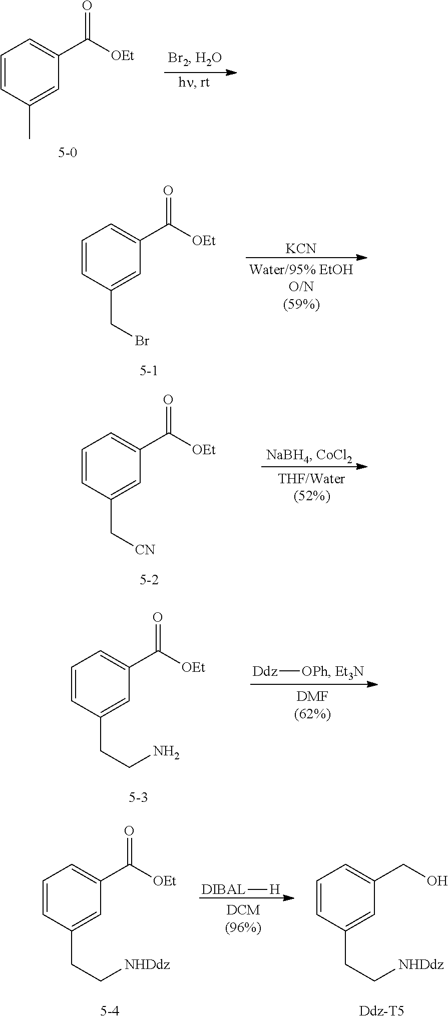

Step 5-1. To a solution of ethyl 3-methylbenzoate (5-0, 300 g, 1.83 mol, 1 eq) in distilled water (5 L) was added bromine (292.5 g, 1.83 mol) in one portion. This mixture was irradiated with two 200 W lamps. The lamps were placed outside the middle of the flask and a box was placed around the flask. The solution was stirred vigorously during the irradiation. The temperature rose to 45° C. and the solution turned from orange to yellow to almost colorless during the reaction. After 4 h (essentially a colorless solution), the lamps were turned off and the mixture allowed to cool to rt. The mixture was diluted with 2 L of DCM, then the aqueous phase extracted with 500 mL of DCM. The combined organic phases were washed with brine, then with a 10% sodium thiosulfate solution and finally brine (pH=5) again. The organic phase was dried over MgSO₄, filtered and the filtrate concentrated under reduced pressure to give 5-1 as a liquid, 96% yield, of sufficient quality to be used in the next step.

TLC (15% EtOAc/Hex): R_{f}: 0.58, detection: UV

Step 5-2. To a mixture of 5-1 (149 g, 0.611 mol) in ethanol (95%, 1 L) stirred at rt was added a solution of potassium cyanide (68 g, 1.7 eq) in distilled water (300 mL) dropwise using an addition funnel. (CAUTION: POISON! Potassium cyanide is a known poison and should be handled with adequate protection in a well-ventilated fumehood. Run the reaction in the presence of an HCN detector. All glassware has to be washed with water and acetone after the reaction and the washing solutions must be correctly disposed of in a container clearly identified CYANIDE! DANGER!) The solution became yellow during the addition. After the addition was completed, the reaction mixture was heated to 60° C. for 2 h, then stirred at rt overnight (reaction monitoring by TLC: 10% EtOAc/90% Hex; detection: UV, CMA). The solution was diluted with water (900 mL), then extracted with Et₂O (3×900 mL). The combined organic phases were washed twice with brine (2×), dried over MgSO₄, filtered and the filtrate evaporated under reduced pressure to afford an orange oil. The oil residue was purified by dry pack on silica gel with EtOAc/Hex (gradient, 5/95 to 15/85) to give 5-2 as a yellow solid (66 g, 59% for two steps).

TLC (30/70 EtOAc/Hex): R_{f}: 0.45, detection: UV);

¹H NMR: δ 1.6 ppm (2H, triplet), 3.8 ppm (3H, s), 4.4 ppm (2H, quartet), 7.4 to 7.6 ppm (2H, m), 8.0 to 8.1 ppm (2H, m).

Step 5-3. To a solution of 5-2 (220 g, 1.17 mol) in THF/water (4.6 L/2.3 L) at rt were added cobalt chloride (54.7 g, 0.23 mol), followed by sodium borohydride portionwise (132 g, 3.5 mol). Hydrogen evolution is observed. After the addition, the reaction was stirred O/N at rt. The mixture was filtered on Celite® and washed with 1 L THF. The THF was removed by evaporation under reduced pressure, then a solution of sodium hydroxide (0.5 N, 2 L) added and the mixture extracted with Et₂O (3×). The combined organic phases were washed with brine (2×), dried over Na₂SO₄, filtered and the filtrate concentrated under reduced pressure to give a crude liquid, 52% from 5-2, of adequate quality to be used directly in the next step.

TLC (50/50 EtOAc/Hex): R_{f}: baseline, detection: UV, ninhydrin.

Step 5-4. A solution of 5-3 (118 g, 0.61 mol), Ddz-OPh (213 g, 0.67 mol) and triethylamine (85 mL, 0.61 mol) in degassed DMF (200 mL) was stirred at 50° C. under a nitrogen atmosphere for 2 d. The mixture was then diluted in 2.5 L of water. The aqueous phase was extracted with Et₂O (3×). The combined organic phases were washed successively with water, sodium hydroxide (0.5 N, 2×) and brine (2×), dried over MgSO₄, filtered and the filtrate concentrated under reduced pressure to give a brown oil. The crude material was purified by dry pack (gradient, 15% EtOAc/Hex, 0.5% Et₃N to 25% EtOAc/Hex, 0.5% Et₃N; detection: UV+CMA) to give 156 g (62%) of 5-4.

TLC (25/75 EtOAc/Hex): R_{f}: 0.23, detection: UV+CMA.

Other protecting groups compatible with the reduction of Step 5-5, also can be employed at this stage and are attached using standard reaction conditions.
Step 5-5. To a solution of 5-4 (291.5 g, 0.7 mol) in DCM (2.1 L) at −78° C. was added diisobutyl aluminum hydride (DIBAL-H, 1.0 M in DCM, 2.1 L, 2.1 mol) through an addition funnel. Once the addition was complete, the solution was stirred at −78° C. for 2 h or until complete as indicated by TLC monitoring (50% EtOAc/Hex; detection: UV, ninhydrin). The reaction mixture was then quenched by dropping it slowly into a solution of tartaric acid (1.0 M, 4 L). The resulting mixture was extracted with DCM (3×). The combined organic phases were washed sequentially with a 0.6 M solution of EDTA tetrasodium salt (1×2 L) and brine (1×2 L), dried over MgSO₄, filtered and the filtrate concentrated under reduced pressure to give the product, Ddz-T5, as a yellow oil (251.4 g, 96%).

TLC (50/50, EtOAc/Hex): R_{f}: 0.25, detection: UV, ninhydrin;

¹H NMR: 8 (1.7 ppm (s, 6H, 2×CH₃), 2.8 ppm (t, 2H, 2×CH₂), 3.4 ppm (quartet, 2H, 2×CH₂), 3.8 ppm (s, 6H, 2×OCH₃), 4.7 ppm (s, 2H, CH₂), 6.3-6.5 ppm (m, 3H, 3×CH), 7.0-7.4 ppm (m, 4H, 4×CH).

### B. Standard Procedure for the Synthesis of Tether T-28

Also see U.S. Pat. No. 7,521,420.

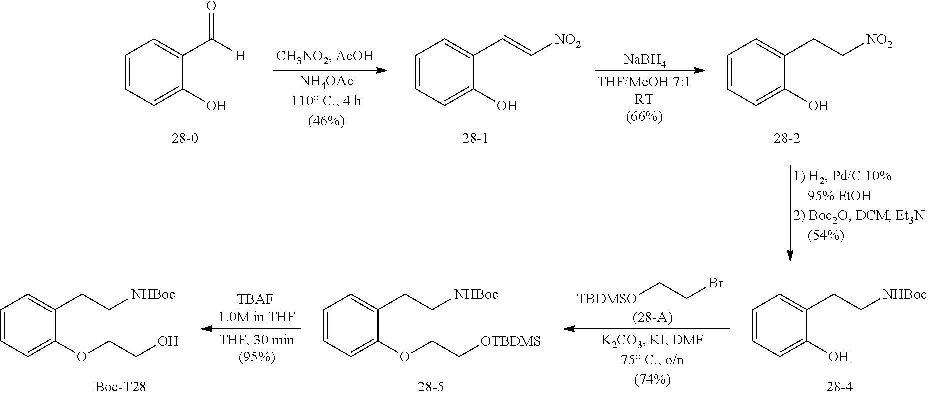

Step 28-1. (Tius, M. A. J. Am. Chem. Soc. 1992, 114, 5959.) To a solution of salicylaldehyde (28-0, 23.4 g, 0.19 mol, 1.0 eq) in acetic acid (115 mL) was added ammonium acetate (17 g, 0.22 mol, 1.15 eq) and nitromethane (39.5 mL, 0.73 mol, 3.8 eq). The mixture was heated at 110° C. for 4.5 h, then cooled at RT. The solvent was removed in vacuo, diluted in DCM, washed with brine (3×), dried over MgSO₄, filtered and the solvent evaporated under reduced pressure. The residue is purified by flash chromatography (gradient, 10%, then 20%, then 25% EtOAc/Hex) to yield 14.5 g (45.8%) of 28-1.

TLC (25/75 EtOAc/Hex): R_{f}=0.21, detection UV, CMA;

¹H NMR (CDCl₃): δ 8.16-8.11 (d, 1H), 7.98-7.93 (d, 1H), 7.44 (d, 1H), 7.43-7.32 (m, 1H), 7.32-6.98 (t, 1H), 6.87 (d, 1H).

Step 28-2. To a solution of 28-1 (14.5 g, 0.088 mol, 1.0 eq) in THF/MeOH (7/1, 500 mL) at 0° C., was added sodium borohydride (10.0 g, 0.26.0 mol, 3.0 eq) portion-wise. The reaction was warned at RT and monitored by TLC until completion. The reaction was quenched by a slow addition of water. The pH was adjusted with 1M HCl at pH 7-8. The THF was removed in vacuo, then the remaining mixture extracted with ether (3×). The organic phase was washed with brine (1×), dried over MgSU₄, filtered and the solvent evaporated under reduced pressure to give 9.6 g (66%) of 28-2 of sufficient purity to use in the next step.

TLC (25/75 EtOAc/Hex): R_{f}=0.23, detection: UV, CMA.

Step 28-3. To a solution of 28-2 (9.6 g, 0.058 mol, 1.0 eq) in EtOH 95% (200 mL) was added 10% Pd/C and hydrogen gas was bubbled in overnight. The mixture was filtered through Celite® and the solvent was evaporated under reduced pressure. The product was co-evaporated with EtOAc. The residue (7.9 g), 28-3, was used for the next step without any further purification.

TLC (25/75 EtOAc/Hex): R_{f}=0.0, detection: UV, CMA.

Step 28-4. To a solution of 28-3 (7.9 g, 0.058 mol, 1.0 eq) and Et₃N (16.2 mL, 0.12 mol, 2.0 eq) in DCM at 0° C. was added a solution of Boc₂O (12.7 g, 0.058 mol, 1.0 eq) in DCM dropwise. The reaction mixture was stirred overnight. The reaction mixture was washed with citrate buffer (2×) and brine (2×), dried over MgSO₄, filtered and the solvent evaporated under reduced pressure. The crude residue was purified by flash chromatography. (gradient, 20%, then 25% EtOAc/Hex) to provide 28-4 (7.4 g, 54%, 2 steps).

TLC (25/75 EtOAc/Hex): R_{f}=0.36, detection: UV, CMA.

Step 28-5. To a solution of 2-bromoethanol (2.29 g, 42.3 mmol, 1.0 eq) in THF (200 mL) was added imidazole (7.2 g, 105.8 mmol, 2.5 eq) then TBDMSC1 (6.7 g, 44.4 mmol, 1.05 eq). The reaction mixture was stirred 4 h; a white precipitate began forming after 2-5 min. Ether (200 mL) was added and the organic phase washed sequentially with a saturated solution of ammonium chloride (2×), a saturated solution of sodium bicarbonate (1×) and brine (1×), dried over MgSO₄, filtered and the solvent evaporated under reduced pressure. The product (28-A, 8.7 g, 86%) thus obtained was used directly for the next reaction.

TLC (25/75 EtOAc/Hex): R_{f}=0.80, detection: UV, CMA.

To a solution of 28-4 (4.2 g, 17.8 mmol, 1.0 eq), 28-A (6.4 g, 26.7 mmol, 1.5 eq) and potassium iodide (591 mg, 3.6 mmol, 0.2 eq) in DMF (40 mL) were added potassium carbonate (2.7 g, 19.6 mmol, 1.1 eq) and the mixture heated overnight at 75° C. After that period, TLC indicated the reaction was not completed, so 1 eq more of 28-A and potassium carbonate were added and the mixture stirred one extra night The DMF was removed under vacuum (oil pump). The oil residue was diluted in water and the product extracted with ether (3×). The organic phase was washed with brine (2×), dried over MgSO₄, filtered and the solvent evaporated under reduced pressure. The product was purified by flash chromatography (15% EtOAc/Hex) to yield 5.2 g (74%) of 28-5.

TLC (35/65 EtOAc/Hex): R_{f}=0.79, detection: UV, ninhydrin

¹H NMR (CDCl₃): δ 7.05 (m, 2H), 6.78 (m, 2H), 4.6 (bs, 1H), 3.95 (m, 2H), 3.88 (m, 2H), 3.28 (bq, 2H), 2.72 (t, 2H), 1.3 (s, 9H), 0.8 (s, 9H), 0.0 (s, 6H)

Step 28-6. To a solution of 28.5 (2.5 g, 13.3 mmol, 1.0 eq) in THF (20 mL) was added 1.0 M TBAF in THF (15.9 mL, 15.9 mmol, 1.2 eq) and the reaction stirred 30 min at room temperature. The reaction mixture was diluted with ether (150 mL), then washed with a saturated solution of ammonium chloride (2×) and brine (1×), dried over MgSO₄, filtered and the solvent evaporated under reduced pressure. The product was purified by flash chromatography (gradient, 25% to 40% EtOAc/Hex) to provide 3.5 g (94.6%) of Boc-T28.

TLC (5/65 EtOAc/Hex): R_{f}=0.21, detection: UV, ninhydrin;

¹H NMR (CDCl₃): δ 7.3 (td, 1H), 7.1 (dd, 1H), 6.86 (m, 2H), 4.9 (bs, 1H), 4.1 (m, 2H), 4.0 (m, 2H), 3.3 (bs, 2H), 2.8 (t, 2H), 1.4 (m, 9H);

¹³C NMR (CDCl₃): δ 157.2, 156.6, 130.8, 128.0, 127.6, 120.9, 111.4, 79.7, 69.8, 61.4, 40.9, 32.6, 28.6;

LC-MS (Gradient A4): t_{R}: 10.2 min; (M+H)⁺ 281, (M+H+Na)⁺ 304

### C. Standard Procedure for the Synthesis of Tether T29

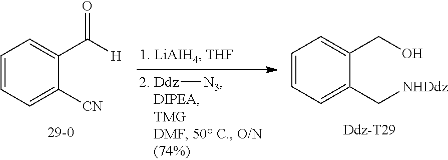

Step 29-1: To a solution of lithium aluminum hydride (LAH, 3 mol eq) in THF (DriSolv grade) at 0° C. was added, portion by portion, 3-cyanobenzaldehyde (29-0, 1 eq). The mixture was stirred at 0° C. for 1 h (or until the starting material disappeared), then heated at reflux (70° C.) in an oil bath under a nitrogen atmosphere O/N. To quench the reaction, the solution was cooled to 0° C. under nitrogen and the following added sequentially: water, NaOH (15%), then water (the ratio of 5 mL:5 mL:15 mL should be used for each 5 g of LAH). (CAUTION: hydrogen gas evolution). The solution was filtered, the salts washed with THF, and the combined filtrates concentrated under reduced pressure to give the crude amino alcohol, typically of sufficient purity to be used in the next step. (R_{f}: baseline, 30/70, EtOAc/Hex; detection: UV, ninhydrin).
Step 29-2: To a solution of the product from Step 29-1 (1 eq) and Ddz-N₃ (1.05 eq) in degassed DMF under a nitrogen atmosphere at 0° C. was added tetramethylguanidine (TMG, 1.05 eq). After 10 min, DIPEA (1.05 eq) was added, then the mixture stirred in an oil bath at 50° C. O/N. The mixture was concentrated under reduced pressure (oil pump) to remove DMF, then the residue dissolved in DCM, washed successively with citrate buffer (2×), saturated sodium bicarbonate (1×), and brine (2×), then dried over MgSO₄, filtered and the filtrate concentrated under reduced pressure. The crude material thus obtained was purified by flash chromatography (gradient, 50% EtOAc/Hex, 0.5% Et₃N to 60% EtOAc/Hex, 0.5M % Et₃N; DCM was added in the mixture to dissolve the residue at the beginning) to give the desired compound, Ddz-T29 (TLC: 50% Hex/EtOAc; detection: UV, ninhydrin).
Other typical nitrogen protecting groups, such as Fmoc, Boc, Cbz, can also be installed in Step 29-2 using standard reaction conditions. As an alternative, the reduction in Step 29-1 can be performed using sodium borohydride with cobalt chloride, followed by selective protection of the primary amine with Boc (as shown) or other suitable N-protecting group.

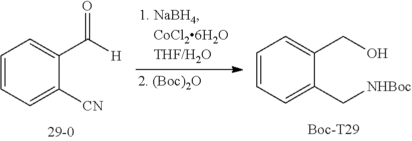

### D. Standard Procedure for the Synthesis of Tether T-30

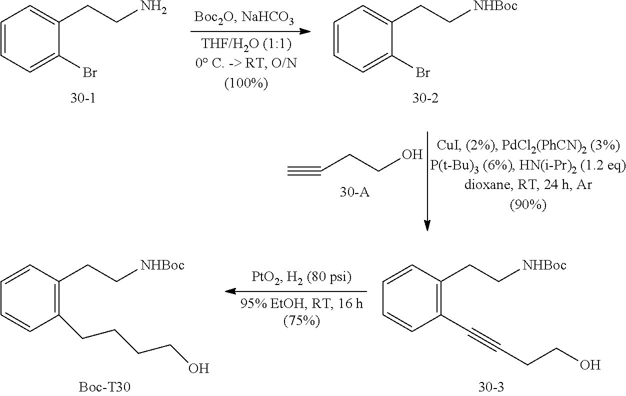

Step 30-1. To a solution of 2-bromophenethylamine (30-0, 5.0 g, 25.0 mmol, 1.0 eq) in 125 mL THF/H₂O (1:1) was added sodium bicarbonate (2.3 g, 27.5 mmol, 1.1 eq). The mixture was then cooled to 0° C. and Boc-anhydride (5.5 g 25.0 mmol, 1.0 eq) added in one portion. The mixture was stirred at 0° C. for 1 h, then allowed to warm to room temperature and stirred overnight. The solvent was evaporated under reduced pressure and the residue dissolved in EtOAc/H₂O (1:1). The separated organic phase was washed with H₂O (2×), saturated sodium chloride (2×), dried over magnesium sulfate, filtered and the filtrate evaporated under reduced pressure. The resulting yellow oil was diluted in DCM and evaporated under reduced pressure (procedure repeated 3×) to give 7.5 g (100%) of 30-1 as a white solid.

TLC (Hex/EtOAc, 7:3): R_{f}=0.75, detection: UV, ninhydrin

Step 30-2. To a flame dried flask under argon atmosphere was added 30-1 (6.3 g, 21.0 mmol, 1.0 eq), recrystallized copper (I) iodide (80.0 mg, 0.42 mmol, 0.02 eq, see procedure in Organometallics in Synthesis, 2^{nd} edition, Manfred Schlosser, Ed., 2002, p 669) and dichlorobis(benzonitrile) palladium (II) (242 mg, 0.63 mmol, 0.03 eq.). The flask was purged with argon (5-10 min) and 20 mL of anhydrous 1,4-dioxane were added followed by tri-tert-butylphosphine (10% (w/w) solution in hexanes, 385 uL, 1.26 mmol, 0.06 eq) and diisopropylamine (3.6 mL, 25.2 mmol, 1.2 eq). The mixture was then purged again with argon (5-10 min) and 3-butynol (30-A, 2.4 mL, 31.5 mmol, 1.5 eq) was added dropwise to the mixture and stirred 24 h at room temperature under argon with TLC monitoring. The mixture was diluted with EtOAc, filtered through a silica gel pad, and washed with EtOAc until there was no additional material eluting as indicated by TLC. The filtrate was evaporated under reduced pressure and the residue purified by flash chromatography (Hex:EtOAc, 7:3) to give 5.5 g (90%) of 30-2 as pale-yellow oil.

TLC (Hex/EtOAc, 7:3): R_{f}=0.20, detection: UV, CMA

Step 30-3. To a solution of Boc-amino alcohol 30-2 (6.1 g, 21.1 mmol, 1.0 eq) in 95% EtOH under nitrogen was added platinum (IV) oxide (445 mg, 2.11 mmol, 0.1 eq). The mixture was stirred 16 h at 80 psi H₂. (The reaction has also been successfully conducted at 1 atm H₂, RT, 24-36 h). The reaction was monitored by ¹H NMR by removal of a small 1.5 aliquot. When the reaction was complete, nitrogen was bubbled through the mixture for 10 min to remove excess hydrogen. The solvent was evaporated under reduced pressure, diluted with EtOAc, filtered through a silica gel pad, and washed with EtOAc until there was no additional material eluting as indicated by TLC. The filtrate was evaporated under reduced pressure and the residue purified by flash chromatography (Hex:EtOAc, 7:3) to give 4.5 g (75%) of Boc-T30 as a pale yellow oil.

¹H NMR (CDCl₁): δ 7.18-7.11, (m, 4H), 4.65, (bs, 1H), 3.72-3.65, (t, 2H), 3.32

(bs, 2H), 2.85-2.80, (t, 2H), 2.70-2.65, (t, 2H), 1.71-1.66 (m, 4H), 1.44 (s, 9H). Other N-protecting groups compatible with the reaction sequence of Steps 30-2 and 30-3 can also be employed.

### E. Standard Procedure for the Synthesis of Tether T-32

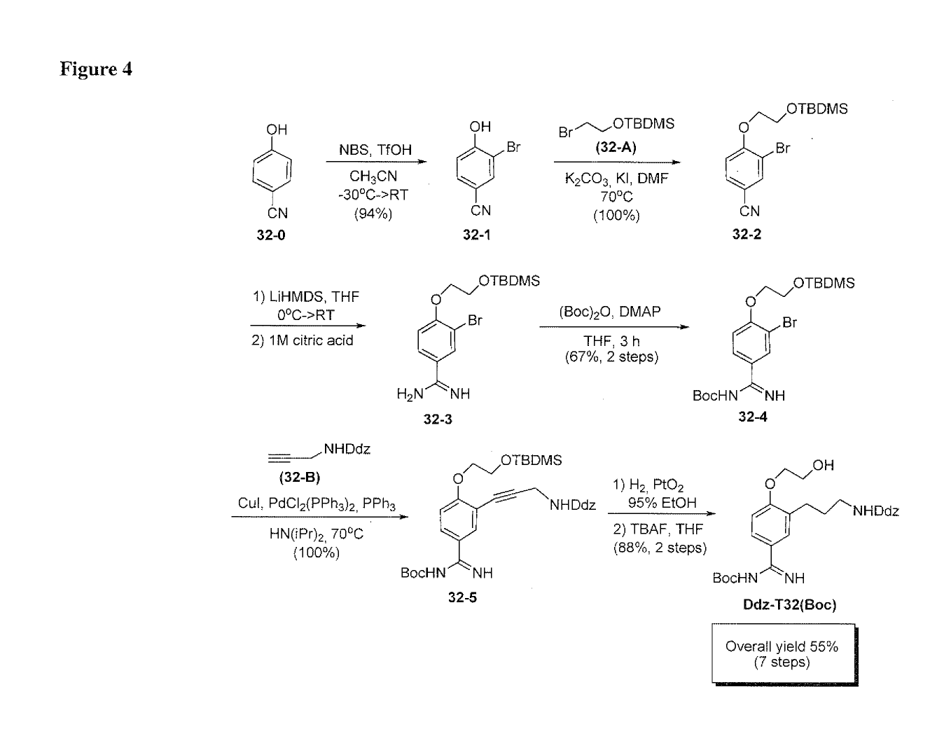

The reaction scheme for T32 is presented in FIG. 4.
Step 32-1. To a solution of 4-hydroxybenzonitrile (32-0, 15.0 g, 109 mmol, 1.0 eq) in CH₃CN (500 mL) at −30° C. was added triflic acid (11.6 mL, 131 mmol, 1.2 eq). NBS (20.3 g, 117 mmol, 1.05 eq) was added portion-wise such that the temperature did not rise above −10° C. A suspension was obtained and the solution became homogeneous after a few minutes. The reaction mixture was maintained at room temperature and stirred overnight. The solution was treated with aqueous saturated NaHCO₃ and the aqueous phase extracted with EtOAc (1×). The aqueous phase was acidified with 6M HCl and extracted with EtOAc. The organic phase was then extracted with aqueous saturated NH₄Cl (2×). The organic phase was dried over MgSO4, filtered and the filtrate concentrated under reduced pressure. If the final compound was found to contain too much succinimide (more then 10% by ¹H NMR) side product, the solid residue was stirred in water overnight, the precipitate filtered and dried overnight under vacuum (oil pump). ¹H NMR verified the identity of the desired compound, 32-1. The product was suitable to be used for the next step without further purification (yield: 94%).

TLC (80% EtOAc, 20% hexanes): R_{f}=0.47; detection: UV and KMnO₄.

Step 32-2. To a solution of 32-1 (11.3 g, 57.1 mmol, 1.0 eq) in DMF (300 mL) were added potassium carbonate (8.7 g, 62.8 mmol, 1.1 eq), potassium iodide (1.9 g, 11.4 mmol, 0.2 eq) and TBDMS-bromoethanol (32-A, 20.5 g, 85.7 mmol, 1.5 eq). The resulting mixture was stirred at 70° C. overnight. The mixture was cooled to room temperature, brine added and the layers separated. The aqueous phase was extracted with ether and the combined organic phases were extracted with brine (2×). The organic phase was dried over MgSO₄ and concentrated under reduced pressure. The residue was purified by flash chromatography (20% EtOAc, 80% hexanes) to give 32-2 as a yellow solid (yield: 100%).

TLC (40% EtOAc, 60% hexanes): R_{f}=0.63; detection: UV and KMnO₄.

Step 32-3. To a solution of 32-2 (548 mg, 1.5 mmol, 1 eq) in THF (10 mL) at 0° C. was added lithium hexamethyldisilazide (LHMDS, 1M in THF, 3.0 mL, 3.0 mmol, 2.0 eq), then the mixture stirred at room temperature for 2 h. Citric acid (1M, 5 mL) was added and the mixture stirred for 1 h. Ether was added, the layers separated, then the organic phase extracted with 1M citric acid (2×). The combined aqueous phases were adjusted with 3M NaOH to pH=14, then extracted with CH₂Cl₂ (4×). The organic phases was dried over K₂CO₃ and concentrated under reduced pressure. The 32-2 thus obtained was used directly for the next step.

TLC (20% EtOAc, 80% hexanes): Rₜ=baseline; detection: UV and KMnO₄.

Step 32-4. To a solution of 32-3 (1.5 mmol. 1.0 eq) in THF (6 mL) were added (Boc)₂O (371 mg, 1.7 mmol, 1.1 eq) and DMAP (18 mg, 0.15 mmol, 0.1 eq) and the mixture stirred for 3 h. Brine was added and the aqueous phase extracted with ether (3×). The combined organic phase was dried over MgSO₄ and concentrated under reduced pressure. The residue was purified by flash chromatography (30% EtOAc, 70% hexanes) to give a white solid, 32-4 (yield: 67%, 2 steps). Aqueous sodium hydroxide (1N) in dioxane can also be used as a base in this step with comparable yield.

TLC (30% EtOAc, 70% hexanes): R_{f}=0.37; detection: UV and KMnO₄.

Step 32-5. To a solution of 32-4 (8.2 g, 17.3 mmol, 1.0 eq) in diisopropylamine (100 mL) was added Ddz-propargylamine (32-B, 9.6 g, 34.6 mmol, 2.0 eq) and the mixture degassed with Ar for 20-30 min. PPh₃ (546 mg, 2.08 mmol, 0.12 eq), PdCl₂(PPh₃)₂ (730 mg, 1.04 mmol, 0.06 eq) and CuI (131 mg, 0.69 mmol, 0.04 eq) were added and the resulting mixture stirred at 70° C. overnight. The solution was filtered through a silica gel pad and rinsed with EtOAc, then the solvent evaporated under reduced pressure. The resulting residue was purified by flash chromatography (40% EtOAc, 60% hexane) to give 32-5 as an orange solid (yield: 100%).

TLC (40% EtOAc, 60% hexanes): R_{f}=0.27; detection: UV and CMA.

Step 32-6. To a solution of 32-5 (15.0 g, 22.2 mmol, 1.0 eq) in 95% ethanol (100 mL) was added PtO₂ (500 mg, 2.2 mmol, 0.1 eq) and hydrogen gas was bubbled through the solution for 1 h. The resulting mixture was stirred at room temperature overnight. If the reaction was not finished at that time (¹H NMR), 0.1 eq. PtO₂ more was added, hydrogen gas bubbled through the solution and the mixture stirred overnight again. Ar was bubbled through the reaction to eliminate the excess hydrogen and the solution filtered through a silica gel pad and the pad rinsed with EtOAc. The combined solvent was evaporated under reduced pressure. The 32-6 obtained was used for the next step (yield: 100%).
Step 32-7. To a solution of 32-6 (14.5 g, 21.5 mmol. 1.0 eq) in THF (100 mL) was added 1M TBAF in THF (32.3 mL, 32.3 mmol, 1.5 eq) and the mixture stirred for 1 h. Brine was added and the aqueous phase extracted with EtOAc. The combined organic phases were dried over MgSO₄, filtered and the filtrate concentrated under reduced pressure. The residue was purified by flash chromatography (100% EtOAc) to give Ddz-T32(Boc) (yield: 88%).

TLC (100% EtOAc): R_{f}=0.24; detection: UV and CMA.

¹H NMR (CDCl₃): δ 7.74 (1H, dd), 7.35 (1H, d), 6.72 (1H, d), 6.53-6.49 (2H, m), 3.61-3.29 (1H, m), 5.06 (1H, t), 4.25-4.01 (2H, m), 3.91-3.89 (2H, m), 3.73 (3H, s), 2.99 (2H, dd), 2.63 (2H, t), 1.71 (8H, broad s), 1.53 (9H, s);

¹³C NMR (CDCl₃, ppm): δ 163.8, 162.2, 161.0, 159.7, 155.9, 149.4, 130.0, 129.1, 128.0, 126.8, 110.8, 98.1, 80.9, 79.3, 69.7, 61.3, 55.5, 39.1, 29.3, 28.5, 26.7.

### F. Standard Procedure for the Synthesis of Tether T52 and Tether T53

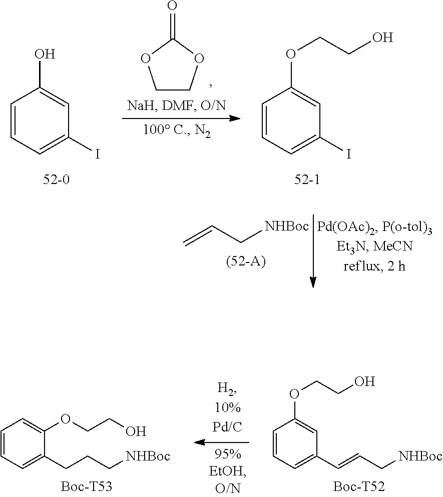

Step T52-1. To a solution of 3-iodophenol (52-0, 1.0 eq) in DMF (DriSolv®) is added sodium hydride (60% in mineral oil, 0.1 eq) portion-wise (CAUTION! Hydrogen gas is seen to evolve). The reaction is heated for 1 h at 100° C. under nitrogen, then ethylene carbonate is added and the reaction mixture heated O/N at 100° C. The reaction is monitored by TLC (conditions: 25/75 EtOAc/Hex). The reaction mixture is allowed to cool, then the solvent evaporated under reduced pressure. The residual oil is diluted in Et₂O (1.5 L), then washed sequentially with 1 N sodium hydroxide (3×) and brine (2×), dried with MgSO₄, filtered and the filtrate evaporated under reduced pressure. The crude product is distilled under vacuum or purified by flash chromatography to provide 52-1.
Step T52-2. To a solution of 52-1 (1.0 eq) and Boc-allyl amine (1.3 eq) in CH₃CN is bubbled argon for 20-30 min. Freshly distilled Et₃N (refluxed for 4 h on CaH₂ then distilled, 3.6 eq) is added and argon bubbled for 10-15 min. Tris(o-tolyl)phosphine (0.03 eq) and Pd(OAc)₂ (0.03 eq) are then added. The reaction is stirred at reflux atmosphere for 2 h with TLC monitoring. If the reaction is not complete, longer time can be used. The volatiles are removed under reduced pressure and the residue purified by flash column chromatography to afford Boc-T52.
Step T52-3. To Boc-T52 (1.0 eq) is added 10% Pd/C (15% by weight) and 95% EtOH. The mixture was placed in a hydrogenation apparatus (Parr for example) under a pressure of hydrogen gas for 24 h. Monitoring can be performed by LC-MS or ¹H NMR. The mixture is filtered through a Celite® pad, then concentrated under reduced pressure to afford of Boc-T53, which can be purified by flash chromatography.

### G. Standard Procedure for Tethers T201

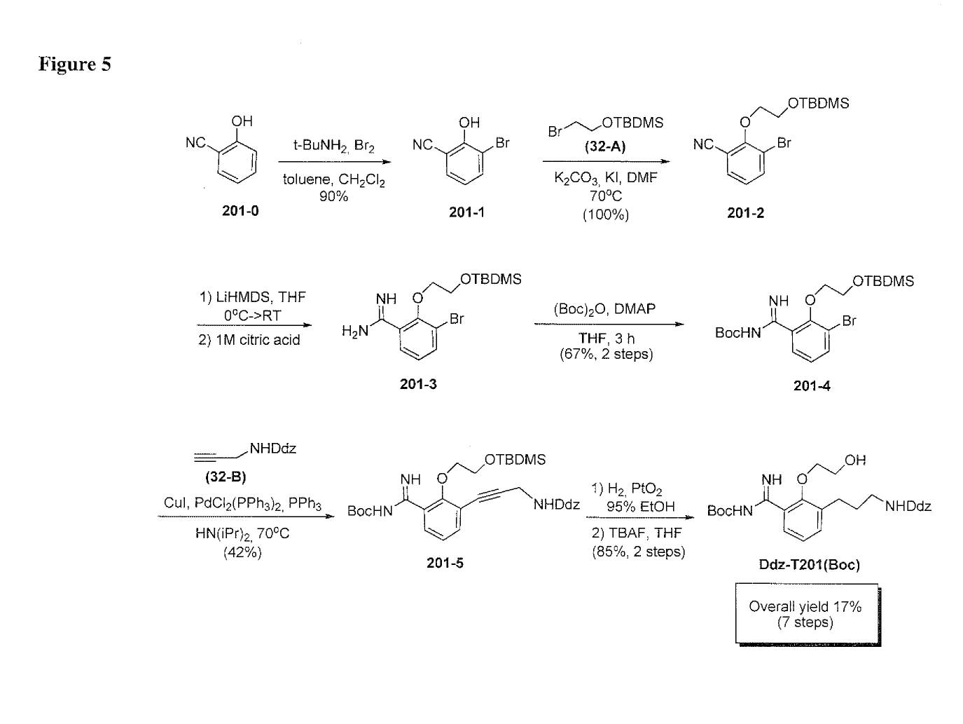

The reaction scheme for T201 is presented in FIG. 5.
Step 201-1. To a solution of t-butylamine (40 mL, 378 mmol, 3.0 eq) in toluene (320 mL) at −30° C. was slowly added Br₂ (7.1 mL, 139 mmol, 1.1 eq) (10 min). The mixture was cooled to −78° C. and 2-hydroxybenzonitrile (201-0, 15.0 g, 126 mmol, 1.0 eq) added in CH₂Cl₂ (80 mL). The 2-hydroxybenzonitrile was not very soluble in DCM and was added to the reaction as a suspension with a pipette. The heterogeneous mixture was cooled down slowly at room temperature and stirred overnight. Brine was added, the layers separated and the aqueous phase extracted with ethyl acetate. The organic phases were combined and extracted with 10% NaOH (2×). The aqueous phase was acidified with 6N HCl and extracted with CH₂Cl₂. The organic phase was dried over MgSO₄ and concentrated under reduced pressure to give 201-1 (yield: 90%).

TLC (60% EtOAc, 40% hexanes): R_{c}=0.32; detection: UV and KMnO₄.

Step 201-2. The conversion of 201-1 to 201-2 by alkylation with TBDMS-bromoethanol (32-A) was conducted essentially as described for the synthesis of 32-2 in Step 32-2.
Step 201-3. The formation of the amidine 201-3 from 201-2 was performed essentially as described for the synthesis of 201-3 in Step 32-3, except that 3 eq of LHMDS was used for the transformation and the reaction duration was 2-3 d.
Step 201-4. The protection of the amidine group of 201-3 with Boc was executed essentially as described for the synthesis of 32-4 in Step 32-4.
Step 201-5. The Sonogashira coupling reaction of 201-4 and Ddz-propargylamine (32-B) to give 201-5 was conducted essentially as described for the synthesis of 32-5 in Step 32-5. However, the coupling reaction was not complete and the starting material was treated a second time under the same conditions to provide the product.
Step 201-6. The hydrogenation and deprotection of 201-5 was performed essentially as described for the synthesis of Ddz-T32(Boc) in Step 32-6 to provide Ddz-T201(Boc).

¹H NMR (CDCl₃): δ 7.87 (1H, d), 7.28-7.25 (1H, m), 7.10 (1H, t), 6.51-6.46 (2H, m), 6.31 (1H, t), 5.30-5.20 (1H, m), 3.90-3.85 (2H, m), 3.85-3.80 (2H, m), 3.74 (6H, s), 3.15-3.05 (2H, m), 2.67 (2H, t), 1.85-1.71 (2H, m), 1.71 (6H, s), 1.53 (9H, s);

¹³C NMR (CDCl₃): δ 160.8, 155.6, 155.5, 135.6, 133.9, 129.9, 127.9, 125.0, 103.3, 98.2, 80.8, 79.8, 61.9, 60.6, 55.5, 40.2, 31.3, 29.5, 28.5, 27.1, 14.4.

### H. Standard Procedure for Tethers T202 and T203

These tethers can be prepared either by incorporating the amidine moiety into the tether prior to attachment to the remainder of the molecule as already described for tethers T32 and T201 or by using a nitrile as a masked amidine group, then converting the nitrite to the amidine. For the former approach, T202 can be accessed starting from 2-bromo-5-cyanophenol, while T203 can be accessed starting from 2-bromo-3-cyanophenol.

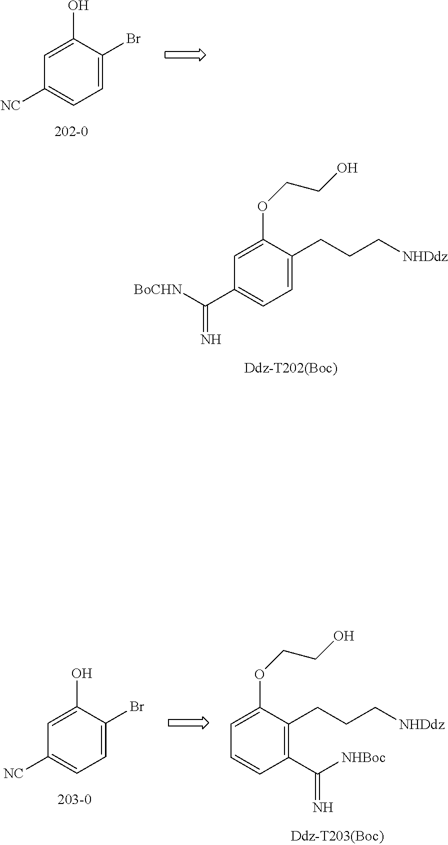

For the latter, the transformations as described for compound 451 can be employed on an appropriate macrocyclic nitrile as illustrated below.

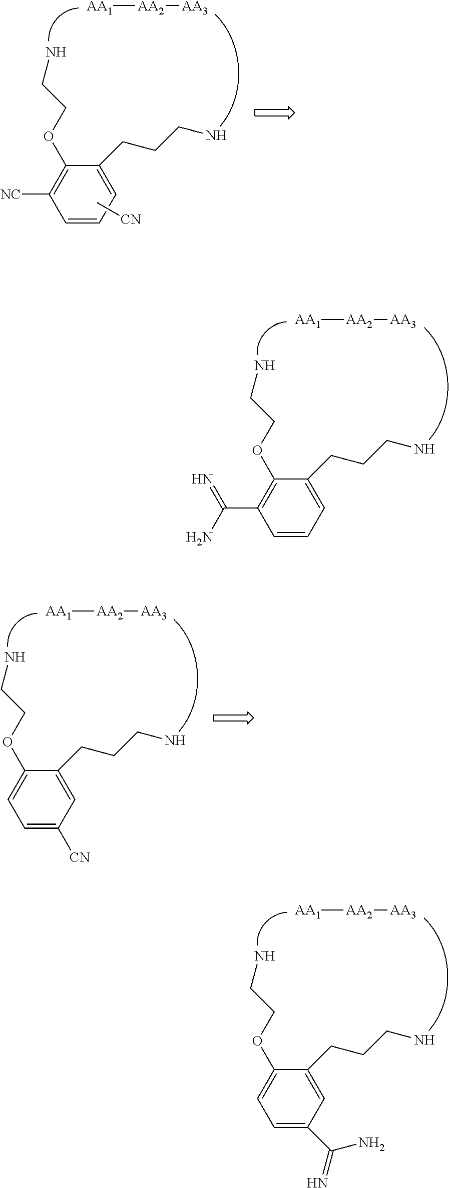

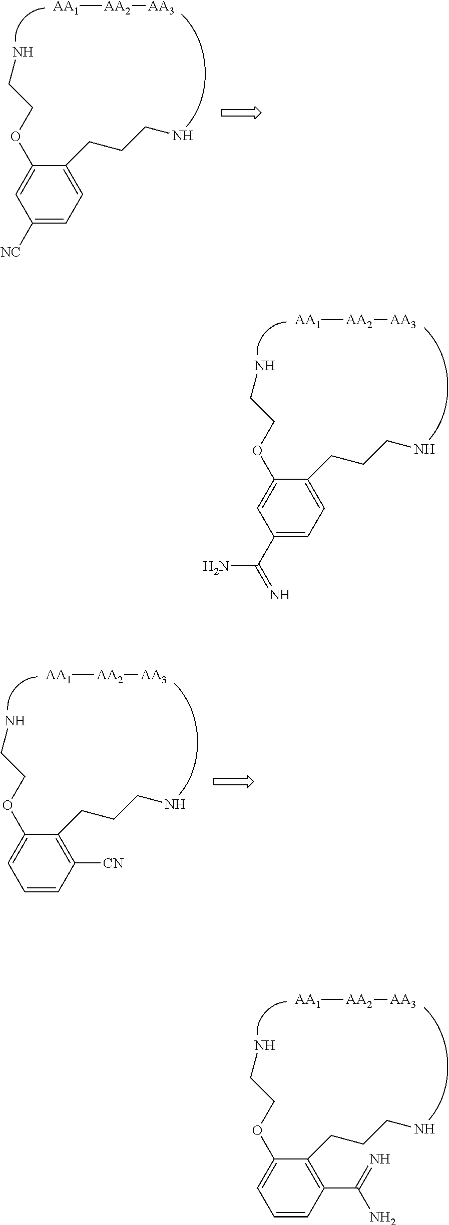

### Example 9

### Synthesis of Macrocycles

### A. Standard Procedure for the Synthesis of a Representative Macrocycle of the Invention

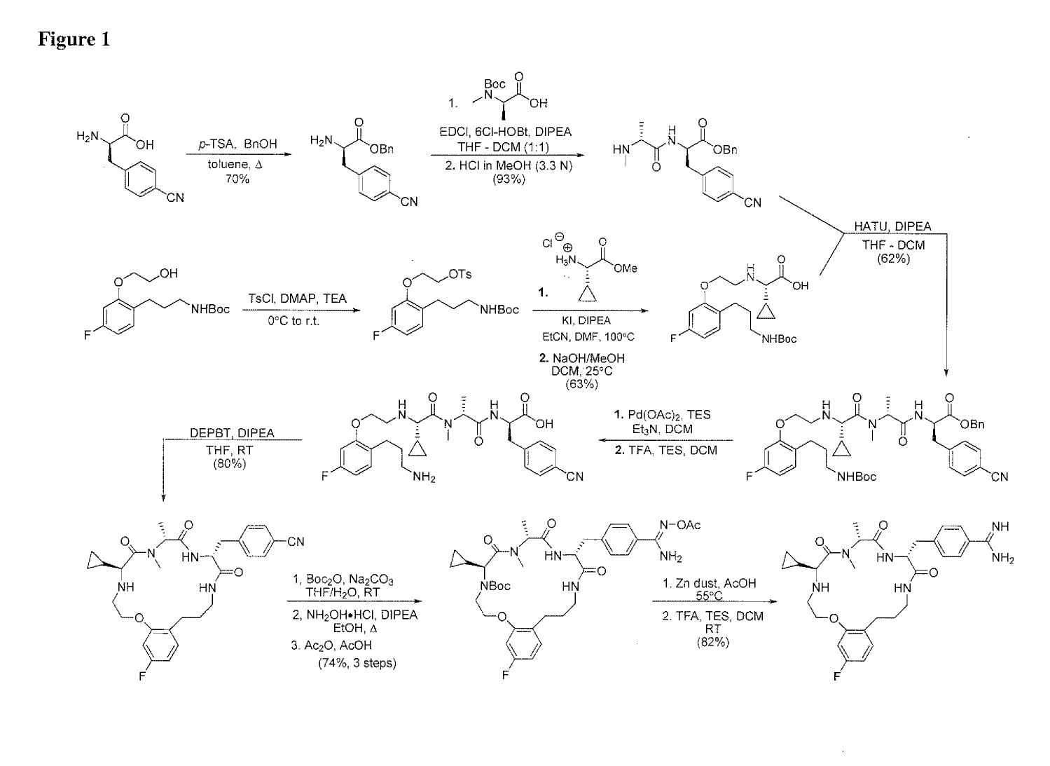

The reaction scheme for compound 451 is presented in FIG. 1.
Step 451-1. Synthesis of H-Phe(4CN)-OBn. To a toluene (75 mL) solution of H-Phe(4CN)—OH (2.85 g, 15 mmol, 1.0 eq), p-TSA (3.42 g, 18 mmol, 1.2 eq), BnOH (7.8 mL, 75 mmol, 5.0 eq) were added. The mixture was heated to reflux for 4 h with removal of H₂O with a Dean-Stark trap. The mixture was allowed to cool to RT, then was diluted with Et₂O and stirred at 0° C. (ice bath) for 45 min. The resulting white precipitate was filtered and rinsed with cold Et₂O. The white solid was dissolved in a 1M Na₂CO₃ solution, then stirred at RT for 30 min. The resulting aqueous phase was washed with EtOAc (4×). The combined organic phases were washed with brine, dried over Na₂SO₄, filtered and evaporated under reduced pressure to afford a pale orange oil (3.10 g, 70% yield).

¹H NMR: δ 1.60 (br s, 2H), 3.02 (dq, 2H), 3.77 (t, 1H), 5.13 (q, 2H), 7.21-7.52 (m, 9H)

Step 451-2. Dipeptide Formation. To a solution of H-Phe(4CN)—OBn (2.9 g, 10.27 mmol, 1.0 eq) in a THF-DCM mixture (1:1, 25 mL), Boc-NMeAla (2.15 g, 10.6 mmol, 1.03 eq), 6-Cl—HOBt (1.74 g, 10.3 mmol, 1.1 eq) were added at 0° C. (ice bath). DIPEA (8.94 mL, 51.35 mmol, 5.0 eq) and then EDCI (2.17 g, 11.3 mmol, 1.1 eq) were added and the mixture was allowed to stir at RT overnight. The volatiles were evaporated under reduced pressure and the resulting crude oil was dissolved in EtOAc. The solution was washed sequentially with 1M citrate buffer (pH=3.5, 2×), H₂O, saturated NaHCO₃ and brine, then was dried over Na₂SO₄, filtered and evaporated under reduced pressure. The combined organic layers were washed with H₂O, saturated NH₄Cl, brine, dried over Na₂SO₄, filtered and evaporated under vacuum. The crude product was purified by flash chromatography (gradient, 40% then 50% EtOAc/Hex) to provide the protected dipeptide, 4.50 g (93%).

TLC (50% EtOAc/Hex): R_{f}: =0.15, det: UV, ninhydrin,

The protected dipeptide (4.46 g, 9.6 mmol, 1.0 eq) was dissolved in a solution of 3.3 N HCl in MeOH (30 mL, 96 mmol, 10 eq). The mixture was stirred at RT for 1 h. Volatiles were then evaporated under reduced pressure and the resulting crude oil dried under vacuum (oil pump) to afford the desired compound as an amorphous solid (3.50 g, 100%).
Step 451-3. Synthesis of Boc-T69-OTs. To a DCM (36 mL) solution of Boc-T69 (4.94 g, 14.7 mmol, 1.05 eq), DMAP (342 mg g, 2.8 mmol, 0.2 eq) and Et₃N (9.8 mL, 70 mmol, 5.0 eq) were added and the mixture stirred at 0° C. (ice bath) for 15 min. A DCM solution (24 mL) of TsCl (2.67 g, 14 mmol, 1.0 eq) was then added portionwise at 0° C. The mixture was stirred at 0° C. for 45 min, then overnight at RT. A saturated solution of NH₄Cl was added, the two phases separated and the aqueous phase washed with DCM (3×). The combined organic phases were washed with 1M HCl (2×) and brine, dried over Na₂SO₄, filtered and evaporated under reduced pressure. The crude product was used without further purification for the next step (6.90 g, 100%).

¹H NMR (CDCl₃): δ 1.34 (s, 9H), 1.60 (m, 2H), 2.36 (s, 3H), 2.44 (m, 2H), 2.99 (m, 3H), 4.04 (m, 2H), 4.30 (m, 2H), 4.59 (br s, 1H), 6.35 (m, 1H), 6.50 (m, 1H), 6.94 (m, 1H), 7.26 (d, J=8.4 Hz, 2H), 7.72 (d, 1=8.4 Hz, 2H)

Step 451-4. Synthesis of Boc-T69-Cpg-OMe To a solution of Boc-T69-OTs (6.9 g, 14.7 mmol, 1 eq) in a EtCN/DMF mixture (3:1, 20 mL), H-Cpg-OMe.HCl (3.65 g, 22.1 mmol, 1.5 eq), KI (dried in oven overnight, 6.09 g, 36.7 mmol, 2.5 eq) and DIPEA (7.7 mL, 44.1 mmol, 3.0 equiv) were added at RT. The reaction mixture was stirred at 108° C. for 30 h with monitoring by LC-MS. The reaction was allowed to cool to RT, then quenched with H₂O. The mixture was diluted with EtOAc and the aqueous phase washed with EtOAc (3×). The combined organic phases were washed sequentially with 1M citrate buffer (pH=3.5), H₂O, saturated NaHCO₃ and brine, dried over Na₂SO₄, filtered and evaporated under reduced pressure. The crude product was used without further purification for the next step (5.98 g, 96%).

LC-MS: t_{R}=6.24 min (A4b), [M+H]⁺ 425

Step 451-5. Synthesis of Boc-T69-Cpg-OH. To a solution of Boc-T69-Cpg-OMe (5.98 g, 14.0 mmol, 1.0 eq) in DCM/MeOH mixture (9:1, 90 mL) was added a 2M NaOH solution in MeOH (14.1 mL, 28.2 mmol, 2.0 eq). The mixture was stirred for 48-72 h at RT. The volatiles were evaporated under reduced pressure and the residue diluted with water. The aqueous phase was washed with Et₂O, then was acidified to pH=1-2. The acid phase was washed with EtOAc (3×). The combined organic phases were washed with saturated NH₄Cl and brine, dried over Na₂SO₄, filtered and evaporated under reduced pressure. The crude solid was triturated with a Hex/DCM mixture (9:1) to afford a white solid (3.76 g, 65%).

LC-MS: t_{R}=6.12 min (A4b), [M+H]⁺ 411

Step 451-6. Fragment coupling. To a solution of Boc-T69-Cpg-OH (3.60 g, 9.2 mmol, 1.0 eq) in a DCM/THF mixture (1:1, 90 mL), H-NMeAla-Phe(4CN)-OBn.HCl (3.36 g, 9.20 mmol, 1.05 eq) was added and the mixture stirred at 0° C. (ice bath) for 15 min. DIPEA (9.23 mL, 53 mmol, 6.0 eq), and then HATU (3.50 g, 9.20 mmol, 1.05 eq) were added and the mixture for 48-72 h at RT with LC-MS monitoring. The mixture was diluted with EtOAc and washed sequentially with 1M citrate buffer (pH=3.5), H₂O, saturated NaHCO₃ and brine. The organic phase was dried over Na₂SO₄, filtered and evaporated under reduced pressure. The crude product was purified by flash chromatography (gradient 50% EtOAc/Hex, then 100% EtOAc) to give the coupled product (4.35 g, 62%).

TLC (50% EtOAc/Hex): R_{f}: =0.10, detection: UV, ninhydrin

LC-MS: t_{R}=7.95 min (A4b), [M+H]⁺ 758

Step 451-7. Deprotection. To a DCM (53 mL) solution of tripeptide-tether (4.0 g, 5.28 mmol, 1.0 eq) were added Pd(OAc)₂ (60 mg, 0.264 mmol, 0.05 eq), Et₃N (95 μL, 0.68 mmol, 0.13 eq). The mixture was degassed with Ar/vacuum cycles over 30 min. and stirred overnight at RT under argon. The volatiles were evaporated under reduced pressure and the crude dark oil filtered through a short pad of Florisil® eluted first with EtOAc, then MeOH and the combined filtrates concentrated under reduced pressure. The crude product was obtained as a pale yellow oil (3.11 g, 90%).

LC-MS: t_{R}=6.64 min (A4b), [M+H]⁺ 668

A solution of the crude oil (3.1 g, 4.57 mmol, 1.0 eq) in a DCM/TFA/TES mixture (64:33:3, 30 mL) was stirred at RT for 45 min. The volatiles were evaporated under reduced pressure. The residue was dissolved in a DCM/toluene mixture (1:1, 15 mL) and concentrated under reduced pressure. The resulting oil was used for the next step without further purification.
Step 451-8. Macrocyclization. To a THF (457 mL, c=0.01 M) solution containing the previous crude oil (3.1 g, 4.57 mmol, 1.0 eq), DIPEA (5.60 mL, 32.0 mmol, 7.0 eq) and finally DEPBT (1.50 g, 5.03 mmol, 1.1 eq) were added. The mixture was stirred at RT overnight. The volatiles were evaporated under reduced pressure and the resulting crude oil dissolved in a mixture of EtOAe/NaHCO₃ (sat) (1:1). The aqueous phase was washed with EtOAc (3×). The combined organic phases were washed with brine, dried over Na₂SO₄, filtered and evaporated under reduced pressure. The crude product was purified by flash chromatography (gradient, 0.5%/3%/96.5% AcOH/MeOH/EtOAc, 100% EtOAc, then 0.5%/3%/96.5% NH₄OH/MeOH/EtOAc to give the cyclic product (2.0 g, 80%). TLC (3% EtOAc/MeOH): R_{f}: =0.75, detection: UV, ninhydrin

LC-MS: t_{R}=5.59 min (A4b), [M+H]⁺550

Step 451-9. Boc protection. To a solution of macrocycle (2.0 g, 3.64 mmol, 1.0 eq) in a THF/H₂O mixture (1:1, 40 mL), Na₂CO₃ (1.93 g, 18.2 mmol, 5.0 eq) and Boc₂O (5.01 mL, 21.84 mmol, 6.0 eq) were added and the mixture stirred for 48-72 h at RT. The mixture was quenched with NH₄Cl (sat), then the aqueous phase washed with EtOAc (3×). The combined organic phases were washed with brine, dried over Na₂SO₄, filtered and evaporated under reduced pressure. The Boc-protected macrocycle was used as obtained for the next step.

LC-MS: t_{R}=9.14 min (A4b), [M+H]⁺ 650

Step 451-10: N-Hydroxyamidine formation. To a solution of the macrocycle (2.2 g, 3.35 mmol, 1.0 eq) in absolute EtOH (35 mL), NH₂OH.HC1 (0.750 g, 10.74 mmol, 3.2 eq), and DIPEA (2.04 mL, 11.72 mmol, 3.5 eq) were added and the resulting mixture heated to reflux overnight. The mixture was allowed to cool to RT, then the volatiles evaporated under reduced pressure. The resulting yellow clear oil was used directly for the next step.

LC-MS: t_{R}=7.20 min (A4b), [M+H]⁺ 683

Step 451-11. N-Acetoxyamidine formation. To a solution of macrocycle (2.2 g, 3.35 mmol, 1.0 eq) in AcOH (35 mL) stirred for 10 min, Ac₂O (2 mL, 16.75 mmol, 5.0 eq) was added. The resulting mixture was stirred at r.t. for 2.5 h. The volatiles were evaporated under reduced pressure. The resulting crude oil was purified by flash chromatography (10% MeOH/EtOAc) to give the desired product (1.80 g, 74% over 3 steps).

LC-MS: t_{R}=13.12 min (A4b), [M+H]⁺ 725; [M+2H-Boc]⁺ 625

Step 451-12. Amidine formation. To a solution of the macrocycle from the previous step (1.40 g, 1.93 mmol, 1.0 eq) in AcOH (35 mL) was added Zn dust (1.26 g, 19.3 mmol, 10.0 eq). The resulting mixture was stirred at 55° C. overnight. The mixture was allowed to cool to RT, then the mixture filtered through a short pad of cotton. The cotton was eluted with AcOH and, finally, EtOAc. The volatiles were evaporated under reduced pressure. The resulting yellow clear oil was able to be used directly for the next step.
Step 451-13. Boc cleavage. The macrocycle (1.40 g, 1.93 mmol, 1.0 eq) was dissolved in a DCM-TFA-TES mixture (64%-33%-3%, 20 mL) and stirred at rt for 1.5 h. The mixture was concentrated in vacuo. The crude oil was dissolved in THF, then the solvent evaporated under reduced pressure. This procedure was repeated with toluene and then EtOAc as solvents. The resulting crude oil was purified by flash chromatography (20% MeOH/DCM with 0.5% TFA, then 30% MeOH/DCM with 0.5% TFA).

TLC (30% MeOH/DCM with 0.5% TFA): Rₜ: 0.61, detection: UV, ninhydrin The macrocycle.TFA salt was dissolved in EtOAc then aqueous 1M Na₂CO₃ solution added. The aqueous phase was extracted with EtOAc (3×). The combined organic phases were washed with brine, dried over Na₂SO₄, filtered and evaporated under reduced pressure. The desired macrocycle was obtained as a white solid (0.90 g, 82%). Only one diastereoisomer was observed by ¹H NMR. If impurities were seen in the LC-MS, trituration with THF or CH₃CN could be used to improve the purity.

LC-MS: t_{R}=4.49 min (A4b), [M+H]⁺ 567

The deprotection could also be achieved by treatment with 4M HCl in dioxane. The crude macrocycle in that case was purified by flash chromatography (30% MeOH/DCM with 0.5% TFA). On a 120 mg scale, 66% yield over the two steps was obtained.
Step 451-14. Formation of HCl salt: The compound was dissolved in acetonitrile, then 0.1 N HCl (4 eq) was added, and the solution lyophilized overnight. The resulting solid was triturated with THF.

LC-MS: t_{R}=6.14 min (84), [M+H]⁺ 567

The amidino group alternatively could be synthesized without using Boc-protection on the secondary amine of the macrocycle as shown:

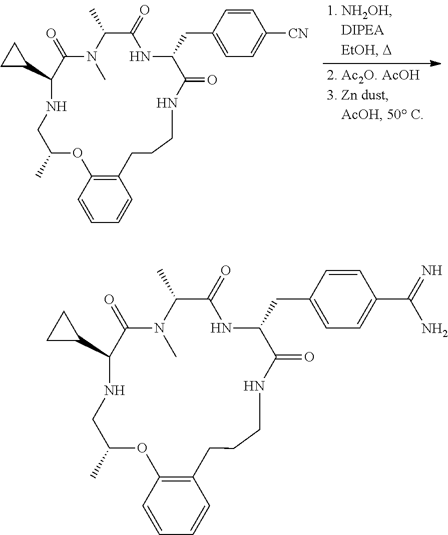

An additional alternative approach is to synthesize the amidino-containing macrocycle directly from the corresponding cyano precursor using the following conditions. (Garigipati, R. S. Tetrahedron Lett. 1990, 31, 1969.)

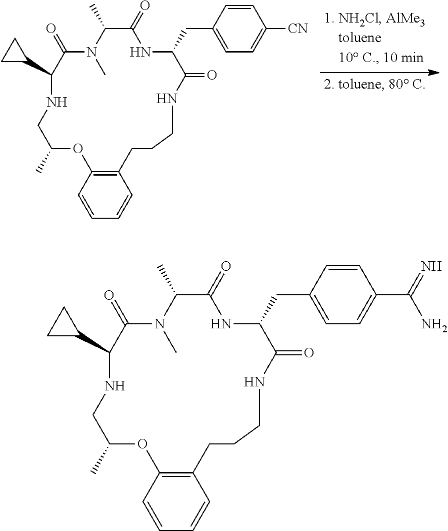

### B. Standard Procedure for the Simultaneous Synthesis of Multiple Representative Compounds of the Invention

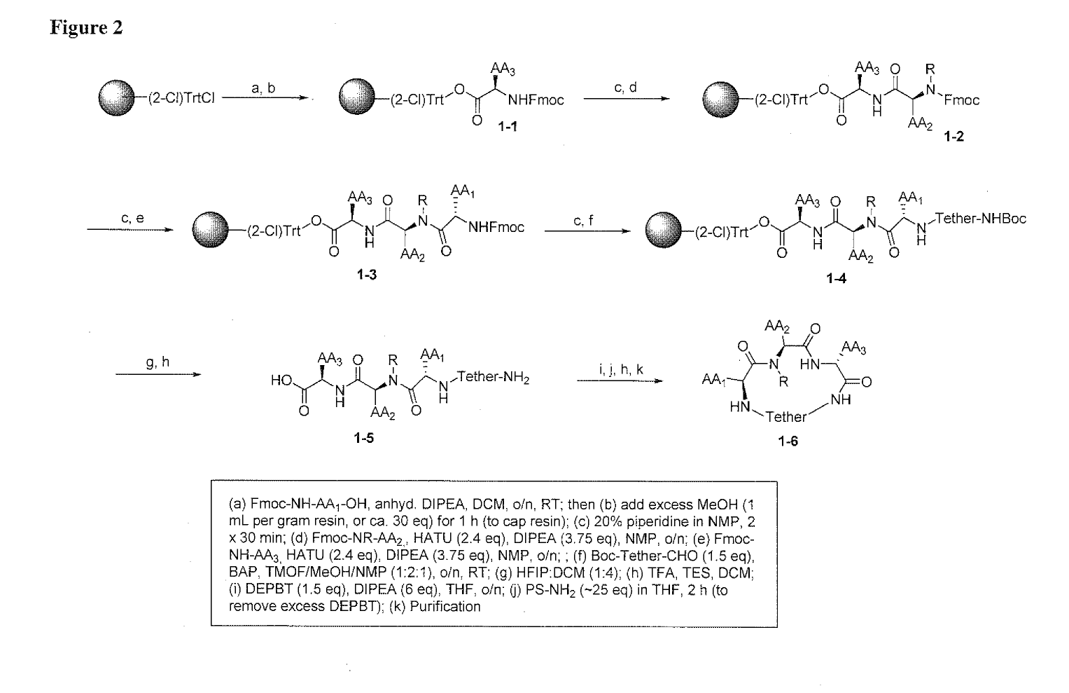

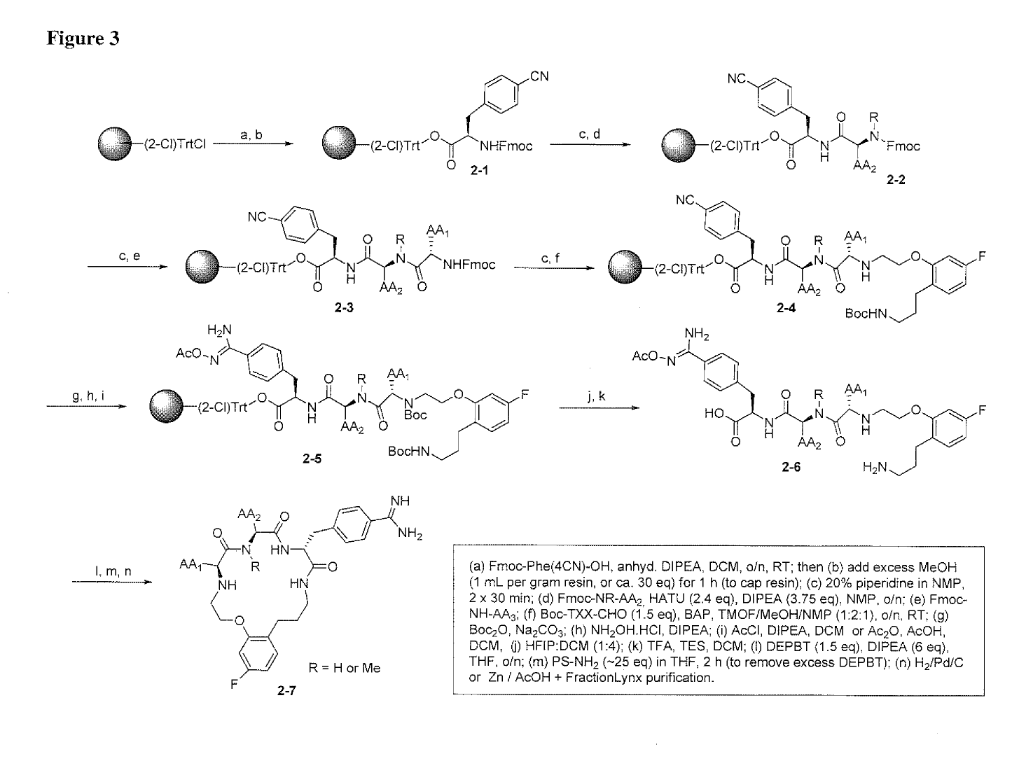

The standard reaction schemes are presented in FIGS. 2 and 3.

The following procedure uses a particular technique, involving radiofrequency tagging, that enables ease of tracking of multiple reactions conducted simultaneously for multiple individual compounds. However, this was not required and the solid phase syntheses can also be conducted similarly in individual reaction vessels.

Step B-1. AA₃ loading. 2-Chlorotrityl chloride resin was loaded into MiniKans (or other appropriate separatable reaction vessel) and washed with DCM for 15 min. DCM was removed and a solution of DIPEA (4 eq) and Fmoc-NH-AA₃ (2 eq) added (using separate vessels with MiniKans for each separate AA₃). The reaction mixtures were agitated on an orbital shaker overnight at RT. The MiniKans were washed twice with the following cycle DCM, iPrOH, DCM, then dried under a flow of N₂.

One MiniKan (for QC), or part of the resin was removed from one MiniKan, was reacted in an HFIP:DCM (1:4, 5 mL) mixture and agitated for at least 30 min at RT on an orbital shaker. The resin was washed with DCM and the volatiles evaporated under reduced pressure. The crude oil so obtained was then submitted to quantitative QC analysis for estimation of loading efficiency.

Step B-2. Fmoc-deprotection. The MiniKans were treated with a 20% piperidine solution in NMP (3.5 mL/MiniKan), then agitated on an orbital shaker for 30 min. This treatment was then repeated. The MiniKans were washed with the following sequence: NMP (2×), WA, DCM, IPA, DCM (3×), then dried under a flow of N₂.

Step B-3. AA₂ coupling. Fmoc-NR-AA₂-OH (2.5 eq) was dissolved in NMP, then DIPEA (5 eq) followed by HATU (2.5 eq) added. The mixture was stirred at RT for 10 min, then transferred to the appropriate set of MiniKans (segregated by AA₂ into separate vessels) and agitated on an orbital shaker at RT overnight. The MiniKans were washed with the following sequence: NMP (2×), IPA, DCM, IPA, DCM (3×), then dried under a flow of N₂.

Step B-4. Fmoc-deprotection. The MiniKans were treated with a 20% piperidine solution in NMP (3.5 mL/MiniKan), then agitated on an orbital shaker for 30 min. This treatment was then repeated. The MiniKans were washed with the following sequence: NMP (2×), IPA, DCM, IPA, DCM (3×), then dried under a flow of N₂.

Step B-5. AA₁ coupling. Fmoc-NH-AA₁-OH (2.5 eq) was dissolved in NMP, then DIPEA (5 eq) followed by HATU (2.5 eq) added. The mixture was stirred at RT for 10 min, then transferred to the appropriate set of MiniKans (segregated by AA, into separate vessels) and agitated on an orbital shaker at RT overnight. The MiniKans were washed with the following sequence: NMP (2×), IPA, DCM, IPA, DCM (3×), then dried under a flow of N₂.

Step B-6A. Tether oxidation. To a DMSO solution of tether was added IBX (1.5 eq) added. The heterogeneous mixture was stirred at RT for 5 min, then H₂O added and the stirring maintained overnight at RT. The mixture was quenched by water (a white precipitate was formed), and the solution stirred for 20 min at RT. The solid was removed by filtration, washed with EtOAc and the resulting solution was washed with aq. NaHCO₃ and brine, dried over MgSO₄, then concentrated under reduced pressure. The crude aldehyde was dried under vacuum, the structure confirmed by ¹H NMR, then used as such for the next step.

Step B-6B. Reductive amination. The MiniKans were treated with a 20% piperidine solution in NMP (3.5 mL/MiniKan), then agitated on an orbital shaker for 30 min. This treatment was then repeated. The MiniKans were washed with the following sequence: NMP (2×), IPA, DCM, IPA, DCM (3×), then dried under a flow of N₂. The crude tether aldehyde from Step 6A was dissolved in a mixture of TMOF-MeOH (1:3). The resulting solution was transferred into the vessel containing the appropriate MiniKans (separated by Tether) and agitated at RT for 10 min on orbital shaker. The BAP reagent (2 eq) was added and the agitation maintained overnight at RT. INote that gas is evolved and the container must be sealed tightly (or vented) to avoid loss of solvent.] The MiniKans were washed with the following sequence: DCM (2×), THF-DCM/MeOH (3:1), THF/MeOH (3:1), DCM (3×), then dried under a flow of N₂.

Step B-7. Formation of the N-hydroxyamidine. First, a 1 M solution of NH₂OH in NMP was prepared as follows 3.51 g of NH₂OH.HC1 was dissolved in DIPEA (9.2 mL), then the volume adjusted to 50 mL with NMP. The heterogenous mixture was stirred at RT until complete dissolution of the residual salts. The MiniKans were treated with NMP (4 mL/MiniKan), the solution degassed with a N₂/vacuum cycle (30 min), then the 1 M NMP solution of NH₂OH was added (2 mL/MinKan) and the mixture stirred at 50° C. (oil bath) for 24 h. The solution was allowed to cool to RT. The MiniKans were washed with the following sequence: NMP (2×), IPA, NMP, IPA, THF.DCM/MeOH (3:1), DCM (3×), then dried under a flow of N₂.

Step B-8. Cleavage from resin. The resin was removed from the individual MiniKans and introduced to separate 20 mL reactor vessels. A solution of HFIP/DCM (1:4) was added and the resulting red solution agitated on an orbital shaker for 1 h. The resin was removed by filtration, washed with DCM, and the volatiles evaporated in vacuo (using a SpeedVac centrifugal evaporator for multiple samples).

Step B-9. N-Acetoxyamidine formation. Note that the stoichiometry presented in Steps B-9 to B-11 is based on 250 μmol of tripeptide (theoretical yield) and can be adjusted proportionally for other quantities. The individual oils from Step 8 were dissolved in AcOH (2.5 mL) and the solution stirred at RT for 10 min, then Ac₂O added (0.15 mL, 1.25 mmol, 5 eq) and the stirring continued for 45 min. The volatiles were evaporated in vacuo (using a SpeedVac centrifugal evaporator for multiple samples).

Step B-10. Tether deprotection and macrocyclization. The individual residues from Step B-9 were dissolved in a TES-TFA-DCM mixture (3:33:64, 5 mL) and the solution stirred at RT for 45 min. The volatiles were evaporated in vacuo (using a SpeedVac centrifugal evaporator for multiple samples), then the residue dissolved in toluene and again concentrated in vacuo (on SpeedVac).

For a Ddz-protected tether, a mixture of TFA-TES-DCM (2:3:95) was used for the deprotection step. It is important not to exceed 1 h during Ddz deprotection because of the potential for Boc-side chain deprotection to occur.

The individual oils were dissolved in THF (25 mL), then DIPEA (300 μL, 1.75 mmol, 7 eq) followed by DEPBT (0.150 g, 0.50 mmol, 2 eq) added. The yellow solution was agitated on an orbital shaker overnight at RT. Si-Trisamine resin was introduced (3.5 g per reaction) and the resulting mixture agitated for 2 h on an orbital shaker at RT. The resin was removed by filtration, washed with THF and the volatiles evaporated in vacuo (using a SpeedVac centrifugal evaporator for multiple samples).

Step B-11. Amidine formation. The oils from Step 10 were dissolved in AcOH (3 mL), then Zn dust (0.163 g, 2.5 mmol, 10 eq) added and the solution agitated overnight at RT on an orbital shaker. The excess of Zn dust was removed using a short pad of cotton, then eluted with AcOH. The volatiles were evaporated in vacuo (using a SpeedVac centrifugal evaporator for multiple samples). then the residues subjected to Fraction Lynx purification to obtain the desired products.

For the cases where the desired macrocycle did not bear an amidino group, Steps B-9 and B-11 were omitted. For other specific sequences, Boc side chain deprotection at the AA₃ position was performed under standard conditions using the TFA-TES-DCM system. Additionally, Trt side chain deprotection on AA₁ position was performed under standard conditions using TFA-TES (95:5).

The foregoing is illustrative of the present invention, and is not to be construed as limiting thereof. The invention is defined by the following claims, with equivalents of the claims to be included therein.

1. A compound of the formula (I): 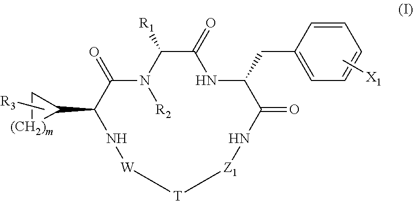
or a pharmaceutically acceptable salt thereof, wherein:
R₁ is selected from the group consisting of —H, —CH₃, —CH₂CH₃, —(CH₂)₂CH₃ and —CH(CH₃)₂;
R₂ is selected from the group consisting of —H, —CH₃ and —CH₂CH₃;
R₃ is optionally present and is selected from the group consisting of C₁-C₄ alkyl, hydroxyl and alkoxy;
m is 1, 2, 3, 4 or 5;
X₁ is selected from the group consisting of amidino, ureido and guanidino;
W is selected from the group consisting of CR₄ₐR_{4b}, wherein R₄ₐ and R_{4b} are independently selected from the group consisting of hydrogen, C₁-C₄ alkyl and trifluoromethyl;
Z₁ is selected from the group consisting of CR₅ₐR_{5b}, wherein R₅ₐ and R_{5b} are independently selected from the group consisting of hydrogen, C₁-C₄ alkyl and trifluoromethyl; and
T is selected from the group consisting of: 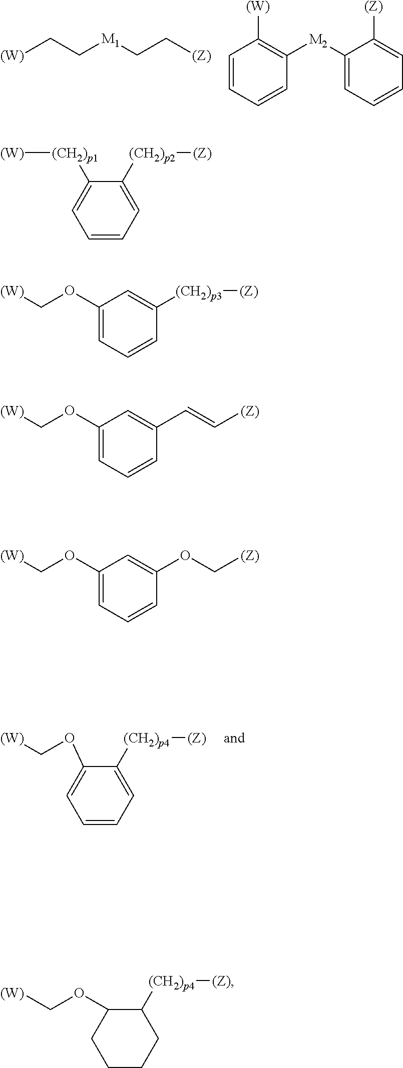
wherein M₁ is selected from the group consisting of 0 and (CH₂)_{q}, wherein q is 1, 2, 3, 4 or 5; M₂ is selected from the group consisting of O, S, NR₆ and CR₇ₐR_{7b}, wherein R₆ is selected from the group consisting of hydrogen, alkyl, formyl, acyl, carboxyalkyl, carboxyaryl, amido, sulfonyl and sulfonamido; R₇ₐ and R_{7b} are independently selected from the group consisting of hydrogen, hydroxyl, alkoxy, C₁-C₄ alkyl and trifluoromethyl; p1 and p2 are independently 0, 1, 2 or 3; and p3, p4 and p5 are independently 0, 1 or 2.
(W) indicates the site of bonding to the attached carbon atom of W.
(Z) indicates the site of bonding to the attached carbon atom of Z₁.

2. The compound of claim 1 having the structure

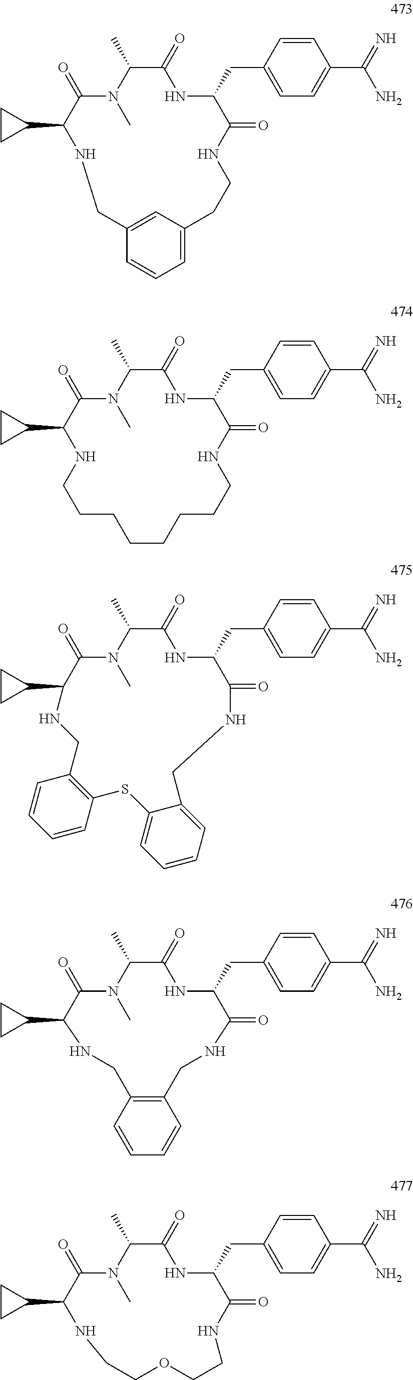

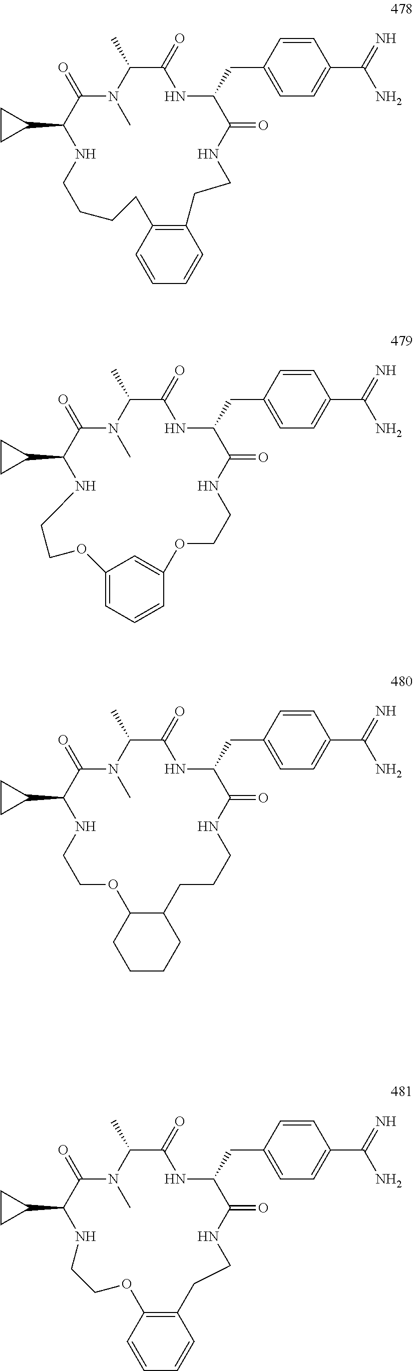

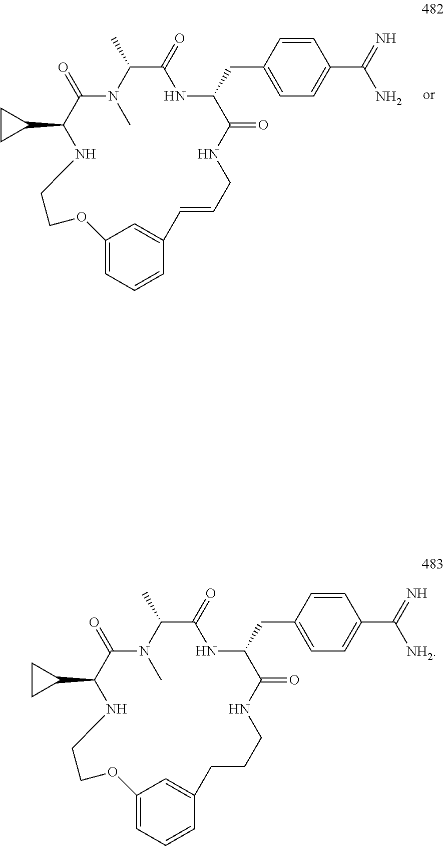

3. A pharmaceutical composition comprising:
(a) a compound of formula (I) of claim 1; and
(b) a pharmaceutically acceptable carrier, excipient or diluent.

4. A pharmaceutical composition comprising:
(a) a compound of claim 2; and
(b) a pharmaceutically acceptable carrier, excipient or diluent.

5. A compound of the formula (II): 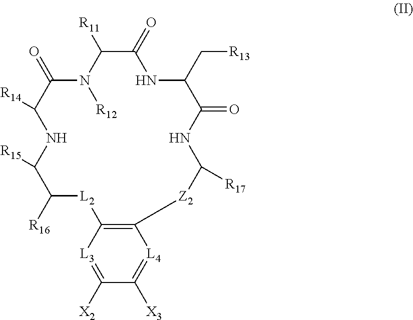
or a pharmaceutically acceptable salt thereof, wherein:
R₁₁ is selected from the group consisting of —H, —CH₃, —CH₂CH₃, —(CH₂)₂CH₃ and —CH(CH₃)₂;
R₁₂ is selected from the group consisting of —H, —CH₃ and —CH₂CH₃;
R₁₃ is selected from the group consisting of —(CH₂)ᵣ₁NR₁₈ₐR_{18b}, —(CH₂)ᵣ₂CONR₁₉ₐR_{19b}, 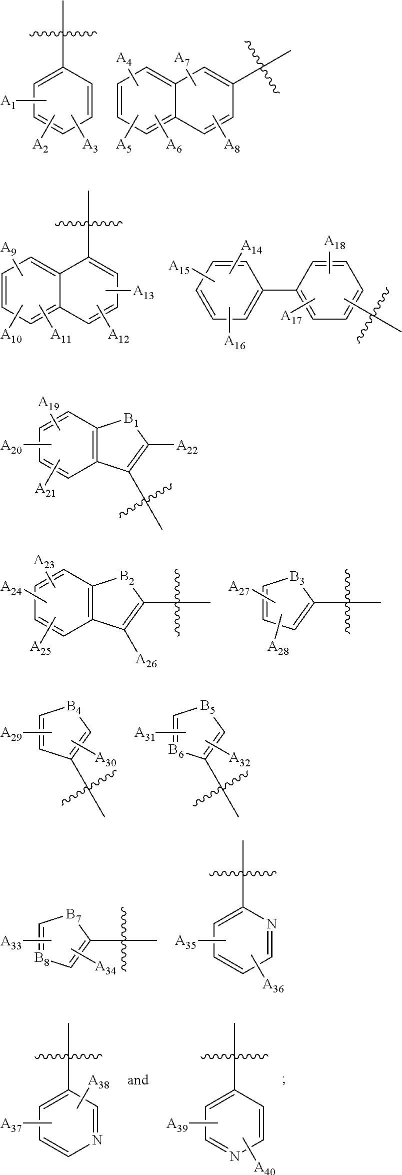
wherein r1 is 1, 2, 3, 4 or 5; r2 is 1, 2 or 3; R₁₈ₐ, R₁₉ₐ and R_{19b} are independently selected from the group consisting of hydrogen and C₁-C₄ alkyl; R_{18b} is selected from the group consisting of hydrogen, C₁-C₄ alkyl, acyl, amido, amidino, sulfonamido; A₁, A₄, A₇, A₉, A₁₂, A₁₄, A₁₇, A₁₉, A₂₃, A₃₅, A₃₇ and A₃₉ are each optionally present and are independently selected from the group consisting of halogen, trifluoromethyl, amidino, ureido, guanidino, hydroxyl, alkoxy and C₁-C₄ alkyl; A₂, A₃, A₅, A₆, A₈, A₁₀, A₁₁, A₁₃, A₁₅, A₁₆, A₁₈, A₂₀, A₂₁, A₂₄, A₂₅, A₃₆, A₃₈ and A₄₀ are each optionally present and are independently selected from the group consisting of halogen, trifluoromethyl, hydroxyl, alkoxy and C₁-C₄ alkyl; A₂₂, A₂₆, A₂₇, A₂₉, A₃₁ and A₃₃ are each optionally present and are independently selected from the group consisting of trifluoromethyl, amidino, ureido, guanidino and C₁-C₄ alkyl; A₂₈, A₃₀, A₃₂ and A₃₄ are each optionally present and are independently selected from the group consisting of trifluoromethyl and C₁-C₄ alkyl; and B₁, B₂, B₃, B₄, B₅ and B₇ are independently NR₂₀, S or O, wherein R₂₀ is selected from the group consisting of hydrogen, alkyl, formyl, acyl, carboxyalkyl, carboxyaryl, amido, sulfonyl and sulfonamido; and B₆ and B₈ are independently N or CH;
R₁₄ is selected from the group consisting of C₁-C₄ alkyl, optionally substituted with amino, hydroxyl, alkoxy, carboxy, ureido, amidino, or guanidine, and C₃-C₇ cycloalkyl, optionally substituted with alkyl, hydroxyl or alkoxy;
R₁₅ and R₁₆ are independently selected from the group consisting of hydrogen, C₁-C₄ alkyl, hydroxyl and alkoxy;
R₁₇ is selected from the group consisting of hydrogen and C₁-C₄ alkyl;
n is 1, 2, 3, 4 or 5;
Z₂ is selected from the group consisting of CHR₂₁ₐCHR₂₂ₐ, CR_{21b}═CR_{22b} and C≡C, wherein R₂₁ₐ and R₂₂ₐ are independently selected from the group consisting of hydrogen, C₁-C₄ alkyl, hydroxyl and alkoxy; or R₂₁ₐ and R₂₂ₐ together with the carbons to which they are bonded form a three-membered ring; and R_{21b} and R_{22b} are independently selected from the group consisting of hydrogen and C₁-C₄ alkyl;
X₂ is selected from the group consisting of hydrogen, halogen, amidino, ureido and guanidino;
X₃ is selected from the group consisting of hydrogen, hydroxyl, alkoxy, amino, halogen, trifluoromethyl and C₁-C₄ alkyl;
L₂ is selected from the group consisting of O and CR₂₃ₐR_{23b}, wherein R₂₃ₐ is selected from the group consisting of hydrogen, C₁-C₄ alkyl, hydroxyl and alkoxy; and R_{23b} is selected from the group consisting of hydrogen and C₁-C₄ alkyl;
L₃ is selected from the group consisting of CX₄ and N, wherein X₄ is selected from the group consisting of hydrogen, halogen, hydroxyl, alkoxy, amino, halogen, trifluoromethyl, amidino, ureido and guanidino; and
L₄ is selected from the group consisting of CX₅ and N, wherein X₅ is selected from the group consisting of hydrogen, halogen, trifluoromethyl, hydroxyl, alkoxy, amino, amidino, ureido and guanidino.

6. The compound of claim 5 having the structure

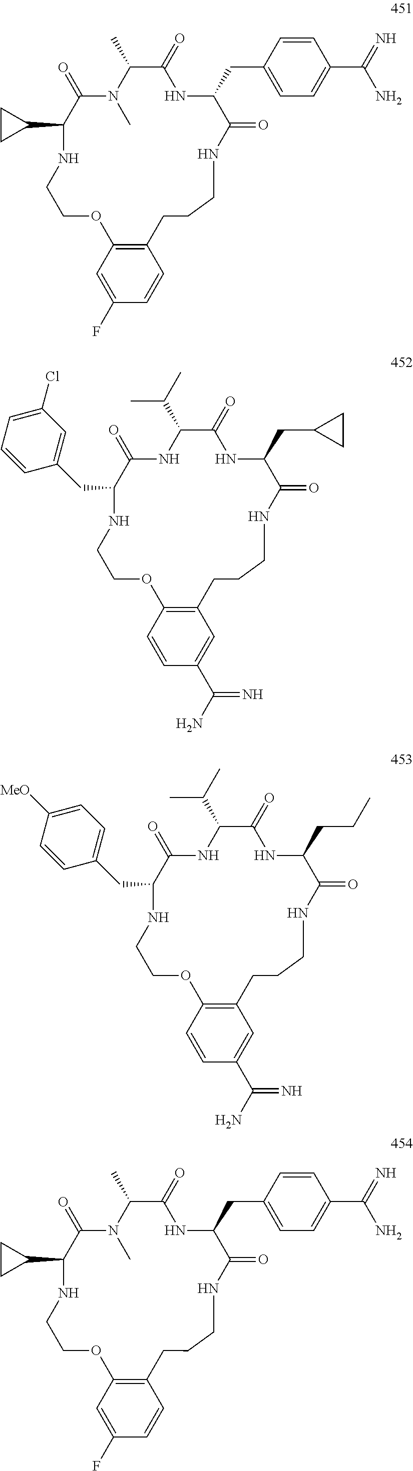

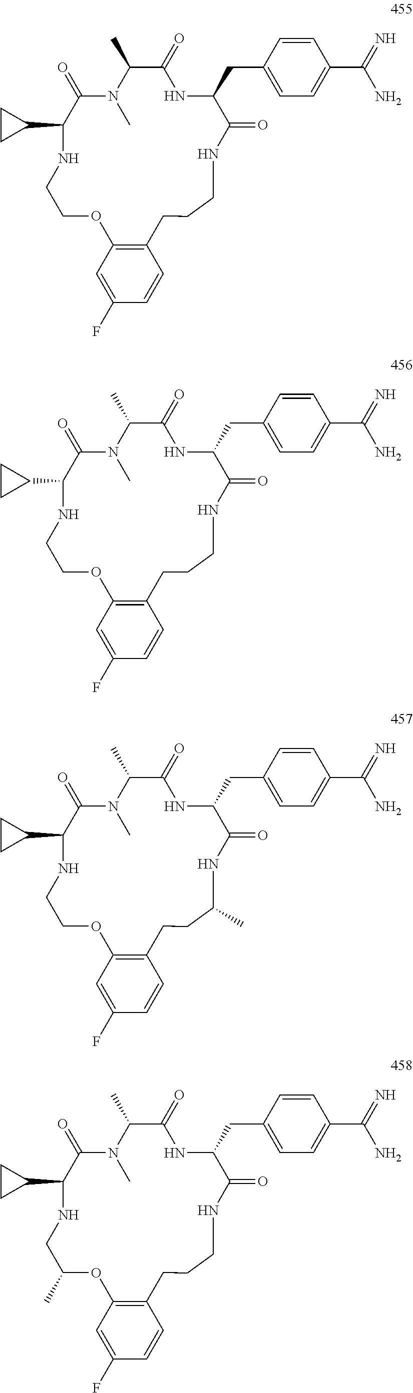

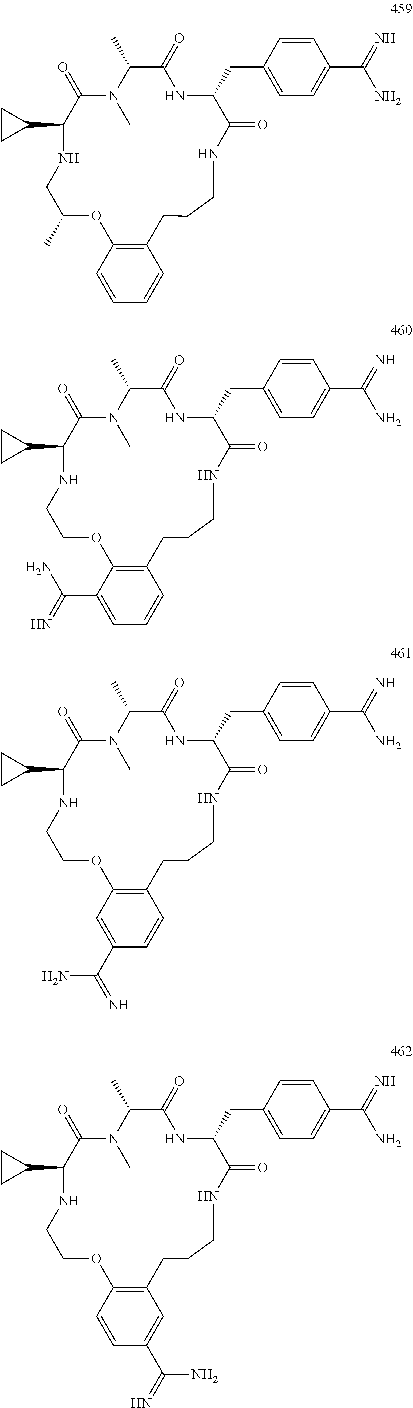

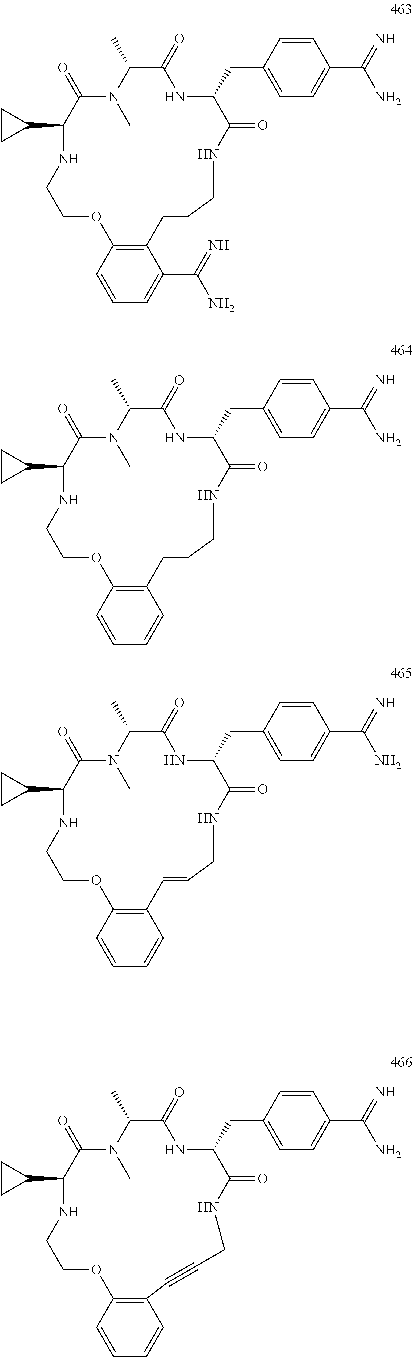

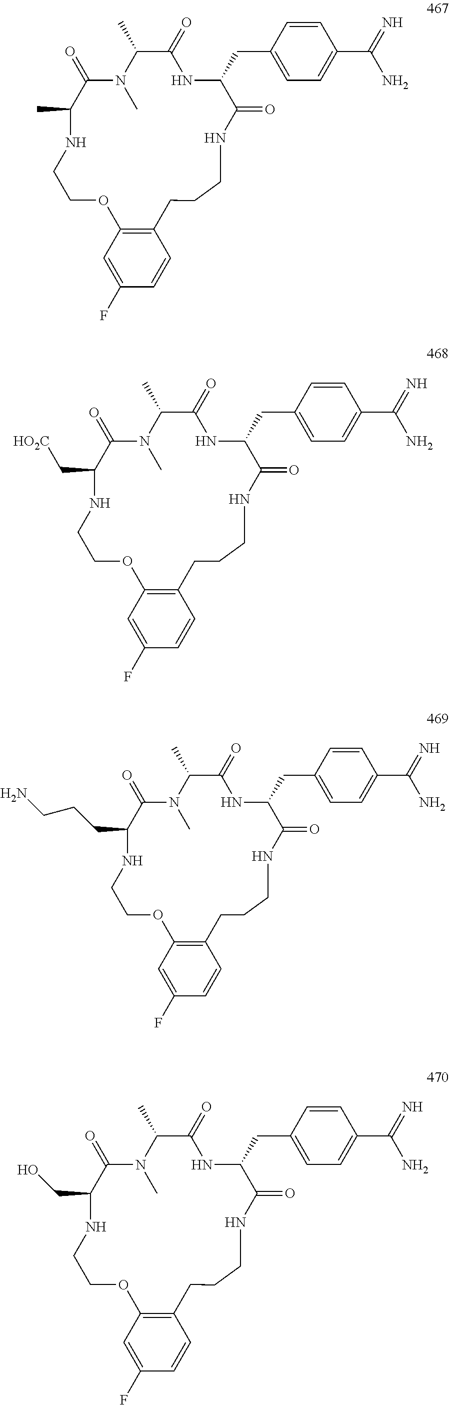

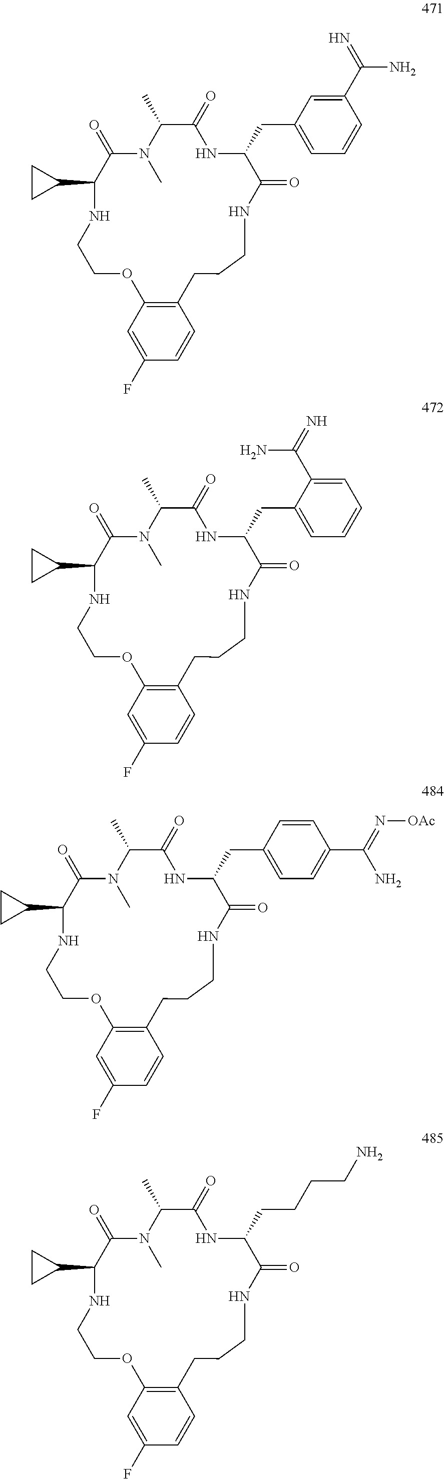

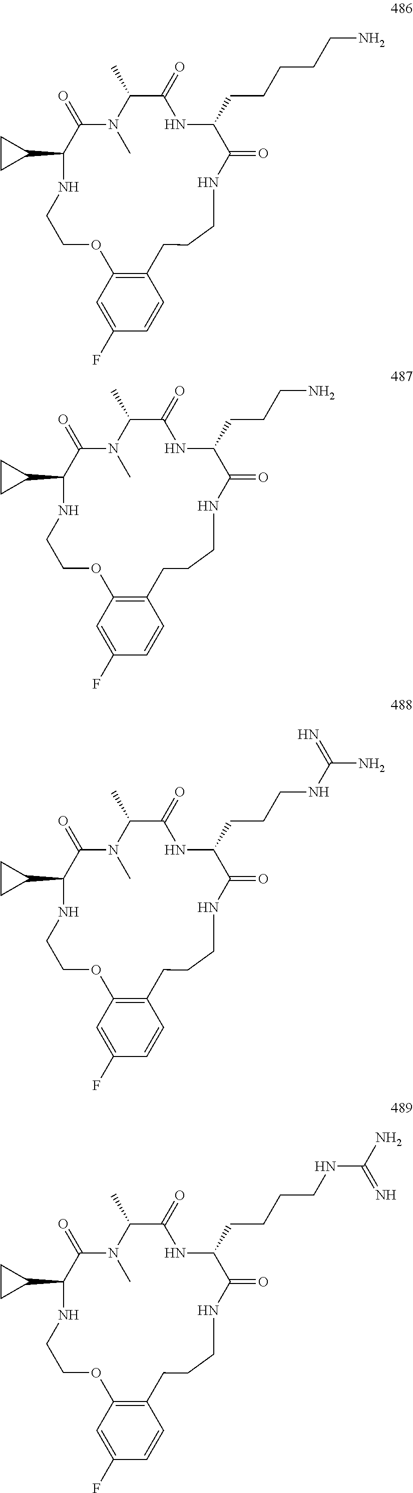

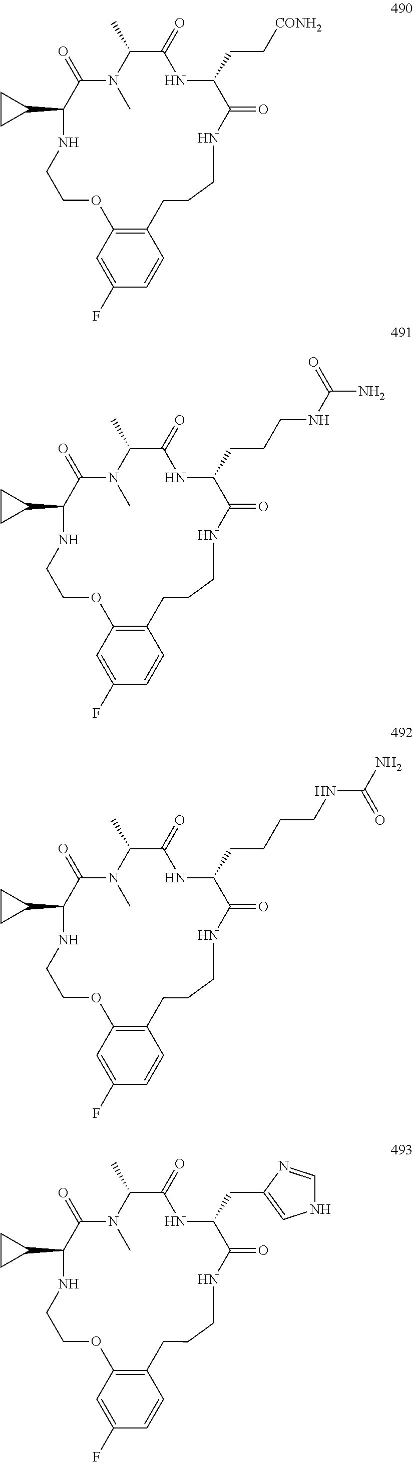

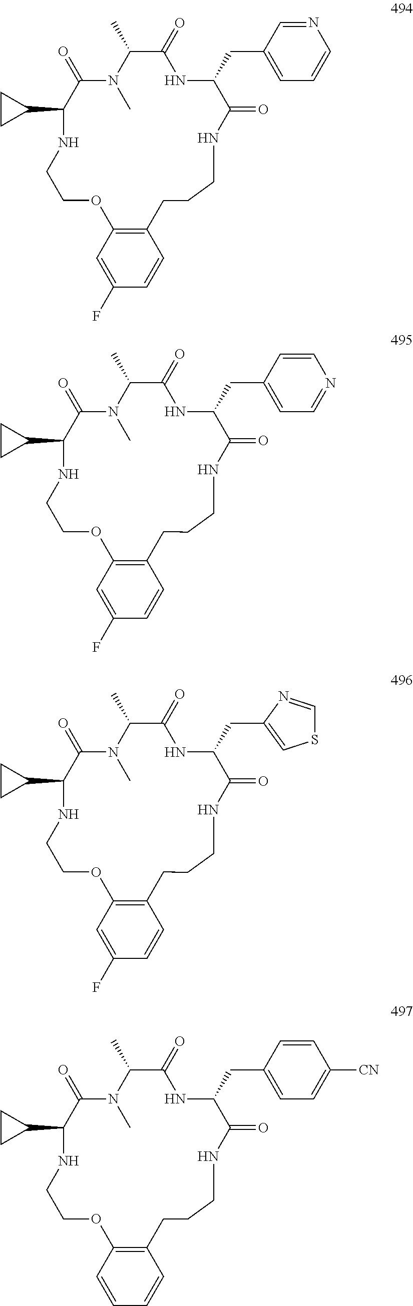

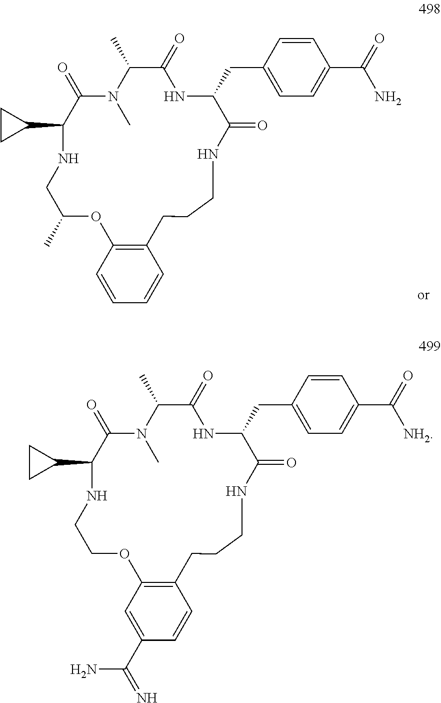

7. A pharmaceutical composition comprising:
(a) a compound of formula (II) of claim 5; and
(b) a pharmaceutically acceptable carrier, excipient or diluent.

8. A pharmaceutical composition comprising:
(a) a compound of claim 6; and
(b) a pharmaceutically acceptable carrier, excipient or diluent.
